(19) United States
(12) Patent Application Publication
(10) Pub. No.: US 2024/0036056 A1
(43) Pub. Date: Feb. 1, 2024

(54) MASS SPECTROMETRY-BASED STRATEGY FOR DETERMINING PRODUCT-RELATED VARIANTS OF A BIOLOGIC

(71) Applicant: Regeneron Pharmaceuticals, Inc., Tarrytown, NY (US)

(72) Inventors: Zhengqi Zhang, Goldens Bridge, NY (US); Yuetian Yan, Chappaqua, NY (US); Shunhai Wang, Scarsdale, NY (US); Jethro Prinston, Tarrytown, NY (US); Jennifer Nguyen, Pleasantville, NY (US)

(21) Appl. No.: 18/374,492

(22) Filed: Sep. 28, 2023

Related U.S. Application Data
(63) Continuation-in-part of application No. 18/229,354, filed on Aug. 2, 2023.
Application No. 18/229,354 is a continuation-in-part of application No. 17/885,805, filed on Aug. 11, 2022.
Application No. 17/885,805 is a continuation-in-part of application No. 17/863,303, filed on Jul. 12, 2022.
(60) Provisional application No. 63/221,436, filed on Jul. 13, 2021.

(51) Int. Cl. G01N 33/68; G01N 30/72; G01N 30/74

(52) U.S. Cl. CPC G01N 33/6848; G01N 30/7233; G01N 30/74; G01N 2030/027

(57) ABSTRACT

The present invention relates to the field of protein characterization, and in particular to methods for identifying critical quality attributes of therapeutic proteins by implementing a workflow including using a competitive binding assay with insufficient capture molecule followed by LC-MS.

### CROSS-REFERENCE TO RELATED APPLICATIONS

This application is a continuation-in-part of U.S. patent application Ser. No. 18/229,354, filed on Aug. 2, 2023, which is a continuation-in-part of U.S. patent application Ser. No. 17/885,805, filed on Aug. 11, 2022, which is a continuation-in-part of U.S. patent application Ser. No. 17/863,303, filed on Jul. 12, 2022, which claims priority to and the benefit of U.S. Provisional Patent Application No. 63/221,436, filed Jul. 13, 2021, which is herein incorporated by reference.

### FIELD

The invention generally pertains to methods for determining product-related variants critical for maintaining the structure and function of a biologic using a competitive binding-mass spectrometry workflow.

### BACKGROUND

Biologics have emerged as important drugs for the treatment of cancer, autoimmune disease, infection and cardiometabolic disorders, and they represent one of the fastest growing product segments of the pharmaceutical industry. Biologics must meet very high standards of purity. Thus, it can be important to monitor impurities at different stages of drug development, production, storage and handling. It is often difficult to fully evaluate the impact of the large number of quality attributes that may be related to safety and efficacy. The effects of manufacturing process parameters and material attributes on product quality variations are also difficult to fully characterize.

For robust manufacturing operations, it is important that an integrated control strategy is developed and improved over time based on systematic process characterization along with implementation of appropriate risk assessment and mitigation throughout the product lifecycle. Thus, there is a need for a quality by design standard. The U.N.'s World Health Organization recommends quality by design as a standard because it is harder (and practically impossible) to implement effective quality controls solely by testing a product after the fact. Critical quality attributes (CQAs) serve as the benchmarks that most quality by design implementations revolve around. A CQA is a physical, chemical, biological, or microbiological property or characteristic that should be within an appropriate limit, range, or distribution to ensure the desired product quality. CQAs are generally associated with the drug substance, excipients, intermediates (in-process materials), and drug product. For biologics, CQAs can be product- or process-related impurities. Product-related impurities can include size variants (aggregates or fragments), variants with post-translational modifications, or charge variants. Process-related impurities are an inherent part of the process, such as the host cells' DNA or host cell proteins (HCPs), leachables (such as protein A), and viruses. The presence of these impurities in the final drug product can affect product purity, product efficacy and stability.

Identifying CQAs for biologics can, therefore, be a complicated process. Currently, liquid chromatography-tandem mass spectrometry (LC-MS/MS), electrospray ionization-mass spectrometry (ESI-MS), fractionation, or variant identification can be used for physicochemical characterization of intact or digested biologics. Activity characterization can be conducted using ELISA-based bioassays, cell-based bioassays, or surface plasmon resonance (SPR) or biolayer interferometry (BLI) for binding activities. For these methods, product-related CQAs need to be enriched or isolated first and then evaluated individually or based upon experience or prior knowledge. Such an approach to a workflow can result in low throughput.

Thus, there is a long felt need in the art for an efficient method for determining such quality control attributes.

### SUMMARY

Methods have been developed for simple and high-throughput identification of potential critical quality attributes (CQAs) of a protein of interest, for example a therapeutic monoclonal antibody. A CQA may comprise, for example, a post-translational modification (PTM). This disclosure describes a competitive binding-LC-MS method, wherein a protein of interest is contacted to an insufficient amount of capture molecule, producing a flow-through enriched for variants of the protein of interest that have impaired binding. The flow-through can be subjected to separation, for example using ion exchange chromatography, and quantification, for example using UV detection and/or mass spectrometry. An abundance of potential CQAs can be compared between the flow-through and a control sample to identify attributes that are significantly enriched in the unbound pool of protein, which can therefore be identified as having an effect on binding of the protein of interest to the capture molecule.

This disclosure provides a method for characterizing at least one product-related variant, said method comprising obtaining a sample including a protein of interest and at least one product-related variant of said protein of interest; contacting said sample to a competitive binding condition including an insufficient target immobilized on beads; washing said beads to collect a flow-through; subjecting said flow-through to liquid chromatography-mass spectrometry analysis to separate said protein of interest and said at least one product-related variant; and comparing the abundance of said at least one product-related variant to an abundance of said at least one product-related variant obtained from a liquid chromatography-mass spectrometry analysis of a control sample prior to contacting said sample to said competitive binding condition to characterize said at least one product-related variant.

In one aspect of this embodiment, the target is an antigen against which the protein of interest is directed.

In one aspect of this embodiment, the binding condition provides an insufficient target immobilized on beads. In the same or another aspect of this embodiment, the at least one product-related variant has compromised binding with said insufficient target.

In one aspect of this embodiment, the liquid chromatography is cation-exchange chromatography. In a specific aspect of this embodiment, the liquid chromatography is a strong cation-exchange chromatography.

In one aspect of this embodiment, the mass spectrometer is an electrospray ionization mass spectrometer. In a specific aspect of this embodiment, the mass spectrometer is a nano-electrospray ionization mass spectrometer.

In one aspect of this embodiment, said beads are magnetic. In another aspect of this embodiment, said beads are non-magnetic. In a further aspect, said beads are agarose beads. In yet another aspect, said beads are capable of being coated with a peptide or a protein.

In one aspect of this embodiment, wherein said flow-through is enriched for said at least one product-related variant.

In the same or another aspect of this embodiment, said flow-through is collected by performing centrifugation.

In one aspect of this embodiment, said target is biotinylated before immobilizing on said beads. In the same or other aspects of this embodiment, said beads are coated with streptavidin resin. In a specific aspect of this embodiment, said beads are non-magnetic. In another specific aspect, said beads are magnetic.

In one aspect of this embodiment, said insufficient target is such that the amount of said target allows binding of about 30% to about 80% of the protein of interest. In another aspect, said insufficient target is such that the amount of said target allows binding of about 25% to about 90% of the protein of interest.

In another aspect of this embodiment, said sample is incubated for about an hour prior to washing. In the same or other aspects of this embodiment, said sample is incubated at room temperature prior to washing.

In one aspect of this embodiment, the method is capable of identifying more than one product-related variant. In a specific aspect, said product-related variant comprises a size-variant. In a specific aspect, said size-variant is a fragmentation variant of said protein of interest. In a specific aspect, said size-variant is an aggregation variant of said protein of interest.

In one aspect of the embodiment, said product-related variant comprises a charge-variant of said protein of interest. In a specific aspect, said product-related variant comprises a post translationally modified-variant of said protein of interest.

In one aspect of this embodiment, said product-related variant is classified as a critical quality attribute if said abundance of said at least product-related variant is significantly more than said abundance of said at least product-related variant in the sample prior to contacting said sample to said competitive binding condition.

This disclosure additionally provides a method for determining an effect of at least one post-translational modification (PTM) on binding of a protein of interest to a capture molecule, comprising: (a) contacting a sample including a protein of interest to a capture molecule, wherein said protein of interest features at least one PTM, said capture molecule is immobilized to a solid surface, and an amount of said capture molecule is insufficient to bind all of said protein of interest; (b) collecting unbound protein of interest to produce a flow-through; (c) contacting said flow-through to a separation column to separate at least one variant of said protein of interest featuring at least one PTM; (d) quantitating an amount of said at least one variant; (e) comparing said amount to an amount quantitated in a control sample; and (f) using said comparison to determine an effect of said at least one PTM on binding of said protein of interest to said capture molecule.

In one aspect, the protein of interest is selected from a group consisting of an antibody, an antigen-binding protein, a fusion protein, a receptor, a receptor ligand, and a therapeutic protein. In another aspect, the protein of interest is an antibody. In a specific aspect, the protein of interest is a monospecific antibody, a bispecific antibody or a trispecific antibody.

In one aspect, the capture molecule is selected from a group consisting of an antibody, an antigen, a receptor, a receptor ligand, and a therapeutic target.

In one aspect, the surface is selected from a group consisting of a microplate, resin, and beads.

In one aspect, the beads are agarose beads or magnetic beads.

In one aspect, the immobilization comprises contacting a biotinylated capture molecule to an avidin- or streptavidin-bound solid surface.

In one aspect, the collecting comprises centrifuging the solid surface.

In one aspect, the separation column is selected from a group consisting of a reverse phase chromatography column, normal phase chromatography column, hydrophobic interaction chromatography column, hydrophilic interaction chromatography column, ion exchange chromatography column, anion exchange chromatography column, cation exchange chromatography column, strong cation exchange chromatography column, and a weak cation exchange chromatography column. In another aspect, the separation column is a strong cation exchange chromatography column. In a further aspect, the separation column is an anion exchange chromatography column.

In one aspect, the quantitation comprises intact mass spectrometry analysis. In a specific aspect, a liquid chromatography column is connected inline to a mass spectrometer.

In one aspect, the variant featuring said at least one PTM is enriched in the flow-through relative to the control sample.

In one aspect, the method further comprises subjecting the flow-through to enzymatic digestion after step (b) and before step (c). In a specific aspect, the enzymatic digestion comprises contacting said flow-through to trypsin. In another specific aspect, the quantitation comprises peptide mapping mass spectrometry. In a more specific aspect, a liquid chromatography column is connected inline to a mass spectrometer.

In one aspect, the PTM is in a complementarity-determining region of an antigen-binding protein.

In one aspect, the method further comprises selecting a relative amount of protein of interest and capture molecule to contact in step (a), comprising: (a) contacting said sample including said protein of interest to said immobilized capture molecule; (b) collecting unbound protein of interest to produce a flow-through; (c) contacting said flow-through to a separation column to separate a variant of said protein of interest featuring a PTM known to reduce binding of said protein of interest to said capture molecule; (d) quantitating an amount of said variant; (e) comparing said amount to an amount quantitated in a control sample to determine an enrichment of said variant; (f) repeating steps (a)-(e) using varied relative amounts of said protein of interest to said capture molecule; and (g) comparing an enrichment of said variant across the relative amounts of step (f) to select a relative amount of protein of interest and capture molecule. In a specific aspect, the method further comprises determining a relative amount of bound to unbound protein of interest for each relative amount of protein of interest and capture molecule by measuring a concentration of bound protein of interest and unbound protein of interest using a UV detector.

In one aspect, a relative amount of protein of interest and capture molecule is selected to produce about 50% bound and about 50% unbound protein of interest. In another aspect, a relative amount of protein of interest and capture molecule is selected to product about 90% bound and about 10% unbound protein of interest.

This disclosure also provides a method for characterizing at least one post-translational modification (PTM) of a protein of interest. In some exemplary embodiments, the method comprises (a) contacting a sample including a protein of interest to a capture molecule, wherein said protein of interest features at least one PTM, said capture molecule is immobilized to a solid surface, and an amount of said capture molecule is insufficient to bind all of said protein of interest; (b) collecting unbound protein of interest to produce a flow-through; (c) contacting said flow-through to a separation column to separate at least one variant of said protein of interest featuring at least one PTM; (d) quantitating an amount of said at least one variant; (e) comparing said amount to an amount quantitated in a control sample; and (f) using said comparison to characterize said at least one PTM.

In one aspect, the protein of interest is selected from a group consisting of an antibody, an antigen-binding protein, a fusion protein, a receptor, a receptor ligand, and a therapeutic protein.

In one aspect, the PTM is in a complementarity-determining region of an antigen-binding protein.

In one aspect, the separation column is selected from a group consisting of a reverse phase chromatography column, normal phase chromatography column, hydrophobic interaction chromatography column, hydrophilic interaction chromatography column, ion exchange chromatography column, anion exchange chromatography column, cation exchange chromatography column, strong cation exchange chromatography column, and a weak cation exchange chromatography column.

In one aspect, the quantitation comprises intact mass spectrometry or peptide mapping mass spectrometry.

This disclosure further provides a method for identifying a variant of a protein of interest with reduced or increased binding to a capture molecule. In some exemplary embodiments, the method comprises (a) contacting a sample including a protein of interest to a capture molecule, wherein said sample includes at least one variant of said protein of interest, said capture molecule is immobilized to a solid surface, and an amount of said capture molecule is insufficient to bind all of said protein of interest; (b) collecting unbound protein of interest to produce a flow-through; (c) contacting said flow-through to a separation column to separate said at least one variant of said protein of interest; (d) quantitating an amount of said at least one variant; (e) comparing said amount to an amount quantitated in a control sample; and (f) using said comparison to identify a variant of said protein of interest with reduced or increased binding to said capture molecule.

In one aspect, the protein of interest is selected from a group consisting of an antibody, an antigen-binding protein, a fusion protein, a receptor, a receptor ligand, and a therapeutic protein.

In one aspect, the variant is selected from a group consisting of a post-translationally modified variant, a degraded variant, a truncated variant, an aggregated variant, and a misfolded variant of said protein of interest.

In one aspect, the separation column is selected from a group consisting of a reverse phase chromatography column, normal phase chromatography column, hydrophobic interaction chromatography column, hydrophilic interaction chromatography column, ion exchange chromatography column, anion exchange chromatography column, cation exchange chromatography column, strong cation exchange chromatography column, and a weak cation exchange chromatography column.

In one aspect, the quantitation comprises intact mass spectrometry or peptide mapping mass spectrometry.

This disclosure additionally provides methods for identifying at least one critical quality attribute (CQA) of a protein of interest. In some exemplary embodiments, the methods can comprise (a) contacting a sample including a protein of interest to a first target molecule, wherein said protein of interest binds to said first target molecule and said first target molecule is immobilized to a solid surface; (b) eluting said protein of interest from said solid surface to collect at least two fractions; (c) subjecting a first portion of said at least two fractions to surface plasmon resonance (SPR) analysis to characterize the binding of said protein of interest to a second target molecule; (d) subjecting a second portion of said at least two fractions to mass spectrometry (MS) analysis to identify variants of said protein of interest; and (e) comparing said variants to identify at least one CQA of said protein of interest.

In one aspect, said protein of interest is selected from a group consisting of an antibody, a monospecific antibody, a bispecific antibody, a trispecific antibody, an antibody-derived protein, an antigen-binding protein, a fusion protein, an Fc-fusion protein, a receptor, a receptor ligand, a therapeutic protein, a fragment thereof, and a combination thereof. In a specific aspect, said protein of interest is an antibody.

In one aspect, said first target molecule and/or said second target molecule is selected from a group consisting of an antibody, an antigen, a receptor, a receptor ligand, a therapeutic target, a fragment thereof, and a combination thereof. In a specific aspect, said target molecule is an antigen.

In one aspect, an amount of said first target molecule is insufficient to bind all of said protein of interest.

In one aspect, said solid surface is selected from a group consisting of a microplate, resin, beads, agarose beads, and magnetic beads.

In one aspect, said method further comprises immobilizing said first target molecule to said solid surface prior to step (a). In a specific aspect, said immobilizing comprises contacting a biotinylated first target molecule to a solid surface that is coated with avidin, streptavidin, or a variant thereof.

In one aspect, said eluting comprises contacting said solid surface to an elution buffer. In a specific aspect, a pH of said elution buffer is increased or decreased over time.

In one aspect, a number of said fractions is from 2 to 20, about 4, about 5, about 6, about 7, about 8, about 9, or about 10. In a specific aspect, a number of said fractions is 7.

In one aspect, said fractions comprise variants of said protein of interest with modified binding affinity to said first target molecule. In a specific aspect, fractions that elute earlier comprise species of said protein of interest with lower binding affinity compared to fractions that elute later.

In one aspect, said first target molecule is the same as said second target molecule.

In one aspect, the determination of a CQA is based on the binding of said protein of interest to said first target molecule and/or said second target molecule.

In one aspect, the method further comprises subjecting said at least two fractions to a digestion step prior to step (c). In a specific aspect, said digestion step comprises contacting each of said at least two fractions to at least one digestive enzyme. In a more specific aspect, said at least one digestive enzyme is selected from a group consisting of pepsin, trypsin, Tryp-N, chymotrypsin, Lys-N, Lys-C, Asp-N, Arg-C, Glu-C, papain, IdeS, variants thereof, and combinations thereof. In an even more specific aspect, said at least one digestive enzyme is IdeS or a variant thereof.

In one aspect, the method further comprises subjecting said at least two fractions to a separation step prior to MS analysis. In a specific aspect, said separation step comprises chromatography or electrophoresis. In a more specific aspect, said chromatography comprises reverse phase chromatography, normal phase chromatography, hydrophobic interaction chromatography, hydrophilic interaction chromatography, ion exchange chromatography, anion exchange chromatography, cation exchange chromatography, strong cation exchange chromatography, weak cation exchange chromatography, size exclusion chromatography, mixed-mode chromatography, or a combination thereof. In an even more specific aspect, said chromatography comprises size exclusion chromatography or strong cation exchange chromatography. In another specific aspect, said electrophoresis comprises capillary electrophoresis, isoelectric focusing, or imaged capillary isoelectric focusing.

In one aspect, said variants comprise acidic variants, basic variants, aggregates, crosslinking products, degradation products, truncation products, acylation, amidation, glycosylation, deglycosylation, oxidation, C-terminal lysine variation, N-terminal pyroglutamate variation, succinimide formation, iodination, acetylation, alkylation, methylation, adenylation, ADP-ribosylation, sulfonation, prenylation, hydroxylation, amidation, glutamylation, glycylation, isoprenylation, lipoylation, phosphopantetheinylation, phosphorylation, sulfation, citrullination, deamidation, SUMOylation, ubiquitination, glycation, glucuronylation, sialylation, or combinations thereof.

In one aspect, said at least one CQA comprises acidic variants, basic variants, aggregates, crosslinking products, degradation products, truncation products, acylation, amidation, glycosylation, deglycosylation, oxidation, C-terminal lysine variation, N-terminal pyroglutamate variation, succinimide formation, iodination, acetylation, alkylation, methylation, adenylation, ADP-ribosylation, sulfonation, prenylation, hydroxylation, amidation, glutamylation, glycylation, isoprenylation, lipoylation, phosphopantetheinylation, phosphorylation, sulfation, citrullination, deamidation, SUMOylation, ubiquitination, glycation, glucuronylation, sialylation, or combinations thereof.

In one aspect, a critical quality attribute is identified based on being an attribute of said protein of interest that is in higher abundance in a fraction that elutes earlier than another fraction. In another aspect, said at least one CQA is a post-translational modification. In a further aspect, said protein of interest is an antigen-binding protein and said at least one CQA is in a complementarity-determining region of said antigen-binding protein.

This disclosure further provides methods for characterizing binding variants of a protein of interest. In some exemplary embodiments, the methods can comprise (a) contacting a sample including a protein of interest to a first target molecule, wherein said protein of interest binds to said first target molecule and said first target molecule is immobilized to a solid surface; (b) eluting said protein of interest from said solid surface to collect at least two fractions; (c) subjecting a first portion of said at least two fractions to surface plasmon resonance (SPR) analysis to characterize the binding of said protein of interest to a second target molecule; (d) subjecting a second portion of each of said at least two fractions to separation by size or charge to produce a separation profile; and (e) comparing said binding and said separation profiles to characterize binding variants of said protein of interest.

In one aspect, said protein of interest is selected from a group consisting of an antibody, a monospecific antibody, a bispecific antibody, a trispecific antibody, an antibody-derived protein, an antigen-binding protein, a fusion protein, an Fc-fusion protein, a receptor, a receptor ligand, a therapeutic protein, a fragment thereof, and a combination thereof. In a specific aspect, said protein of interest is an antibody.

In one aspect, said first target molecule and/or said second target molecule is selected from a group consisting of an antibody, an antigen, a receptor, a receptor ligand, a therapeutic target, a fragment thereof, and a combination thereof. In a specific aspect, said first target molecule and/or said second target molecule is an antigen.

In one aspect, an amount of said first target molecule is insufficient to bind all of said protein of interest.

In one aspect, said solid surface is selected from a group consisting of a microplate, resin, beads, agarose beads, and magnetic beads.

In one aspect, said method further comprises immobilizing said first target molecule to said solid surface prior to step (a). In a specific aspect, said immobilizing comprises contacting a biotinylated first target molecule to a solid surface that is coated with avidin, streptavidin, or a variant thereof.

In one aspect, said eluting comprises contacting said solid surface to an elution buffer. In a specific aspect, a pH of said elution buffer is increased or decreased over time.

In one aspect, a number of said fractions is from 2 to 20, about 4, about 5, about 6, about 7, about 8, about 9, or about 10. In a specific aspect, a number of said fractions is 7.

In one aspect, said fractions comprise variants of said protein of interest with modified binding affinity to said first target molecule. In a specific aspect, fractions that elute earlier comprise species of said protein of interest with lower binding affinity compared to fractions that elute later.

In one aspect, said first target molecule is the same as said second target molecule.

In one aspect, the method further comprises subjecting said at least two fractions to a digestion step prior to step (c). In a specific aspect, said digestion step comprises contacting each of said at least two fractions to at least one digestive enzyme. In a more specific aspect, said at least one digestive enzyme is selected from a group consisting of pepsin, trypsin, Tryp-N, chymotrypsin, Lys-N, Lys-C, Asp-N, Arg-C, Glu-C, papain, IdeS, variants thereof, and combinations thereof. In an even more specific aspect, said at least one digestive enzyme is IdeS or a variant thereof.

In one aspect, the method further comprises subjecting said at least two fractions to a separation step prior to MS analysis. In a specific aspect, said separation step comprises chromatography or electrophoresis. In a more specific aspect, said chromatography comprises reverse phase chromatography, normal phase chromatography, hydrophobic interaction chromatography, hydrophilic interaction chromatography, ion exchange chromatography, anion exchange chromatography, cation exchange chromatography, strong cation exchange chromatography, weak cation exchange chromatography, size exclusion chromatography, mixed-mode chromatography, or a combination thereof. In an even more specific aspect, said chromatography comprises size exclusion chromatography or strong cation exchange chromatography. In another specific aspect, said electrophoresis comprises capillary electrophoresis, isoelectric focusing, or imaged capillary isoelectric focusing.

In one aspect, producing said separation profile comprises measuring said protein of interest using ultraviolet detection or fluorescence detection.

In one aspect, said binding variants comprise acidic variants, basic variants, aggregates, crosslinking products, degradation products, truncation products, acylation, amidation, glycosylation, deglycosylation, oxidation, C-terminal lysine variation, N-terminal pyroglutamate variation, succinimide formation, iodination, acetylation, alkylation, methylation, adenylation, ADP-ribosylation, sulfonation, prenylation, hydroxylation, amidation, glutamylation, glycylation, isoprenylation, lipoylation, phosphopantetheinylation, phosphorylation, sulfation, citrullination, deamidation, SUMOylation, ubiquitination, glycation, glucuronylation, sialylation, or combinations thereof.

In one aspect, the method further comprises subjecting said separated fractions to MS analysis to quantify, characterize, and/or identify said binding variants.

In one aspect, said binding variant has reduced binding compared to a main species of said protein of interest.

These, and other, aspects of the present invention will be better appreciated and understood when considered in conjunction with the following description and accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1 is a representation of possible different product-related variants of an antibody including size variants, charge variants, and post-translational modifications (PTMs).

FIG. 2 is a representation of methods routinely used to determine or monitor CQAs during protein drug development.

FIG. 3A shows a method design and workflow for identifying at least one product-related variant, according to an exemplary embodiment.

FIG. 3B shows a method for identifying at least one product-related variant, according to an exemplary embodiment.

FIG. 4 shows a method design and workflow to determine the antigen to antibody ratio, according to an exemplary embodiment.

FIG. 5A shows a titration curve obtained to determine a percentage of bispecific antibody flow-through in a competitive binding assay based on an antigen to antibody ratio, according to an exemplary embodiment.

FIG. 5B shows a titration curve obtained to determine a percentage of monospecific antibody flow-through in a competitive binding assay based on an antigen to antibody ratio, according to an exemplary embodiment.

FIG. 6A shows an enrichment of a binding-related modification of a bsAb using competitive binding conditions, according to an exemplary embodiment.

FIG. 6B shows an enrichment of a binding-related modification of a msAb using competitive binding conditions, according to an exemplary embodiment.

FIG. 7A shows a total ion chromatogram (TIC) of an unfractionated bsAb-1 sample and extracted ion chromatograms (XIC) for each modified variant, according to an exemplary embodiment.

FIG. 7B shows a TIC of flow-through from a competitive binding assay on a bsAb-1 sample and XICs for each modified variant, according to an exemplary embodiment.

FIG. 7C shows a relative abundance of each bsAb-1 attribute from an unfractionated sample compared to a flow-through from a competitive binding assay, according to an exemplary embodiment.

FIG. 8 shows a structure of bsAb-2, according to an exemplary embodiment.

FIG. 9A shows a total ion chromatogram (TIC) of an unfractionated bsAb-2 sample and extracted ion chromatograms (XIC) for each modified variant, according to an exemplary embodiment.

FIG. 9B shows a TIC of flow-through from a competitive binding assay on a bsAb-2 sample and XICs for each modified variant, according to an exemplary embodiment.

FIG. 9C shows a relative abundance of each bsAb-2 attribute from an unfractionated sample compared to a flow-through from a competitive binding assay, according to an exemplary embodiment.

FIG. 10A shows a total ion chromatogram (TIC) of an unfractionated msAb-2 sample and extracted ion chromatograms (XIC) for each modified variant, according to an exemplary embodiment.

FIG. 10B shows a TIC of flow-through from a competitive binding assay on a msAb-2 sample and XICs for each modified variant, according to an exemplary embodiment.

FIG. 10C shows a relative abundance of each msAb-2 attribute from an unfractionated sample compared to a flow-through from a competitive binding assay, according to an exemplary embodiment.

FIG. 11 shows a quantification of each bsAb-2 attribute using bottom-up RP-LC/MS analysis, according to an exemplary embodiment.

FIG. 12A shows a percent increase in relative abundance of each bsAb-1 attribute from an unfractionated sample compared to a flow-through from a competitive binding assay plotted against a p value from Student's t-test of three replicates using bottom-up RP-LC/MS analysis, according to an exemplary embodiment.

FIG. 12B shows a percent increase in relative abundance of each bsAb-2 attribute from an unfractionated sample compared to a flow-through from a competitive binding assay plotted against a p value from Student's t-test of three replicates using bottom-up RP-LC/MS analysis, according to an exemplary embodiment.

FIG. 12C shows a percent increase in relative abundance of each msAb-2 attribute from an unfractionated sample compared to a flow-through from a competitive binding assay plotted against a p value from Student's t-test of three replicates using bottom-up RP-LC/MS analysis, according to an exemplary embodiment.

FIG. 13A illustrates an affinity fractionation method for pCQA identification, according to an exemplary embodiment.

FIG. 13B illustrates the fractionation of a protein sample using affinity enrichment, according to an exemplary embodiment.

FIG. 14A illustrates a biolayer interferometry (BLI) assay format for assessing the affinity of a protein of interest for its target, according to an exemplary embodiment.

FIG. 14B shows the target-binding affinity of msAb-3 from each affinity enrichment fraction as measured using BLI, according to an exemplary embodiment.

FIG. 15A illustrates a cell-based bioassay format for characterizing therapeutic protein potency, according to an exemplary embodiment.

FIG. 15B shows a dose response curve for a cell-based bioassay, according to an exemplary embodiment.

FIG. 15C illustrates a cell-based bioassay format for characterizing therapeutic protein potency, according to an exemplary embodiment.

FIG. 15D shows an inhibition curve for a cell-based bioassay, according to an exemplary embodiment.

FIG. 16 shows the potency of msAb-3 from each affinity enrichment fraction as measured by a cell-based bioassay, according to an exemplary embodiment.

FIG. 17A shows a summary of surface plasmon resonance (SPR) analysis of msAb-3 from each affinity enrichment fraction binding to its target, according to an exemplary embodiment.

FIG. 17B shows SPR sensorgrams of msAb-3 from each affinity enrichment binding to its target, according to an exemplary embodiment.

FIG. 18 shows the separation of size variants for each affinity enrichment fraction of msAb-3 using size exclusion-ultra performance liquid chromatography (SE-UPLC), according to an exemplary embodiment.

FIG. 19 shows the separation of size and charge variants for each affinity enrichment fraction of msAb-3 using non-reducing microchip electrophoresis (MCE-SDS), according to an exemplary embodiment.

FIG. 20 shows the separation of size and charge variants for each affinity enrichment fraction of msAb-3 using reducing MCE-SDS, according to an exemplary embodiment.

FIG. 21 shows the separation of charge variants for each affinity enrichment fraction of msAb-3 using native imaged capillary isoelectric focusing (iCIEF), according to an exemplary embodiment.

FIG. 22 shows the separation of charge variants for each affinity enrichment fraction of msAb-3 using native strong cation exchange chromatography coupled with ultraviolet detection and mass spectrometry (SCX-UV-MS), according to an exemplary embodiment.

FIG. 23 shows charge variants identified for each affinity enrichment fraction of msAb-3 using SCX-UV-MS, according to an exemplary embodiment.

FIG. 24A shows mass spectrometry (MS) analysis of the main charge species from msAb-3 drug substance (DS), according to an exemplary embodiment.

FIG. 24B shows MS analysis of the A4 peak from msAb-3 DS, according to an exemplary embodiment.

FIG. 24C shows MS analysis of the A3 peak from msAb-3 DS, according to an exemplary embodiment.

FIG. 24D shows MS analysis of the A2 peak from msAb-3 DS, according to an exemplary embodiment.

FIG. 24E shows MS analysis of the A1 peak from msAb-3 DS, according to an exemplary embodiment.

FIG. 24F shows MS analysis of the B1 peak from msAb-3 DS, according to an exemplary embodiment.

FIG. 24G shows MS analysis of the B2 peak from msAb-3 DS, according to an exemplary embodiment.

FIG. 24H shows MS analysis of the B3 peak from msAb-3 DS, according to an exemplary embodiment.

FIG. 24I shows MS analysis of the B4 peak from msAb-3 DS, according to an exemplary embodiment.

FIG. 25A shows charge variants identified for affinity enrichment fraction 1 of msAb-3 using SCX-UV, according to an exemplary embodiment.

FIG. 25B shows MS analysis of the main charge peak for affinity enrichment fraction 1 of msAb-3, according to an exemplary embodiment.

FIG. 25C shows MS analyses of peaks P1-P4 for affinity enrichment fraction 1 of msAb-3, according to an exemplary embodiment.

FIG. 25D shows MS analyses of peaks P5-P8 for affinity enrichment fraction 1 of msAb-3, according to an exemplary embodiment.

FIG. 25E shows MS analyses of peaks P9-P12 for affinity enrichment fraction 1 of msAb-3, according to an exemplary embodiment.

FIG. 25F shows MS analyses of peaks P13-P16 for affinity enrichment fraction 1 of msAb-3, according to an exemplary embodiment.

FIG. 25G shows MS analyses of peaks P17-P20 for affinity enrichment fraction 1 of msAb-3, according to an exemplary embodiment.

FIG. 26 illustrates a workflow for subunit analysis of an antibody of interest, according to an exemplary embodiment.

FIG. 27A shows the separation of charge variants for each affinity enrichment fraction of msAb-3 using IdeS digestion and native SCX-UV-MS, according to an exemplary embodiment.

FIG. 27B shows MS analyses of the main charge peak and peaks A1 and A2 from IdeS digestion and SCX-UV-MS of msAb-3 drug substance, according to an exemplary embodiment.

FIG. 27C shows MS analyses of the main charge peak and peaks A3 and A4 from IdeS digestion and SCX-UV-MS of msAb-3 drug substance, according to an exemplary embodiment.

FIG. 27D shows MS analyses of peaks B1-B3 from IdeS digestion and SCX-UV-MS of msAb-3 drug substance, according to an exemplary embodiment.

FIG. 28 shows a comparison of relative Fab fragment charge variant peak abundances between affinity enrichment fractions of msAb-3, according to an exemplary embodiment.

FIG. 29A shows MS analyses of peak A4 from IdeS digestion and SCX-UV-MS of affinity fractions of msAb-3, according to an exemplary embodiment.

FIG. 29B shows MS analyses of peak A3 from IdeS digestion and SCX-UV-MS of affinity fractions of msAb-3, according to an exemplary embodiment.

FIG. 29C shows MS analyses of peak A2 from IdeS digestion and SCX-UV-MS of affinity fractions of msAb-3, according to an exemplary embodiment.

FIG. 29D shows MS analyses of peak A1 from IdeS digestion and SCX-UV-MS of affinity fractions of msAb-3, according to an exemplary embodiment.

### DETAILED DESCRIPTION

Development of therapeutic monoclonal antibodies (mAbs) remains a challenging process despite its huge success in the last two decades (Torre et al., 2021, Molecules, 26:627). Due to their large size and complexity, mAb molecules often host a large number of modifications (e.g., attributes) that need to be thoroughly characterized to support their development (Krishnan and Darling, 2020, Drug Discovery, 2020:31; Rogers et al., 2018, AAPS, 20:1-8). These attributes can be introduced from both post-translational modifications (e.g., deamidation, oxidation and glycation) (Jefferis, 2016, Journal of Immunology Research; Jenkins et al., 2008, Molecular Biotechnology, 39:113-8; Moritz and Stracke, 2017, Electrophoresis, 38:769-85; Wang et al., 2018, Biotechnol. Bioeng., 115:1378-93) and physicochemical degradations (e.g., aggregation and fragmentation) during mAb production, purification, and storage (Wang et al., 2007, Journal of Pharmaceutical Sciences, 96:1-26). Of these attributes, those that can impact the efficacy or safety of the drug products are defined as critical quality attributes (CQAs) (Beyer et al., 2018, Biotechnology Journal, 13:1700476; Hmiel et al., 2015, Analytical and Bioanalytical Chemistry, 407:79-94; Li et al., 2015, ACS Publications, 2:119-83). Identification and quantification of product-related variants in biologic products can be very important during the production and development of a product. The identification of such variants can be imperative for developing a safe and effective product. Hence, a robust method and/or workflow to characterize CQAs can be beneficial.

The Annex to ICH Q8 defines CQAs as physical, chemical, biological or microbiological properties or characteristics that should be within an appropriate limit, range or distribution to ensure the desired product quality, safety/immunogenicity, efficacy and pharmacodynamics/pharmacokinetics (US Food and Drug Administration. Guidance for industry: Q8(R2) pharmaceutical development. fda.gov/media/71535/download). Thus, CQAs must be within an appropriate limit, range or distribution to ensure the desired product quality, safety and efficacy. For example, for monoclonal antibody therapeutics that rely on fragment crystallizable (Fc)-mediated effector function for their clinical activity, the terminal sugars of Fc glycans have been shown to be critical for safety or efficacy.

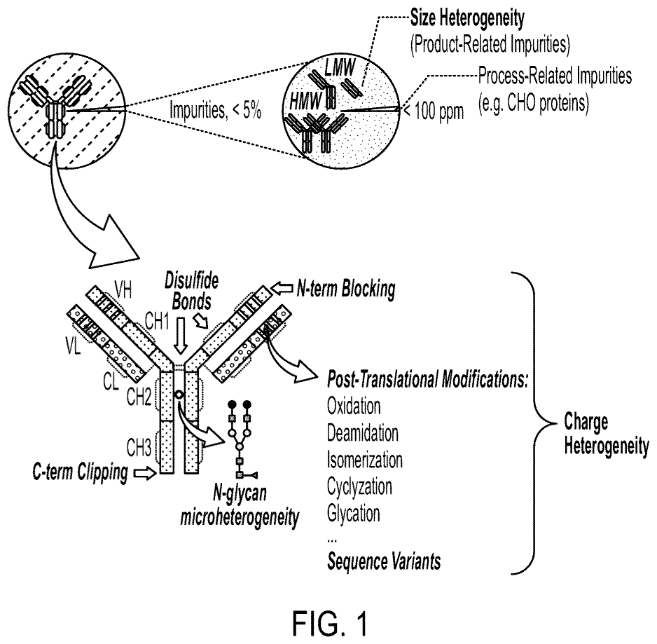

FIG. 1 shows a non-limiting example of variants that can be critical quality attributes for a protein. In case of the antibody represented in FIG. 1, variants can be size variants like low molecular weight (LMW) fragmentation products and high molecular weight (HMW) aggregation products. Other product-related impurities can be charge variants formed due to N-terminal blocking, disulfide bond formation, C-terminal clipping, Fc glycan microheterogeneity, or other post-translational modifications. These can cause decreased binding of the protein of interest and need to be monitored at various parts of the manufacturing and delivery process.

Frequently, CQAs that impair a mAb's target binding affinity are found within the mAb complementarity-determining regions (CDRs), although they could also occur on residues outside the CDRs through allosteric effects. Conversely, some CDR modifications might not be considered as CQAs if they are not directly or indirectly affecting the epitope-paratope interactions (Yan et al., 2016, Anal. Chem., 88:2041-50). Therefore, in addition to empirical knowledge or computational modelling approaches, it is also critical to experimentally assess each CDR modification for its impact on mAb target binding.

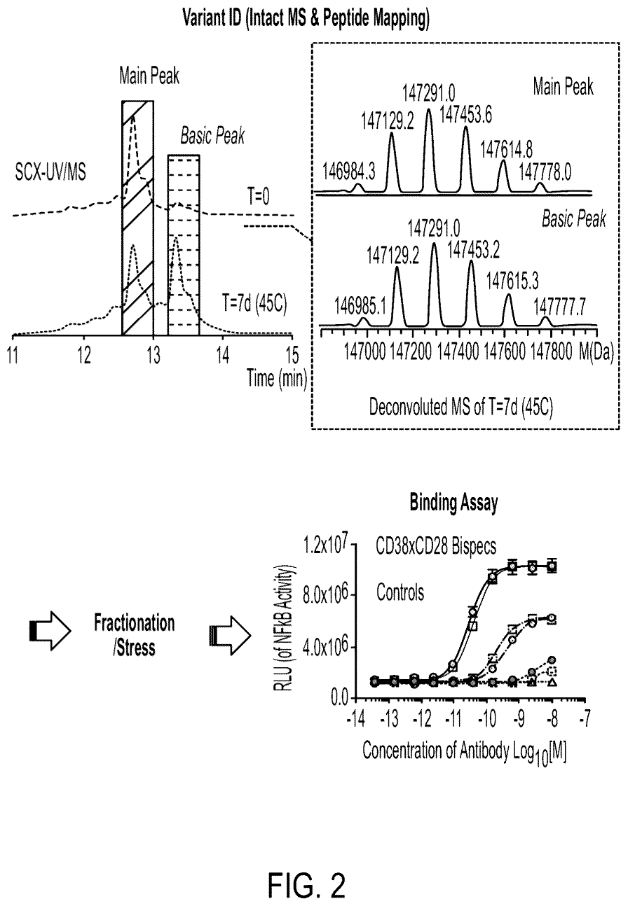

Identification of potential CQAs (pCQAs) that impact mAb target binding is particularly important during drug candidate developability assessment, which is a vital step to select drug candidates with favorable drug-like properties, and therefore, reduce failure rates (Saitoh, 2018, Journal of the Pharmaceutical Society of Japan, 138:1475-81; Fogel, 2018, Contemporary Clinical Trials Communications, 11:156-64). Currently, this task is performed in a low-throughput fashion that requires enrichment of the attribute-bearing variants followed by either in vitro target binding measurement or cell-based potency testing (Geigert, 2013, Springer). For example, enrichment of mAb variants can be achieved through liquid chromatography (LC) fractionation under different separation modes, such as ion exchange chromatography (IEX) (Yan et al., 2018, Analytical Chemistry, 90:13013-20; Zhang et al., 2008, Analytical Chemistry, 80:7022-8), hydrophobic interaction chromatography (HIC) (Fekete et al., 2016, Journal of Pharmaceutical and Biomedical Analysis, 130:3-18; Wang et al., 2018, Journal of Pharmaceutical and Biomedical Analysis, 154:468-75), and size exclusion chromatography (SEC) (Kukrer et al., 2010, Pharmaceutical Research, 27:2197-204; Lu et al., 2013, MAbs, 5(1):102-13). One of the conventional methods includes use of strong cation exchange chromatography (SCX). One such workflow is shown in FIG. 2. This includes performing separation of the protein of interest and its variants by SCX followed by conducting a binding assay of the protein of interest and its variant to identify if the variant has reduced binding affinity compared to the protein of interest.

This approach, besides being highly laborious, is challenging for isolating low-abundance variants. Therefore, specific stress conditions might also be required to artificially generate the variants to higher levels for the subsequent assessment (Thiagarajan et al., 2015, Journal of Raman Spectroscopy, 46:531-6). Furthermore, to isolate the variants with sufficient purity, significant efforts are often needed to optimize an LC method. Finally, as each attribute needs to be evaluated one at a time, this approach is low-throughput, and therefore, not ideally suited during candidate developability assessment, where multiple candidates might need to be evaluated simultaneously with fast turn-around times.

To address these limitations, size exclusion chromatography (SEC) has recently been utilized as an alternative means to enrich mAb variants with compromised target binding affinity. Following mAb-antigen incubation, the unbound mAb species were separated from the mAb-antigen complexes by SEC. Subsequent bottom-up MS analysis of these SEC fractions could identify attributes that were enriched in the unbound fraction due to impaired antigen binding (Bondarenko et al., 2021, MAbs, 13:1887629). Later, Shi et al. (Shi et al., 2021, MAbs, 13:1887612) further incorporated a competitive binding step to this workflow and showed improved method sensitivity in identifying attributes with less significant impact on target binding. This SEC fractionation and bottom-up MS-based approach significantly improved the method throughput in pCQA evaluation. However, due to the needs for SEC fractionation, this method still required the mAb variants to be present at sufficiently high levels. As a result, forced degradation conditions were often needed in these studies to increase the abundances of the variants prior to the SEC fractionation. Moreover, as this approach requires at least partial resolution between the unbound mAb and the mAb-antigen complexes from SEC separation, it may not be suitable for systems where the antigen is too small to result in a meaningful SEC retention time shift upon binding (e.g., small cytokines) or the binding stoichiometry is too complicated to generate discrete complexes (e.g., multivalent antigens might form heterogeneous mAb-antigen complexes with different stoichiometries).

This disclosure sets forth affinity enrichment methods for enriching product variants, characterizing microheterogeneity in drug products, and understanding the impact of variants on structure, functional and biophysical properties, potency, and potential immunogenicity. Affinity enrichment can be performed during early drug development, for example immediately prior to or at the start of pre-clinical development following the discovery phase and candidate selection, to identify potential PTM hot spots. One advantage of affinity enrichment is that the same target used for immunization and candidate selection can be leveraged. Identified PTMs and hot spots can be connected to analytics that can be used for monitoring during manufacturing development.

This disclosure sets forth a novel, high-throughput method for characterizing binding-related CQAs in a protein of interest. The challenges described above were overcome through the development a novel workflow, namely, competitive binding mass spectrometry (MS), to enable high-throughput evaluation of target binding-related pCQAs in a protein of interest, for example therapeutic mAbs. By performing the competitive binding on immobilized antigen, mAb variants with impaired target binding can be effectively enriched in the unbound fraction. Comparing to SEC fractionation, the use of immobilized antigen for variant enrichment not only simplifies the experimental procedures, but also allows the new workflow to be applicable to a much broader mAb-antigen system. Following mAb variants enrichment, the criticality of multiple attributes can then be simultaneously assessed by comparing their relative abundances between the unfractionated control sample and the unbound fraction using quantitative mass spectrometry approaches. Finally, attributes that showed significant enrichment in the unbound fraction were determined as pCQAs, as they resulted in a decreased target binding affinity. The validity and utility of this new method was demonstrated in three mAb case studies, where a wide range of CDR and non-CDR modifications were assessed for their impact on target binding.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described. All publications mentioned are hereby incorporated by reference.

The term “a” should be understood to mean “at least one”; and the terms “about” and “approximately” should be understood to permit standard variation as would be understood by those of ordinary skill in the art; and where ranges are provided, endpoints are included.

In some exemplary embodiments, the disclosure provides a method identifying at least one product-related variant in a sample comprising a protein of interest.

As used herein, the term “protein” or “protein of interest” includes any amino acid polymer having covalently linked amide bonds. Proteins comprise one or more amino acid polymer chains, generally known in the art as “polypeptides”. “Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. “Synthetic peptides or polypeptides' refers to a non-naturally occurring peptide or polypeptide. Synthetic peptides or polypeptides can be synthesized, for example, using an automated polypeptide synthesizer. Various solid phase peptide synthesis methods are known. A protein may contain one or multiple polypeptides to form a single functioning biomolecule. A protein can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. In another exemplary aspect, a protein can include antibody fragments, nanobodies, recombinant antibody chimeras, cytokines, chemokines, peptide hormones, and the like. Proteins may be produced using recombinant cell-based production systems, such as the insect bacculovirus system, yeast systems (e.g., Pichia sp.), mammalian systems (e.g., CHO cells and CHO derivatives like CHO-K1 cells). For a review discussing biotherapeutic proteins and their production, see Ghaderi et al., “Production platforms for biotherapeutic glycoproteins. Occurrence, impact, and challenges of non-human sialylation,” (BIOTECHNOL. GENET. ENG. REV. 147-175 (2012)). In some exemplary embodiments, proteins comprise modifications, adducts, and other covalently linked moieties. Those modifications, adducts and moieties include for example avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

In some exemplary embodiments, the protein can be an antibody, a bispecific antibody, a multispecific antibody, antibody fragment, monoclonal antibody, an Fc fusion protein, an antigen-binding protein, a receptor, a receptor ligand, or a therapeutic protein.

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L1}). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different exemplary embodiments, the FRs of the anti-big-ET-1 antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a Fc fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. An antibody fragment may be produced by various means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains that are linked together, for example, by disulfide linkages. An antibody fragment may optionally comprise a multi-molecular complex.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

The term “Fc fusion proteins” as used herein includes part or all of two or more proteins, one of which is an Fc portion of an immunoglobulin molecule, that are not fused in their natural state. Preparation of fusion proteins comprising certain heterologous polypeptides fused to various portions of antibody-derived polypeptides (including the Fc domain) has been described, e.g., by Ashkenazi et al., Proc. Natl. Acad. ScL USA 88: 10535, 1991; Byrn et al., Nature 344:677, 1990; and Hollenbaugh et al., “Construction of Immunoglobulin Fusion Proteins”, in Current Protocols in Immunology, Suppl. 4, pages 10.19.1-10.19.11, 1992. “Receptor Fc fusion proteins” comprise one or more of one or more extracellular domain(s) of a receptor coupled to an Fc moiety, which in some embodiments comprises a hinge region followed by a CH2 and CH3 domain of an immunoglobulin. In some embodiments, the Fc-fusion protein contains two or more distinct receptor chains that bind to a single or more than one ligand(s). For example, an Fc-fusion protein is a trap, such as for example an IL-1 trap (e.g., Rilonacept, which contains the IL-1 RAcP ligand binding region fused to the IL-1R1 extracellular region fused to Fc of hIgG1; see U.S. Pat. No. 6,927,004, which is herein incorporated by reference in its entirety), or a VEGF Trap (e.g., Aflibercept, which contains the Ig domain 2 of the VEGF receptor Flt1 fused to the Ig domain 3 of the VEGF receptor Flk1 fused to Fc of hIgG1; e.g., SEQ ID NO:1; see U.S. Pat. Nos. 7,087,411 and 7,279,159, which are herein incorporated by reference in their entirety).

As used herein, the term “target” refers to any molecule that may specifically interact with a therapeutic protein in order to achieve a pharmacological effect. For example, the target of an antibody may be an antigen against which it is directed; the target of a ligand may be a receptor to which it preferentially binds, and vice versa; the target of an enzyme may be a substrate to which it preferentially binds; and so forth. A single therapeutic protein may have more than one target. A variety of targets are suitable for use in the method of the invention, according to the specific application. A target may, for example, be present on a cell surface, may be soluble, may be cytosolic, or may be immobilized on a solid surface. A target may be recombinant protein. In some exemplary embodiments, the target may be an antigen.

As used herein, the term “capture molecule” refers to any molecule that may be used to capture a protein of interest. A capture molecule may be immobilized to a solid surface, for example through a biotin-streptavidin interaction. A capture molecule may also be a target molecule for a therapeutic protein. For example, a capture molecule for an antigen-binding protein such as an antibody may be an antigen against which it is directed, and vice versa; a capture molecule for an antigen-binding protein may also be a heavy-chain binding molecule such as Protein A; a capture molecule for a ligand may be a receptor to which it specifically binds, and vice versa; a capture molecule for an enzyme may be a substrate to which is specifically binds, and vice versa; and so forth. A protein of interest may have several suitable capture molecules, one or more of which may be selected by a person of skill in the art.

As used herein, the term “impurity” can include any undesirable protein present in the protein biopharmaceutical product. Impurity can include process and product-related impurities. The impurity can further be of known structure, partially characterized, or unidentified.

Process-related impurities can be derived from the manufacturing process and can include the three major categories: cell substrate-derived, cell culture-derived and downstream derived. Cell substrate-derived impurities include, but are not limited to, proteins derived from the host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables.

Product-related impurities (e.g., precursors, certain degradation products) can be molecular variants arising during manufacture and/or storage that do not have properties comparable to those of the desired product with respect to activity, efficacy, and safety. Such variants may need considerable effort in isolation and characterization in order to identify the type of modification(s). Product-related impurities can include truncated forms, modified forms, and aggregates. Truncated forms are formed by hydrolytic enzymes or chemicals which catalyze the cleavage of peptide bonds. Modified forms include, but are not limited to, deamidated, isomerized, mismatched S—S linked, oxidized, or altered conjugated forms (e.g., glycosylation, phosphorylation). Modified forms can also include any post-translationally modified form. Aggregates include dimers and higher multiples of the desired product. (Q6B Specifications: Test Procedures and Acceptance Criteria for Biotechnological/Biological Products, ICH August 1999, U.S. Dept. of Health and Humans Services).

Some product-related impurities or product-related protein variants have compromised binding affinity. Compromised binding affinity, here, includes a reduced binding affinity to the target of the protein of interest or an antigen designed for the protein of interest. The compromised binding affinity can be any affinity which is less than the affinity of the protein of interest towards the target of the protein of interest or an antigen designed for the protein of interest. Conversely, a variant of a protein of interest may have relatively increased binding to a particular capture molecule.

As used herein, the general term “post-translational modifications” or “PTMs” refers to covalent modifications that polypeptides undergo, either during (co-translational modification) or after (post-translational modification) their ribosomal synthesis. PTMs are generally introduced by specific enzymes or enzyme pathways. Many occur at the site of a specific characteristic protein sequence (signature sequence) within the protein backbone. Several hundred PTMs have been recorded, and these modifications invariably influence some aspect of a protein's structure or function (Walsh, G. “Proteins” (2014) second edition, published by Wiley and Sons, Ltd., ISBN: 9780470669853). The various post-translational modifications include, but are not limited to, cleavage, N-terminal extensions, protein degradation, acylation of the N-terminus, biotinylation (acylation of lysine residues with a biotin), amidation of the C-terminal, glycosylation, iodination, covalent attachment of prosthetic groups, acetylation (the addition of an acetyl group, usually at the N-terminus of the protein), alkylation (the addition of an alkyl group (e.g. methyl, ethyl, propyl) usually at lysine or arginine residues), methylation, adenylation, ADP-ribosylation, covalent cross links within, or between, polypeptide chains, sulfonation, prenylation, Vitamin C dependent modifications (proline and lysine hydroxylations and carboxy terminal amidation), Vitamin K dependent modification wherein Vitamin K is a cofactor in the carboxylation of glutamic acid residues resulting in the formation of a γ-carboxyglutamate (a glu residue), glutamylation (covalent linkage of glutamic acid residues), glycylation (covalent linkage glycine residues), glycosylation (addition of a glycosyl group to either asparagine, hydroxylysine, serine, or threonine, resulting in a glycoprotein), isoprenylation (addition of an isoprenoid group such as farnesol and geranylgeraniol), lipoylation (attachment of a lipoate functionality), phosphopantetheinylation (addition of a 4′-phosphopantetheinyl moiety from coenzyme A, as in fatty acid, polyketide, non-ribosomal peptide and leucine biosynthesis), phosphorylation (addition of a phosphate group, usually to serine, tyrosine, threonine or histidine), and sulfation (addition of a sulfate group, usually to a tyrosine residue). The post-translational modifications that change the chemical nature of amino acids include, but are not limited to, citrullination (the conversion of arginine to citrulline by deimination), and deamidation (the conversion of glutamine to glutamic acid or asparagine to aspartic acid). The post-translational modifications that involve structural changes include, but are not limited to, formation of disulfide bridges (covalent linkage of two cysteine amino acids) and proteolytic cleavage (cleavage of a protein at a peptide bond). Certain post-translational modifications involve the addition of other proteins or peptides, such as ISGylation (covalent linkage to the ISG15 protein (Interferon-Stimulated Gene)), SUMOylation (covalent linkage to the SUMO protein (Small Ubiquitin-related MOdifier)) and ubiquitination (covalent linkage to the protein ubiquitin). See European Bioinformatics Institute Protein Information ResourceSIB Swiss Institute of Bioinformatics, EUROPEAN BIOINFORMATICS INSTITUTE DRS—DROSOMYCIN PRECURSOR—DROSOPHILA MELANOGASTER (FRUIT FLY)—DRS GENE & PROTEIN, http://www.uniprot.org/docs/ptmlist (last visited Jan. 15, 2019) for a more detailed controlled vocabulary of PTMs curated by UniProt.

As used herein, the term “separation column” refers to any column to which a sample may be applied to facilitate separation of components of the sample. A separation column can be, for example, a solid phase extraction column or a chromatography column.

As used herein, the term “chromatography” refers to a process in which a chemical mixture carried by a liquid or gas can be separated into components as a result of differential distribution of the chemical entities as they flow around or over a stationary liquid or solid phase. Non-limiting examples of chromatography include traditional reversed-phased (RP), ion exchange (IEX), mixed mode chromatography and normal phase chromatography (NP).

As used herein, the term “cation exchange chromatography” means a chromatography method which uses a “cation exchange chromatography material”. Further depending on the nature of the charged group the “cation exchange chromatography material” is referred to as e.g. in the case of cation exchange chromatography materials with sulfonic acid groups (S), or carboxymethyl groups (CM). Depending on the chemical nature of the charged group the “cation exchange chromatography material” can additionally be classified as strong or weak ion exchange chromatography material, depending on the strength of the covalently bound charged substituent. For example, strong cation exchange chromatography materials have a sulfonic acid group as chromatographic functional group.

For example, “cation exchange chromatography materials”, for example, are available under different names from a multitude of companies such as e.g. Bio-Rex, Macro-Prep CM (available from BioRad Laboratories, Hercules, Calif., USA), weak cation exchanger WCX 2 (available from Ciphergen, Fremont, Calif, USA), Dowex MAC-3 (available from Dow chemical company, Midland, Mich., USA), Mustang C (available from Pall Corporation, East Hills, N.Y., USA), Cellulose CM-23, CM-32, CM-52, hyper-D, and partisphere (available from Whatman plc, Brentford, UK), Amberlite IRC 76, IRC 747, IRC 748, GT 73 (available from Tosoh Bioscience GmbH, Stuttgart, Germany), CM 1500, CM 3000 (available from BioChrom Labs, Terre Haute, Ind., USA), and CM-Sepharose Fast Flow (available from GE Healthcare, Life Sciences, Germany). In addition, commercially available cation exchange resins further include carboxymethyl-cellulose, Bakerbond ABX, sulphopropyl (SP) immobilized on agarose (e.g. SP-Sepharose Fast Flow or SP-Sepharose High Performance, available from GE Healthcare-Amersham Biosciences Europe GmbH, Freiburg, Germany) and sulphonyl immobilized on agarose (e.g. S-Sepharose Fast Flow available from GE Healthcare, Life Sciences, Germany).

The “cation exchange chromatography materials” include mixed-mode chromatography materials performing a combination of ion exchange and hydrophobic interaction technologies (e.g., Capto adhere, Capto MMC, MEP HyperCell, Eshmuno HCX, etc.), mixed-mode chromatography material s performing a combination of anion exchange and cation exchange technologies (e.g., hydroxyapatite, ceramic hydroxyapatite, etc.), and the like. Cation exchange chromatography materials that may be used in cation exchange chromatography in the present invention may include, but are not limited to, all the commercially available cation exchange chromatography materials as described above. In an example of the present invention YMC BioPro SP-F column was used as cation exchange chromatography material.

As used herein, the term “mass spectrometer” includes a device capable of identifying specific molecular species and measuring their accurate masses. The term is meant to include any molecular detector into which a polypeptide or peptide may be eluted for detection and/or characterization. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization). The choice of ion source depends heavily on the application.

In some embodiments, the mass spectrometer can be an electrospray-mass spectrometer. As used herein, the term “electrospray ionization” or “ESI” refers to the process of spray ionization in which either cations or anions in solution are transferred to the gas phase via formation and desolvation at atmospheric pressure of a stream of highly charged droplets that result from applying a potential difference between the tip of the electrospray needle containing the solution and a counter electrode. There are generally three major steps in the production of gas-phase ions from electrolyte ions in solution. These are: (a) production of charged droplets at the ES infusion tip; (b) shrinkage of charged droplets by solvent evaporation and repeated droplet disintegrations leading to small highly charged droplets capable of producing gas-phase ions; and (c) the mechanism by which gas-phase ions are produced from very small and highly charged droplets. Stages (a)-(c) generally occur in the atmospheric pressure region of the apparatus.

As used herein, the term “electrospray infusion setup” refers to an electrospray ionization system that is compatible with a mass spectrometer used for mass analysis of protein. In electrospray ionization, an electrospray needle has its orifice positioned close to the entrance orifice of a spectrometer. A sample, containing the protein of interest, can be pumped through the syringe needle. An electric potential between the syringe needle orifice and an orifice leading to the mass analyzer forms a spray (“electrospray”) of the solution. The electrospray can be carried out at atmospheric pressure and provides highly charged droplets of the solution. The electrospray infusion setup can include an electrospray emitter, nebulization gas, and/or an ESI power supply. The setup can optionally be automated to carry out sample aspiration, sample dispensing, sample delivery, and/or for spraying the sample.

In some exemplary embodiments, the electrospray ionization mass spectrometer can be a nano-electrospray ionization mass spectrometer. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

As used herein, the term “mass analyzer” includes a device that can separate species, that is, atoms, molecules, or clusters, according to their mass. Non-limiting examples of mass analyzers that could be employed for fast protein sequencing are time-of-flight (TOF), magnetic/electric sector, quadrupole mass filter (Q), quadrupole ion trap (QIT), orbitrap, Fourier transform ion cyclotron resonance (FTICR), and also the technique of accelerator mass spectrometry (AMS).

In some exemplary embodiments, mass spectrometry can be performed under native conditions. As used herein, the term “native conditions” or “native MS” or “native ESI-MS” can include a performing mass spectrometry under conditions that preserve no-covalent interactions in an analyte. For detailed review on native MS, refer to the review: Elisabetta Boeri Erba & Carlo Petosa, The emerging role of native mass spectrometry in characterizing the structure and dynamics of macromolecular complexes, 24 PROTEIN SCIENCE 1176-1192 (2015).

In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules can be transferred into gas phase and ionized intact and that they can be induced to fall apart in some predictable and controllable fashion after the first mass selection step. Multistage MS/MS, or MSⁿ, can be performed by first selecting and isolating a precursor ion (MS²), fragmenting it, isolating a primary fragment ion (MS³), fragmenting it, isolating a secondary fragment (MS⁴), and so on as long as one can obtain meaningful information or the fragment ion signal is detectable. Tandem MS have been successfully performed with a wide variety of analyzer combinations. What analyzers to combine for a certain application is determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time mass spectrometer ions produced in the ion source can be trapped, isolated, fragmented, and m/z separated in the same physical device.

The peptides identified by the mass spectrometer can be used as surrogate representatives of the intact protein and their post-translational modifications. They can be used for protein characterization by correlating experimental and theoretical MS/MS data, the latter generated from possible peptides in a protein sequence database. The characterization can include, but is not limited, to sequencing amino acids of the protein fragments, determining protein sequencing, determining protein de novo sequencing, locating post-translational modifications, or identifying post translational modifications, or comparability analysis, or combinations thereof.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com//proteinpilot), Phenyx (www.phenyx-ms.com), Sorcerer (www.sagenresearch.com), OMSSA (www.pubchem.ncbi.nlm.nih.gov/omssa/), X!Tandem (www.thegpm.org/TANDEM/), Protein Prospector (prospector.ucsf.edu/prospector/mshome.htm), Byonic (www.proteinmetrics.com/products/byonic) or Sequest (fields.scripps.edu/sequest).

In some embodiments, the method for identifying at least one product-related variant, for determining an effect of at least one PTM or for characterizing a PTM can comprise using a competitive binding assay with insufficient antigen immobilized on a solid surface.

As used herein, the term “solid surface” can include any surface with an ability to bind to a capture molecule, for example an antigen. Non-limiting examples of a solid surface can include affinity resins, beads and coated plates with an immobilized protein, such as, avidin, streptavidin, or NeutrAvidin.

In some embodiments, the sample comprising the protein of interest can be digested after the competitive binding assay but prior to a separation and/or quantitation step, for example SCX-MS.

In some embodiments, the sample comprising the protein of interest can be treated by adding a reducing agent to the sample.

As used herein, the term “reducing” refers to the reduction of disulfide bridges in a protein. Non-limiting examples of the reducing agents used to reduce the protein are dithiothreitol (DTT), 8-mercaptoethanol, Ellman's reagent, hydroxylamine hydrochloride, sodium cyanoborohydride, tris(2-carboxyethyl)phosphine hydrochloride (TCEP-HCl), or combinations thereof. In some specific embodiments, the treatment can further include alkylation. In some other specific exemplary embodiments, the treatment can include alkylation of sulfhydryl groups on a protein.

As used herein, the term “treating” or “isotopically labeling” can refer to chemical labeling a protein. Non-limiting examples of methods to chemically label a protein include Isobaric tags for relative and absolute quantitation (iTRAQ) using reagents, such as 4-plex, 6-plex, and 8-plex; reductive demethylation of amines, carbamylation of amines, ¹⁸O-labeling on the C-terminus of the protein, or any amine- or sulfhydryl-group of the protein to label amines or sulfhydryl group.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion.

As used herein, the term “hydrolyzing agent” refers to any one or combination of a large number of different agents that can perform digestion of a protein. Non-limiting examples of hydrolyzing agents that can carry out enzymatic digestion include trypsin, endoproteinase Arg-C, endoproteinase Asp-N, endoproteinase Glu-C, outer membrane protease T (OmpT), immunoglobulin-degrading enzyme of Streptococcus pyogenes (IdeS), chymotrypsin, pepsin, thermolysin, papain, pronase, and protease from Aspergillus Saitoi. Non-limiting examples of hydrolyzing agents that can carry out non-enzymatic digestion include the use of high temperature, microwave, ultrasound, high pressure, infrared, solvents (non-limiting examples are ethanol and acetonitrile), immobilized enzyme digestion (IMER), magnetic particle immobilized enzymes, and on-chip immobilized enzymes. For a recent review discussing the available techniques for protein digestion see Switazar et al., “Protein Digestion: An Overview of the Available Techniques and Recent Developments” (J. Proteome Research 2013, 12, 1067-1077). One or a combination of hydrolyzing agents can cleave peptide bonds in a protein or polypeptide, in a sequence-specific manner, generating a predictable collection of shorter peptides.

### Exemplary Embodiments

Embodiments disclosed herein provide methods for identifying at least one product-related variant in a sample comprising a protein of interest.

In some exemplary embodiments, this disclosure provides a method for identifying at least one product-related variant in a sample comprising a protein of interest, contacting a sample including a protein of interest and at least one product-related variant to a competitive binding condition, wherein a binding condition provides an insufficient antigen immobilized on beads and wherein said at least one product-related variant has compromised binding with said insufficient antigen; incubating said sample with said insufficient antigen; collecting a flow-through from washing after incubating; and identifying the at least one product-related critical quality attributes in said flow-through using a liquid chromatography-mass spectrometer.

In some exemplary embodiments, a product-related variant is one or more of truncated forms, modified forms, and aggregates of the protein of interest.

In some exemplary embodiments, a product-related variant is deamidated, isomerized, mismatched S—S linked, oxidized, and/or altered conjugated form (e.g., glycosylation, phosphorylation) of the protein of interest.

In some exemplary embodiments, a product-related variant is a post-translationally modified form.

In some exemplary embodiments, a product-related variant has a compromised binding affinity, wherein the compromised binding affinity is about 90% the binding affinity of the protein of interest, about 80% the binding affinity of the protein of interest, about 70% the binding affinity of the protein of interest, about 60% the binding affinity of the protein of interest, about 50% the binding affinity of the protein of interest, about 40% the binding affinity of the protein of interest, about 30% the binding affinity of the protein of interest, about 20% the binding affinity of the protein of interest, or is about 10% the binding affinity of the protein of interest.

In some exemplary embodiments, the mass spectrometer can be a nano-electrospray ionization mass spectrometer.

In some exemplary embodiments, the electrospray ionization mass spectrometer can be run under native conditions.

It is understood that the methods are not limited to any of the aforesaid protein, impurity, and column and that the methods for identifying or quantifying may be conducted by any suitable means.

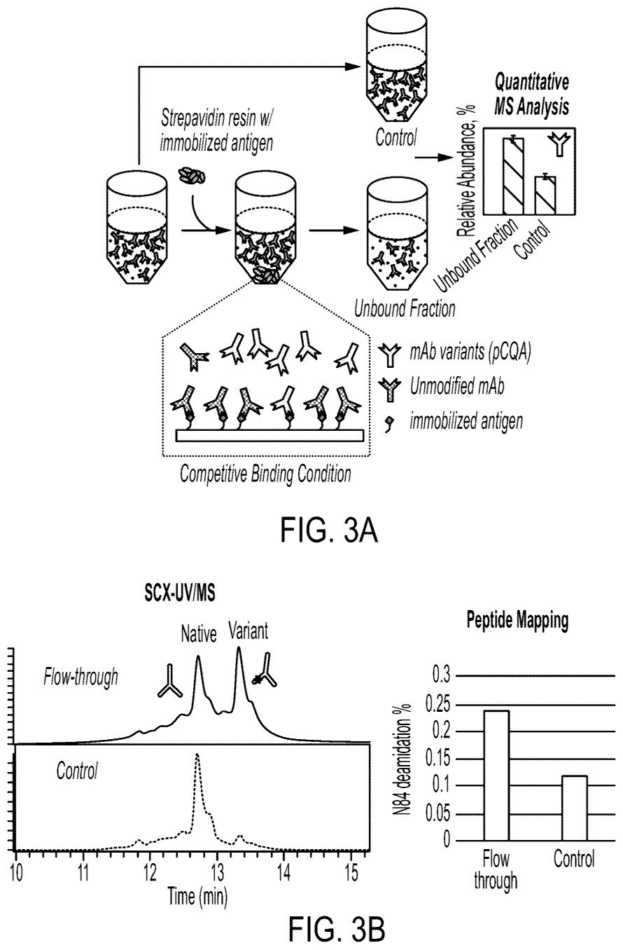

An exemplary embodiment is illustrated in FIGS. 3A and 3B. To a sample comprising the protein if interest and possibly its variants, beads with immobilized antigen can be added. The amount of beads with immobilized antigen is such that not all the protein of interest (native mAb) and its variants can bind to it. Any variant with a reduced binding affinity to the antigen will have lower chance to bind due to the limited amount of antigen present. The flow-through (unbound fraction) can be collected and analyzed using SCX-MS or peptide mapping. The control (i.e., the sample without the immobilized antigen binding assay step) can also be analyzed using SCX-MS or peptide mapping. The comparative study between the flow-through and control can lead to a chromatogram as illustrated in FIG. 3B. Any variant with reduced binding affinity would be more abundant in the flow-through. On comparison of the amount of the variant as such identified, it can be seen that the relative percentage of the variant is more in the flow-through than the control due to its reduced binding affinity.

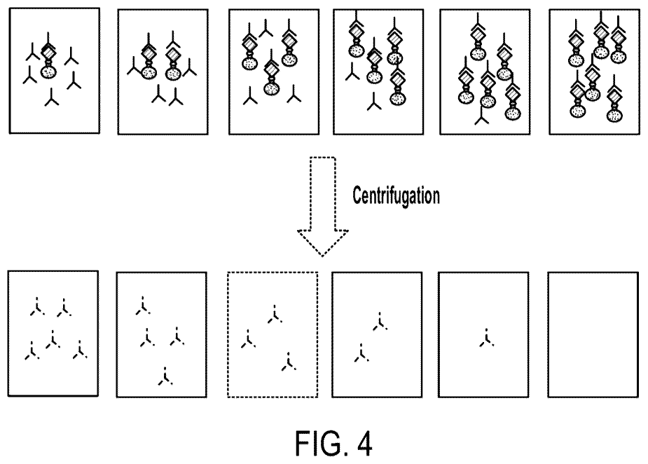

Such an experiment can be devised using the workflow as shown in FIG. 4.

For the present invention, the ratio between the antigen and protein of interest is very important. The amount of the antigen added can be such that about 25% to about 90% of the protein of interest can bind to the antigen. In some embodiments, the amount of the antigen added can be such that about 50% of the protein of interest can bind to the antigen.

The consecutive labeling of method steps as provided herein with numbers and/or letters is not meant to limit the method or any embodiments thereof to the particular indicated order.

Various publications, including patents, patent applications, published patent applications, accession numbers, technical articles and scholarly articles are cited throughout the specification. Each of these cited references is herein incorporated by reference, in its entirety and for all purposes.

The disclosure will be more fully understood by reference to the following Examples, which are provided to describe the disclosure in greater detail. They are intended to illustrate examples and should not be construed as limiting the scope of the disclosure.

### EXAMPLES

Chemicals and reagents. bsAb-1, bsAb-2, msAb-1, msAb-2, and their corresponding antigens were all produced at Regeneron (Tarrytown, NY). Ammonium acetate (MS grade), acetic acid (MS grade), urea, and Amicon centrifugal filters were obtained from Sigma-Aldrich (St. Louis, MO). EZ-Link™ Sulfo-NHS-LC-LC-Biotin, Pierce™ streptavidin agarose resin, Pierce™ micro-spin columns, acetonitrile (ACN; LC-MS grade), formic acid (FA), dithiothreitol (DTT), iodoacetamide (IAA), and Invitrogen UltraPure 1 M Tris-HCl buffer, pH 7.5 were purchased from Thermo Fisher Scientific (Waltham, MA). Sequencing grade modified trypsin was purchased from Promega (Madison, WI). A BioPro IEX SF column (4.6 mm×100 mm, 5 μm; YMC Co., Ltd., Kyoto, Japan) and BioPro QA-F SAX column (4.6 mm×100 mm, 5 μm; YMC Co., Ltd., Kyoto, Japan) were used for SCX and AEX separation, respectively. A C18 column (ACQUITY UPLC peptide BEH 1.7 μm, 2.1 mm×150 mm, Waters) was used for peptide mapping analysis.

Antigen biotinylation and conjugation with streptavidin resin. All antigens were biotinylated using EZ-Link™ Sulfo-NHS-LC-LC-Biotin. Immediately before use, a 10 mM biotin solution was prepared by dissolving 2.0 mg reagent in 300 μL of water. Antigens were incubated at concentrations between 2 to 5 mg/mL in the presence of 12-fold molar excess of biotin at room temperature for 30 minutes. After biotinylation, excess biotin reagent was removed using an Amicon centrifugal filter unit (10 kDa MW cut-off) and buffer exchanged into 100 mM Tris-HCl (pH 7.5). The concentration of the biotinylated antigen was then determined using Nanodrop 2000 spectrophotometer (ThermoFisher Scientific, Waltham, MA USA) by measuring UV absorbance at 280 nm. Biotinylated antigen was conjugated onto streptavidin agarose resin by incubating biotinylated antigen and settled resin at 2×10⁻⁵ μmole antigen per μL of resin for one hour at room temperature. The conjugated resin was then washed and equilibrated using 100 mM Tris-HCl buffer, pH 7.5.

Competitive binding experiment. To titrate the competitive binding conditions, an increasing amount of antigen-immobilized resin (2 μL, 4 μL, 8 μL, 10 μL, 20 μL, 40 μL, 60 μL, 80 μL, 100 μL, 120 μL, 140 μL, and 160 μL) was placed into a micro-spin column and the solution was removed by spinning down at 3000 g for 1 min. 10-20 uL of mAb solution containing ˜0.4 nmole of mAb was then added into each micro-spin column and incubated at room temperature for 45 minutes. The mixture was then spun down at 3000 g for 1 minute to collect the flow-through fraction. Both the mAb control sample and the flow-through fraction were then subjected to UV absorbance measurement at 280 nm to calculate the relative percentage of the mAb in the flow-through fraction (i.e., the depletion level). For the competitive binding experiment, mAb sample was incubated with a selected amount of the antigen-immobilized resin, which was pre-determined from the titration experiment, at room temperature for 45 minutes. The flow-through fraction was then collected by centrifugation at 3000 g for 1 minute. Flow-through fraction and the unfractionated control sample were both subjected to intact (top-down) MS analysis or peptide mapping (bottom-up) analysis.

Intact MS analysis using SCX-MS and AEX-MS. For SCX-MS analysis, a BioPro IEX SF column (4.6 mm×100 mm, 5 μm; YMC Co., Ltd., Kyoto, Japan) was used at 45° C. with a linear gradient of 20 mM ammonium acetate (pH 5.6, adjusted with acetic acid) to 150 mM ammonium acetate (pH 6.8) for 16 minutes at 0.4 mL/min. For AEX-MS analysis, a BioPro QA-F SAX column (4.6 mm×100 mm, 5 μm; YMC Co., Ltd., Kyoto, Japan) was used at 45° C. with a linear gradient of 10 mM ammonium acetate (pH 6.8) to 300 mM ammonium acetate (pH 6.8) for 16 minutes at 0.4 mL/min. An aliquot of 5.0 μL of protein was loaded on the column. A Thermo Q Exactive UHMR mass spectrometer equipped with a Newomics MnESI ionization source and a Microfabricated Monolithic Multi-nozzle (M3) emitter (Berkeley, CA) was used for data acquisition. The detailed mass spectrometry settings can be found in a previous publication (Yan et al., 2020, Journal of the American Society for Mass Spectrometry, 31:2171-9).

Tryptic digestion and peptide mapping analysis. Each flow-through fraction and a corresponding unfractionated control sample (20 μL) was mixed with 100 μL of 8 M urea and 10 mM DTT in 0.1 M Tris-HCl (pH 7.5) and incubated at 50° C. for 30 minutes. The denatured and reduced samples were then alkylated with 25 mM IAA at room temperature in the dark for 30 minutes, followed by dilution with 0.1 M Tris-HCl (pH 7.5) to 600 μL. Each sample was treated with 20 μg trypsin and digested at 37° C. overnight. Digestion was halted by adding formic acid (FA) to 1%. Approximately 1.2 μg (for msAb) or 6 μg (for bsAb) of the digested sample was loaded onto a C18 column (ACQUITY UPLC peptide BEH 1.7 μm, 2.1 mm×150 mm, Waters) and separated by a 90 min gradient with 0.1% FA in water as mobile phase A, and 0.1% FA in acetonitrile as mobile phase B (0-5 min, 0.1% B; 5-80 min, 0.1-35% B; 80-85 min, 35-90% B; 85-90 min, 90% B). The mobile phase flow rate was 0.25 mL/min. The column temperature was set at 40° C. The instrument source parameters were set as follows: spray voltage 3.8 kV, auxiliary gas 10, auxiliary gas temperature 250° C., capillary temperature 350° C., and S-lens RF level 50. A top five data-dependent acquisition method (DDA) was applied for MS/MS data acquisition during online LC separation. The following settings were applied for MS1 scans: resolution 70k, AGC target 1×106, maximum ion injection time 50 ms, and scan range 300-2000 m/z. For HCD MS/MS scans, the following settings were applied: isolation window 4 m/z, NCE 27, scan range 200-2000 m/z, 17.5k resolution, AGC target 1×105, and maximum ion injection time 100 ms. The raw data files were searched against the antibody FASTA sequences by Byonic (version 3.9.4, Protein Metrics, San Carlos, CA) for post-translational modification identification.

Data analysis. All experiments were performed in triplicate. Integration of the XIC peak area for the SCX-MS and AEX-MS analyses was performed using Thermo Fisher Xcalibur software (version 3.0). Peptide and PTM identification were achieved using Byonic software (version 3.9.4, Protein Metrics, San Carlos, CA). PTM quantitation at peptide level was performed using Skyline software (version 20.1, MacCoss Lab Software, Seattle, USA). The t-test was performed using GraphPad Prism (version 8.0, GraphPad Software Inc., San Diego, CA).

Biolayer interferometry (BLI). An Octet® BLI platform was used. Anti-human IgG Fc antibody coated biosensor tips were used. msAb-3 was incubated in the wells to bind to the anti-Fc antibodies. msAb-3 target antigen was added and binding was measured.

Cell-based bioassay. HEK293 cells with a luciferase gene linked to the STAT5 gene were used. 20,000 cells were seeded per 80 uL of media. Cells were incubated overnight at 37 C and 5% CO2. 20 uL of msAb-3 was added (200 nM 1:3). 20 uL of ligand was added (3 nM). The cells were incubated for 5 hours at 37 C and 5% CO2. 120 uL of ONE-Glo™ luciferase substrate was added. Luciferase signal was analyzed using an EnVision plate reader.

### Example 1. Competitive Binding MS Workflow

In order to develop a simple and high-throughput method for characterizing differences in target binding affinity due to the presence of various protein attributes, a competitive binding environment was designed to enable the enrichment of protein variants with decreased target binding affinity. An exemplary workflow is illustrated in FIG. 3A. First, a sample including a protein of interest, for example a mAb sample, is incubated with an insufficient amount of an immobilized capture molecule, for example an antigen, so that the total amount of the antibody exceeds the binding capacity of the antigen. As a result, the unmodified mAb species will be preferentially bound to the immobilized antigen, while mAb variants with decreased binding affinity will be enriched in the unbound fraction. Subsequently, quantitative mass spectrometry analysis either at intact mAb level or after tryptic digestion can be applied to compare the relative abundances of each attribute in the unbound fraction and in the unfractionated control sample. Finally, attributes that show significant enrichment in the unbound fraction indicate compromised target binding affinity, and thus should be considered as potential CQAs.

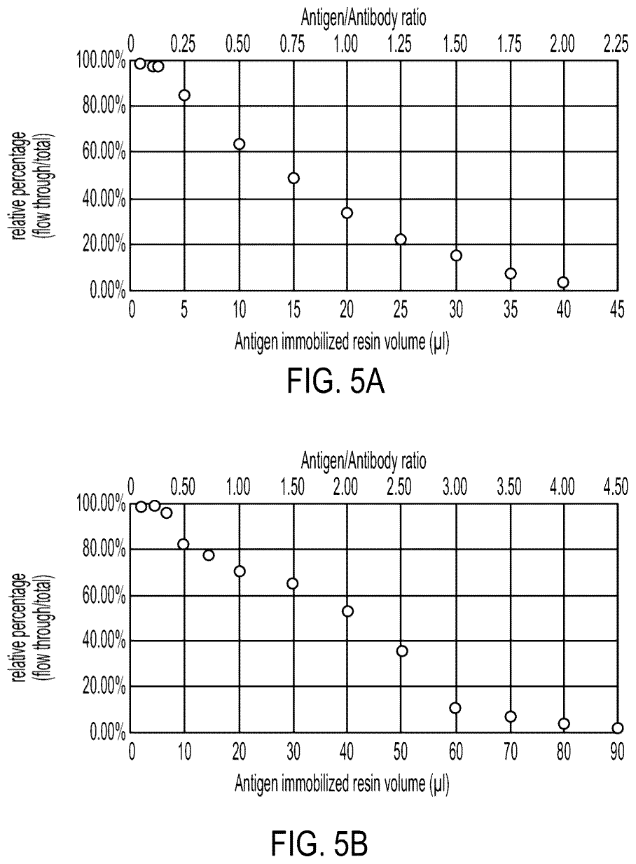

To ensure a competitive binding environment, the molar ratio between the immobilized antigen and the applied mAb needs to be well controlled and characterized. Although the protein concentrations of both the antigen and the mAb stock solutions are easily accessible, the antigen biotinylation and subsequent immobilization processes can introduce variables that affect the actual antigen-to-mAb ratio. Therefore, to accurately determine the mixing ratio, a titration experiment was performed by mixing an increasing amount of immobilized antigen to a fixed amount of mAb sample. Increasing amounts of immobilized antigen were added to a fixed amount of specific antibody. After incubation, unbound fractions were collected in the flow-through by centrifugation. The ratio of the unbound antibody to the total antibody added for incubation was calculated by measuring the antibody concentration in the flow-through using UV absorbance at 280 nm. After normalizing to the total mAb amount, the relative abundance of the unbound mAb in each sample was calculated and plotted against the volume of the antigen resin used. By plotting the relative percentage of unbound antibody versus the amount of resin used for incubation, an amount of antigen that should be used for a competitive binding experiment can be determined, as shown in FIG. 5A for bispecific antibody and FIG. 5B for monospecific antibody.

As expected, a decreasing amount of the unbound mAb was observed as the amount of antigen was increased, until the unbound mAb was completely depleted. In theory, the entire region of this titration curve till the complete depletion of the unbound mAb can be considered competitive binding conditions. As more mAb species (e.g., mostly unmodified mAb) are depleted from the unbound fraction, a greater enrichment of pCQA-related mAb variants is expected, therefore facilitating their identification.

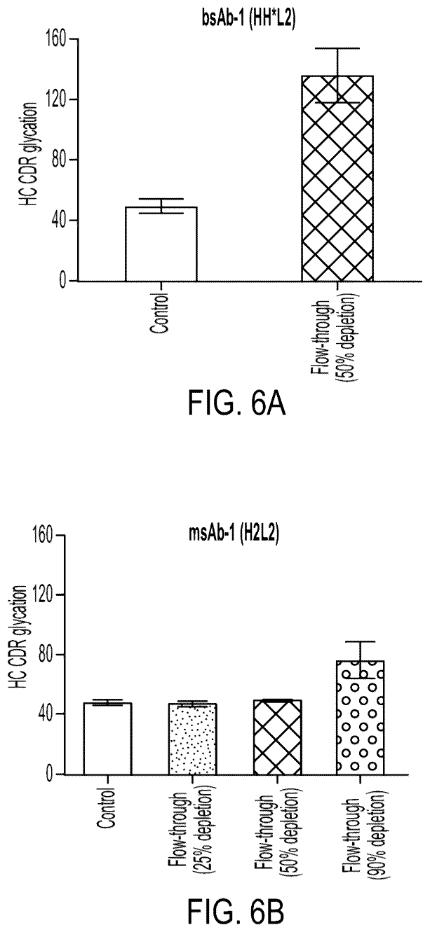

The extent of mAb depletion required for successful pCQA determination can be different for bispecific antibodies (bsAbs) and monospecific antibodies (msAbs). For a bsAb molecule, a critical CDR modification from its unique Fab arm might dramatically affect its binding ability with the corresponding antigen. In contrast, the same CDR modification occurring on one of the two identical Fab arms in a msAb molecule might only negligibly impact its binding ability with the antigen, due to the availability of one unmodified Fab arm. To understand the extent of mAb depletion required for successful pCQA determination, a bsAb (HH*L2, bsAb-1) and its homodimer (H2L2, msAb-1), both containing the same critical modification (a CDR Lys glycation known to reduce antigen binding affinity) in the heavy chain (H) CDR region, were tested under various competitive binding conditions using nSCX-MS. For the bsAb, depletion of only half of the total antibody by the immobilized antigen was sufficient to result in a significant enrichment of this CDR Lys glycation in the unbound fraction, demonstrating its critical impact on target binding, as shown in FIG. 6A. In contrast, for the msAb, the same extent of antibody depletion (i.e., 50%) did not lead to any enrichment of this modification in the unbound fraction. Instead, a significant enrichment of this modification was only observed after 90% of the total antibody was depleted from the unbound fraction, as shown in FIG. 6B. Therefore, to ensure effective enrichment of mAb variants containing pCQAs, depletion levels of 50% and 90% were selected for bsAb and msAb, respectively, to perform the competitive binding MS experiments.

### Example 2. Evaluation of Potential CQAs by Competitive Binding and IEX-MS Analysis

After fractionation of the mAb variants under competitive binding conditions, quantitative MS analysis may be performed to examine whether the attributes-of-interest are significantly enriched in the unbound fraction. Ion exchange chromatography coupled with native MS is a powerful technique to achieve rapid quantitation of a wide range of CDR modifications, due to its excellent selectivity towards surface modifications. To demonstrate the validity of this approach, three mAb examples, each containing a specific CDR modification at a notable level, were subjected to competitive binding followed by either native SCX-MS (strong cation exchange chromatography coupled to native MS) or native AEX-MS (anion exchange chromatography coupled to native MS) analysis.

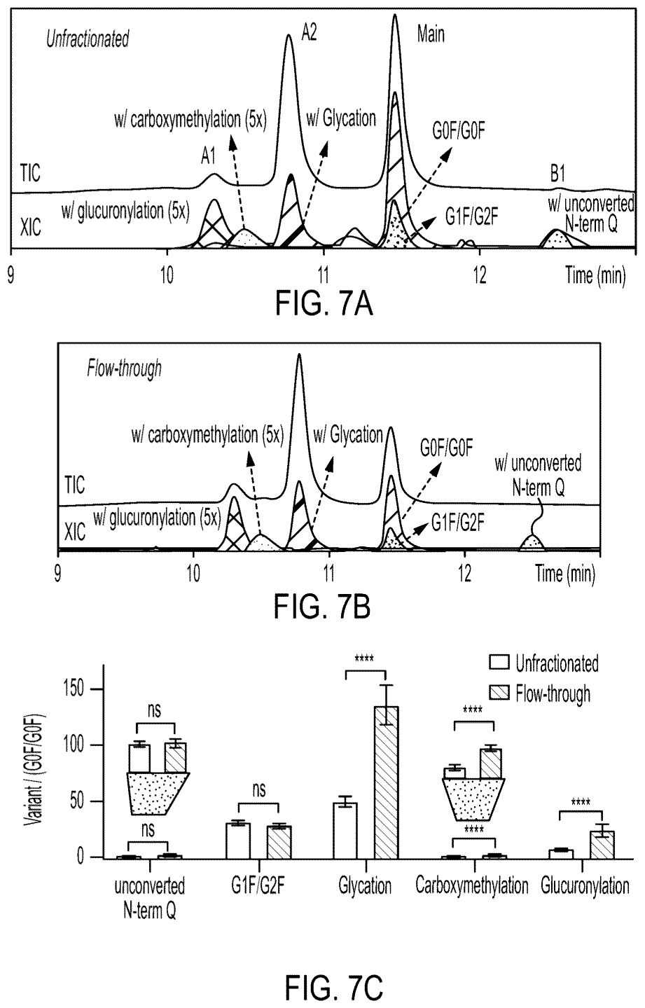

A first mAb molecule characterized using the method of the present invention was the bispecific antibody bsAb-1. It consists of two identical light chains (LC) and two different heavy chains (HC and HC*). This molecule contains a high level of glycation on the Lys98 residue within the HC CDR3 region, which is known to reduce the binding affinity to its corresponding target based on surface plasmon resonance (SPR)-based measurement. After competitive binding using the HC-corresponding antigen, the unbound fraction and the unfractionated control samples were both subjected to native SCX-MS analysis, which was previously shown to separate this CDR Lys98 glycation variant (red, FIG. 7A and FIG. 7B) as a defined acidic peak. In addition, this method can also monitor a CDR glucuronylation variant (orange, FIG. 7A and FIG. 7B) and a CDR carboxymethylation variant (magenta, FIG. 7A and FIG. 7B) occurring on the same Lys98 residue. Other mAb variants, resulting from HC N-terminal heterogeneity (e.g., non-cyclized Gln, cyan, FIG. 7A and FIG. 7B) and Fc N-glycosylation microheterogeneity (e.g., galactosylation, green, FIG. 7A and FIG. 7B) can also be monitored using the extracted ion chromatograms (XICs), as shown in FIG. 7A and FIG. 7B. Subsequently, the relative abundance of each attribute can be calculated using its XIC peak area normalized to that of the main species (G0F/G0F, blue, FIG. 7A and FIG. 7B) and compared between the unfractionated control sample and the unbound fraction (FIG. 7C).

It is clear using the method of the present invention that all three modifications (glycation, glucuronylation, and carboxymethylation) occurring on HC CDR Lys98 were significantly enriched in the unbound fraction under competitive binding conditions, indicating the compromised target binding of these variants. In contrast, other attributes, including the non-cyclized N-terminal Gln and the galactosylation of the Fc N-glycan, showed no quantitative difference between the two samples, consistent with the common knowledge that they do not contribute to target binding (FIG. 7C). It is worth noting that although the criticality of Lys98 glycation has already been confirmed by SPR-based binding analysis of the enriched material (i.e., through SCX fractionation), the impact of Lys98 glucuronylation and carboxymethylation had remained unknown due to the difficulty in enriching them to sufficient quantities. Therefore, this competitive binding and IEX-MS workflow is valuable to provide orthogonal evaluation of the CDR modifications that might be difficult to study using traditional approaches.

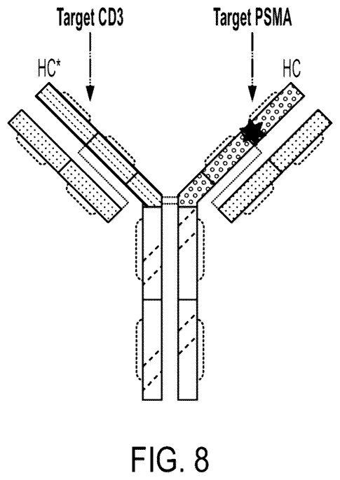

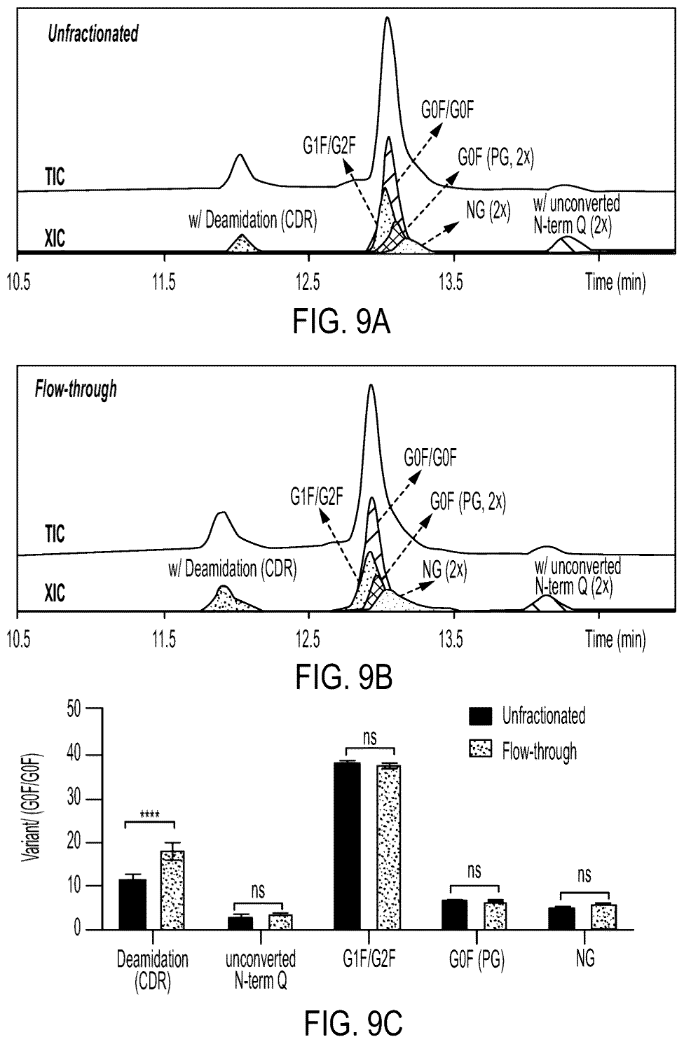

In a further example, a bsAb (bsAb-2) containing a notable level of deamidation on the HC CDR3 Asn56 residue was studied by competitive binding using its corresponding antigen followed by SCX-MS analysis. As this site-specific CDR deamidation can be well separated by the SCX-MS method, quantitation of this deamidation can be readily achieved at intact mAb level without performing peptide mapping. The structure of bsAb-2 is illustrated in FIG. 8. By evaluating the XIC, the elution profile of the Asn56 deamidation variant can be illustrated (red, FIG. 9A and FIG. 9B) and its abundance relative to the main species (G0F/G0F, blue, FIG. 9A and FIG. 9B) can be calculated in each of the unfractionated control sample and the unbound fraction. Quantitative comparison from triplicate measurements indicated a significant enrichment of this CDR PTM from the unfractionated control sample to the unbound fraction (FIG. 9C), which suggested that this CDR deamidation negatively impacts target binding of bsAb-2. As negative controls, other attributes that are not expected to affect target binding (e.g., the non-cyclized N-terminal Gln and Fc N-glycosylation macro- and micro-heterogeneity) showed highly comparable relative abundances between the two samples. Therefore, it was concluded that HC CDR Asn56 deamidation in bsAb-2 should be considered as a potential CQA that needs to be further studied and closely monitored during its development.

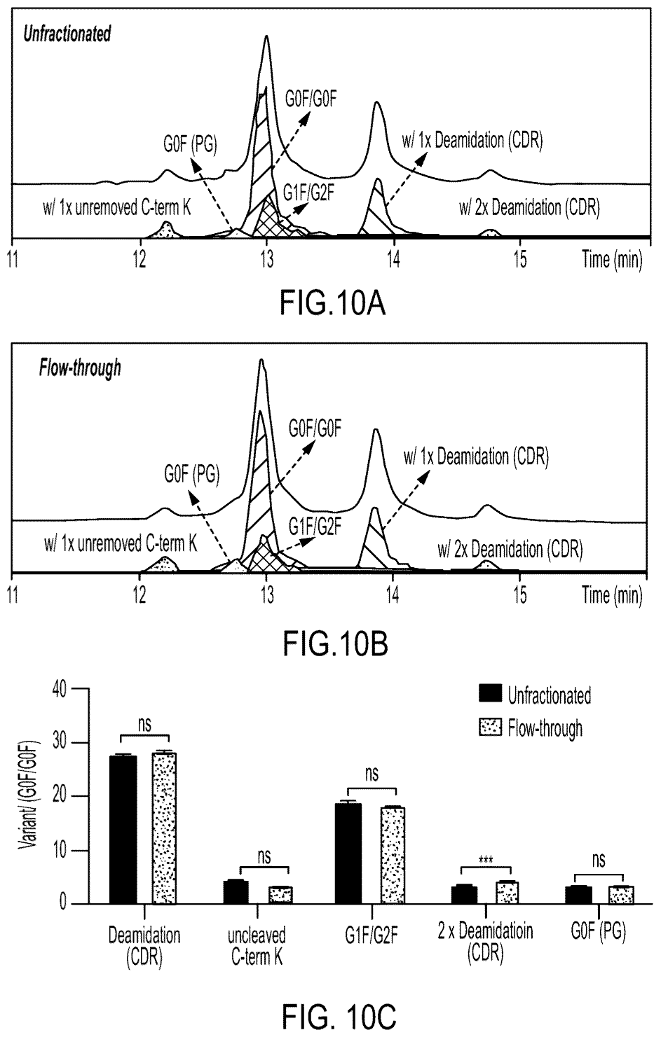

In an additional example, another CDR deamidation (HC Asn56) occurring in a msAb (msAb-2) was also evaluated by competitive binding and IEX-MS workflow. As discussed earlier, to effectively enrich variants in a msAb, a higher antigen-to-antibody ratio was required to achieve a greater depletion of unbound mAb species. In this example, a competitive binding condition was applied so that only 10% of the mAb species were isolated into the unbound fraction for subsequent MS analysis. Furthermore, as msAb-2 is an IgG4 molecule with a relatively low isoelectric point (pI), an AEX-MS method was found to provide an improved charge variant separation compared to the more commonly applied SCX-MS method. As shown in FIG. 10A and FIG. 10B, msAb-2 variants with Asn56 deamidated on one of its two heavy chains were readily separated as an acidic peak during AEX-MS analysis. In addition, a low level of variants with both HC Asn56 deamidated was also observed as a far acidic peak. After competitive binding experiments, AEX-MS analysis of the unfractionated control sample and the unbound fraction showed a highly comparable charge variant profile. Quantitative analysis using XIC-based approach also demonstrated that, similar to other attributes that are not expected to impact antigen binding (e.g., C-terminal Lys variant and Fc N-glycosylation heterogeneity), variants with just one Asn56 deamidated were not significantly enriched in the unbound fraction. Interestingly, variants with both Asn56 deamidated did show a very minor, but statistically significant enrichment in the unbound fraction (FIG. 10C). Therefore, it was concluded that although Asn56 deamidation occurs within a CDR region, its impact on antigen binding might be limited. Although a complete evaluation of this CDR deamidation still requires further studies, the competitive binding and IEX-MS workflow provided an early readout of its impact, which is particularly valuable for candidate developability assessment.

### Example 3. Evaluation of Potential CQAs by Competitive Binding and Bottom-Up Analysis

Although IEX-MS provides a rapid means of quantifying several attributes in mAb samples with very limited sample processing, it cannot quantify many other attributes that are not resolved by either mass or LC retention time. In particular, site-specific modifications that are not resolved by IEX separation cannot be reliably quantified by intact mass approaches. In this case, competitive binding followed by bottom-up analysis can be applied to achieve a more comprehensive assessment of attributes. In addition, as more common attributes (i.e., the ones not expected to affect target binding) can be quantified in this workflow, more negative controls can be included to facilitate reliable determination of pCQAs.

Briefly, after competitive binding experiments, the unfractionated control sample and the unbound fraction were both subjected to trypsin digestion followed by LC-MS/MS analysis. After identifying each attribute (i.e., modification) at tryptic peptide level, its relative abundance was calculated using the integrated XIC peak areas from both the modified and the unmodified peptides, and subsequently compared between the two samples.

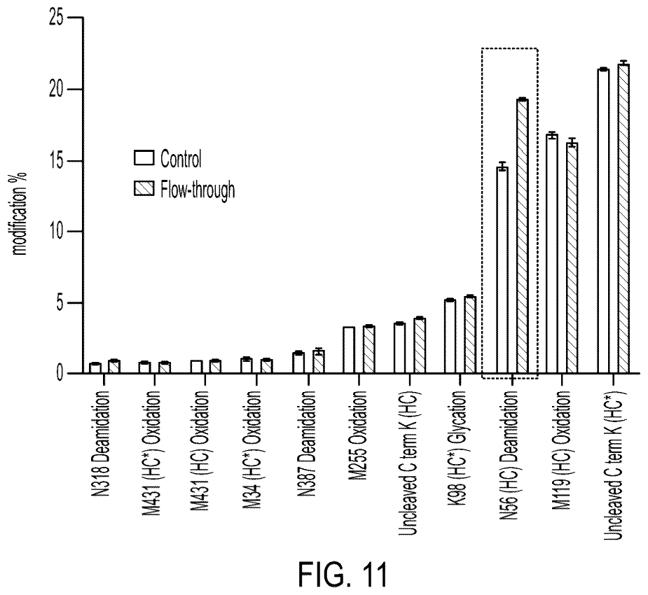

To demonstrate the utility of this workflow, the three mAb molecules that were previously studied by IEX-MS were also subjected to tryptic digestion-based bottom-up analysis. Each attribute for each mAb was quantified, as shown in Tables 1-3 and for bsAb-2 in FIG. 11.

TABLE 1
PTM quantitation results by peptide mapping for flow-through sample and unfractionated sample for bsAb-1

| | Control | | | Flow-through | | |
|---|---|---|---|---|---|---|
| PTM | Replicate 1 | Replicate 2 | Replicate 3 | Replicate 1 | Replicate 2 | Replicate 3 |
| M4 Oxidation (LC) | 0.93% | 1.04% | 1.10% | 1.18% | 1.12% | 1.10% |
| N138 Deamidation (LC) | 0.19% | 0.21% | 0.18% | 0.16% | 0.17% | 0.20% |
| M248 (HC)/M255 (HC*) | 3.63% | 3.72% | 3.85% | 3.98% | 4.47% | 4.01% |
| Oxidation | | | | | | |
| N380 (HC)/N387 (HC*) | 1.17% | 1.29% | 1.24% | 1.02% | 1.15% | 1.15% |
| Deamidation | | | | | | |
| N385 (HC)/N392 (HC*) | 1.04% | 1.03% | 1.06% | 0.92% | 0.98% | 0.99% |
| Deamidation | | | | | | |
| M34 (HC*) Oxidation | 4.45% | 5.14% | 5.22% | 5.85% | 5.66% | 6.08% |
| M83 (HC*) Oxidation | 1.90% | 1.92% | 1.99% | 2.01% | 2.11% | 2.20% |
| N311 (HC)/N318 (HC*) | 0.27% | 0.29% | 0.27% | 0.27% | 0.28% | 0.28% |
| Deamidation | | | | | | |
| M341 (HC*) Oxidation | 2.60% | 2.64% | 2.79% | 2.92% | 2.16% | 2.16% |
| K98 (HC) Glycation | 37.37% | 38.17% | 37.86% | 53.33% | 53.87% | 55.06% |
| K98 (HC) CML | 1.43% | 1.39% | 1.39% | 2.08% | 2.12% | 2.05% |
| K98 (HC) Glucuronylation | 1.84% | 1.82% | 1.83% | 2.78% | 2.74% | 2.68% |
| M34 (HC) Oxidation | 4.16% | 4.21% | 4.12% | 4.81% | 4.53% | 4.11% |
| N84 (HC) Deamidation | 0.70% | 0.68% | 0.68% | 0.73% | 0.69% | 0.68% |
| Unprocessed N term Q | 2.10% | 2.34% | 2.29% | 2.34% | 2.24% | 2.43% |
| Uncleaved C term K (HC) | 1.70% | 1.54% | 1.83% | 1.69% | 1.85% | 1.90% |
| Uncleaved C term K (HC*) | 16.21% | 15.84% | 17.32% | 18.21% | 16.12% | 16.17% |

TABLE 2
PTM quantitation results by peptide mapping for flow-through sample and unfractionated sample for bsAb-2

| | Control | | | Flow-through | | |
|---|---|---|---|---|---|---|
| PTM | Replicate 1 | Replicate 2 | Replicate 3 | Replicate 1 | Replicate 2 | Replicate 3 |
| N364 Deamidation (HC/HC*) | 0.21% | 0.22% | 0.22% | 0.24% | 0.25% | 0.24% |
| N138 Deamidation (LC) | 0.24% | 0.25% | 0.24% | 0.26% | 0.26% | 0.27% |
| N318 Deamidation (HC/HC*) | 0.70% | 0.77% | 0.70% | 0.91% | 0.90% | 0.86% |
| M431 (HC*) Oxidation | 0.83% | 0.82% | 0.83% | 0.83% | 0.85% | 0.77% |
| M431 (HC) Oxidation | 0.92% | 0.92% | 0.92% | 0.93% | 0.96% | 0.88% |
| M34 (HC*) Oxidation | 0.95% | 1.04% | 0.95% | 0.95% | 0.96% | 0.97% |
| N387 Deamidation (HC/HC*) | 1.42% | 1.60% | 1.40% | 1.48% | 1.38% | 1.79% |
| M255 Oxidation (HC/HC*) | 3.32% | 3.32% | 3.32% | 3.28% | 3.37% | 3.34% |
| Uncleaved C term K (HC) | 3.60% | 3.60% | 3.61% | 3.89% | 3.92% | 3.72% |
| K98 (HC*) Glycation | 5.22% | 5.14% | 5.20% | 5.33% | 5.43% | 5.44% |
| N56 (HC) Deamidation | 14.34% | 14.47% | 14.91% | 19.21% | 19.43% | 19.06% |
| M119 (HC) Oxidation | 16.54% | 16.91% | 16.76% | 16.27% | 16.56% | 16.05% |
| Uncleaved C term K (HC*) | 21.30% | 21.59% | 21.32% | 21.74% | 21.98% | 21.64% |
| Unprocessed N term Q (HC/HC*) | 2.41% | 2.43% | 2.46% | 2.51% | 2.48% | 2.42% |
| N328 Deamidation (HC/HC*) | 0.28% | 0.25% | 0.27% | 0.24% | 0.25% | 0.26% |
| M361 Oxidation (HC/HC*) | 0.78% | 0.74% | 0.76% | 0.81% | 0.82% | 0.79% |
| N392 Deamidation (HC/HC*) | 1.70% | 1.82% | 1.90% | 1.86% | 1.93% | 1.95% |
| M83 (HC*) Oxidation | 0.13% | 0.14% | 0.15% | 0.13% | 0.15% | 0.15% |

TABLE 3
PTM quantitation results by peptide mapping for flow-
through sample and unfractionated sample for mAb-1

| | Control | | | Flow through | | |
|---|---|---|---|---|---|---|
| PTM | Replicate 1 | Replicate 2 | Replicate 3 | Replicate 1 | Replicate 2 | Replicate 3 |
| Unprocessed N term Q | 1.11% | 1.13% | 1.14% | 1.13% | 1.10% | 1.11% |
| HC N56 Deamidation | 20.36% | 21.87% | 22.32% | 26.34% | 26.89% | 28.47% |
| HC N77 Deamidation | 0.43% | 0.45% | 0.48% | 0.50% | 0.52% | 0.58% |
| HC M113 Oxidation | 9.50% | 8.67% | 9.33% | 9.59% | 8.56% | 10.98% |
| HC M250 Oxidation | 9.44% | 9.28% | 9.19% | 9.51% | 8.53% | 9.77% |
| G2F | 11.36% | 11.44% | 11.21% | 11.54% | 11.39% | 11.38% |
| G0 | 4.36% | 4.45% | 4.48% | 4.4% | 4.27% | 4.39% |
| G1F | 22.90% | 22.67% | 23.08% | 23.24% | 23.29% | 23.46% |
| G0F | 61.29% | 61.44% | 61.23% | 61.42% | 60.95% | 61.07% |
| HC W311 Oxidation | 0.07% | 0.06% | 0.06% | 0.06% | 0.07% | 0.08% |
| HC N313 Deamidation | 1.79% | 1.78% | 1.77% | 1.69% | 1.81% | 1.86% |
| HC M356 Oxidation | 2.85% | 5.52% | 5.50% | 3.67% | 3.78% | 4.08% |
| HC N359 Deamidation | 0.29% | 0.32% | 0.33% | 0.35% | 0.39% | 0.41% |
| HC D397 Dehydration | 1.36% | 1.13% | 1.21% | 1.14% | 1.02% | 0.99% |
| Uncleaved C term K | 7.88% | 7.82% | 7.56% | 7.62% | 7.32% | 7.26% |
| LC M4 Oxidation | 4.14% | 4.33% | 4.39% | 3.32% | 3.28% | 3.83% |
| LC W35 Oxidation | 0.10% | 0.09% | 0.10% | 0.08% | 0.10% | 0.11% |
| LC K45 Glycation | 9.92% | 10.25% | 8.78% | 8.88% | 8.64% | 8.79% |

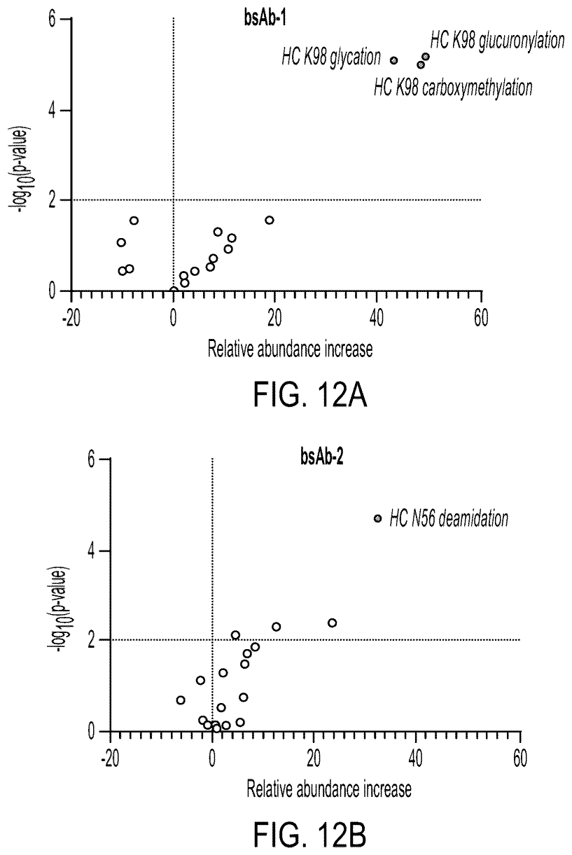

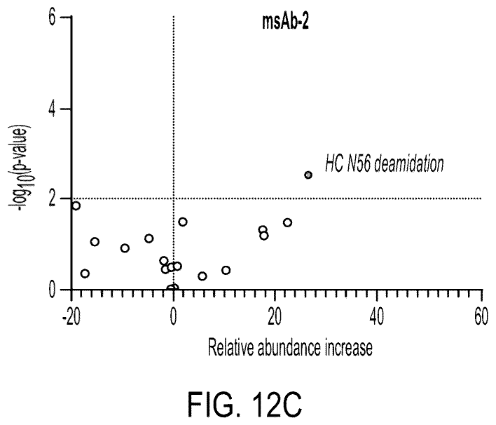

After quantifying each attribute, the percent increase in relative abundance of each attribute (from the unfractionated control sample to the unbound fraction) was plotted against the p value from the Student's t-test based on the three-replicate measurement, as shown in FIG. 12. For bsAb-1, all three HC CDR Lys98 modifications were enriched in the unbound fraction with statistical significance (red dots, FIG. 12A). In contrast, the other 14 attributes showed either an increase or decrease in relative abundance, where both the magnitude of change and the significance were much smaller compared to that of the three CDR modifications (black dots, FIG. 12A). Similarly for bsAb-2, the HC CDR Asn56 deamidation exhibited a significant enrichment in the unbound fraction (red dot, FIG. 12B). Again, other attributes, including modifications on the HC constant domains (deamidation at Asn318 and Asn387, oxidation at Met431 and Met255, and unprocessed HC C-terminal Lys) and modifications on the HC*(oxidation at Met34 and glycation at Lys98) showed no significant quantitative difference between the two samples (black dots, FIG. 12B). Interestingly, Met119 oxidation on the HC variable domain did not seem to impact the HC-corresponding target binding, even though it occurs near the HC CDR3 region. Finally, for msAb-2, the HC CDR Asn56 deamidation was also found to be slightly enriched in the unbound fraction with statistical significance, as shown in FIG. 12C. It is worth noting, though, the quantitation of Asn56 deamidation by bottom-up approach was contributed by both mAb variants with either one or two HC Asn56 deamidated. Although the singly deamidated species were not significantly enriched in the unbound fraction based on the AEX-MS analysis at intact mAb level, the doubly deamidated species were significantly enriched, as shown in FIG. 10C, consistent with the results from the bottom-up analysis. Nevertheless, comparing to the CDR modifications in the other two examples, the HC CDR Asn56 deamidation in msAb-2 did not show a clear separation from the other attributes in both the extent of enrichment and the significance. This suggested that this CDR modification might only impact target binding in a limited way. Comparing to the IEX-MS analysis at intact mAb level, the bottom-up approach can simultaneously quantify many PTMs present at very low abundances (e.g., 0.1%), and thus achieve high-throughput evaluation of multiple attributes.

During the development of therapeutic mAbs, identification of pCQAs is an important step that provides guidance during candidate selection and a framework for risk assessment. Traditionally, identification of target binding-associated pCQA involves a laborious and low-throughput variant enrichment step prior to binding affinity measurement. This disclosure sets forth a novel competitive binding-MS strategy that enables high-throughput and multiplexed assessment of pCQAs directly from unfractionated and unstressed mAb drug samples. Unlike reported methods, the developed workflow performs competitive binding on the immobilized antigen, thus allowing the enrichment of mAb variants with impaired target binding in the unbound fraction. Following the enrichment, quantitative mass spectrometry approaches were adopted to compare the relative abundances of multiple attributes in the control (unfractionated) sample and in the unbound fraction, leading to pCQA identification. To ensure a proper competitive binding environment, a titration experiment was performed to determine the mixing ratio of the immobilized antigen to antibody. Using a bsAb (HH*L2) and its homodimer (H2L2) as model systems, it was determined that the desired extent of antibody depletion is moderate for bsAb analysis (e.g., 50%) but much more stringent for msAb analysis (>90%). The validity and utility of this method was demonstrated in three mAb case studies, including two bsAb molecules and one msAb molecule, where different CDR and non-CDR attributes were successfully interrogated for their impact on antigen binding. It was also shown that a native IEX-MS method can be applied to achieve rapid quantitation of multiple CDR modifications at the intact mAb level. On the other hand, bottom-up analysis can provide a more comprehensive assessment of many attributes, thanks to its excellent resolving power and sensitivity. Using common attributes as negative controls, the identification of pCQAs can be reliably achieved.

In summary, this newly developed competitive binding-MS approach offers several unique advantages over conventional strategies in pCQA identification, including higher throughput, greater sensitivity, and broader applicability. This new method is particularly desirable during the early-stage drug developability assessment, where limited sample material is available and fast turn-around time is often required. Lastly, in addition to target binding, other attributes that are critical for various Fc receptor binding can also be evaluated using the same platform. For example, using neonatal Fc receptor (FcRn) as the binding target, attributes that impact FcRn-antibody binding can be evaluated, which are important for the half-life of the drug molecule (Andersen et al., 2014, Journal of Biological Chemistry, 289:13492-502; Stracke et al., 2014, MAbs, 6(5):1229-42). Similarly, using FcTR as binding target, attributes associated with Fc-mediated effector functions, such as antibody dependent cell-mediated cytotoxicity (ADCC) or complement dependent cytotoxicity (CDC), can also be evaluated (Iida et al., 2006, Clinical Cancer Research, 12:2879-87; Niwa et al., 2004, Clinical Cancer Research, 10:6248-55).

### Example 4. Affinity Fractionation Method for pCQA Identification

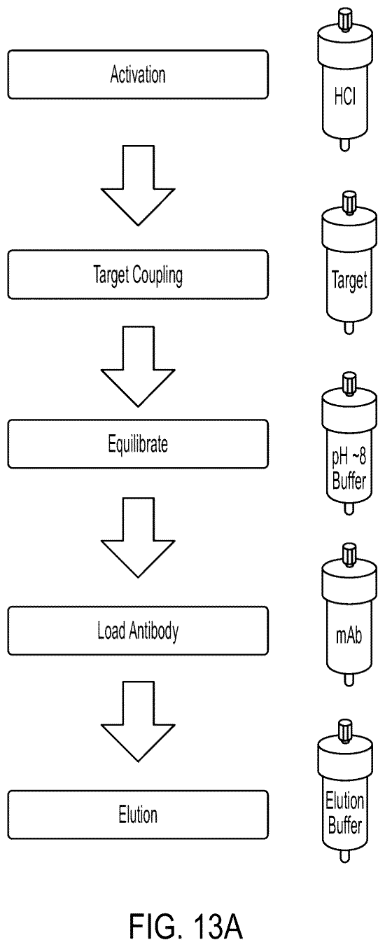

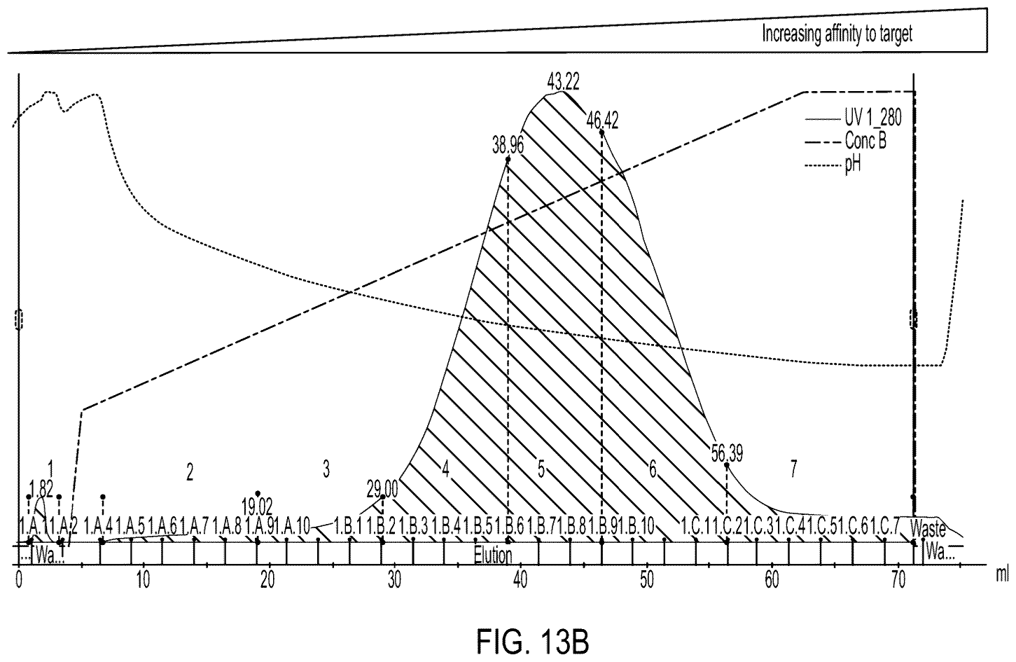

Additional methods were developed for identification of pCQAs based on target binding affinity. An exemplary affinity fractionation method is illustrated in FIG. 13A and FIG. 13B. A protein of interest, for example an antibody, was subjected to affinity enrichment using a target, for example an antigen, as shown in FIG. 13A. An affinity column is first activated using HCl, followed by coupling to a target, equilibration using a buffer at a pH of about 8, and finally loading the protein of interest. An elution buffer was added at a varying pH to elute bound protein of interest in order of increasing affinity to the target. Fractions were then collected, with each fraction representing variants of the protein of interest with modified affinity to the target. FIG. 13B shows an exemplary pH gradient and elution of a protein of interest into seven collected fractions.

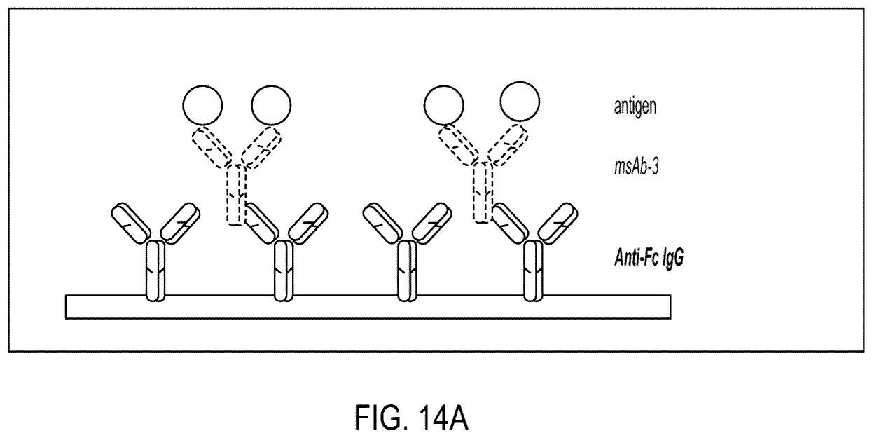

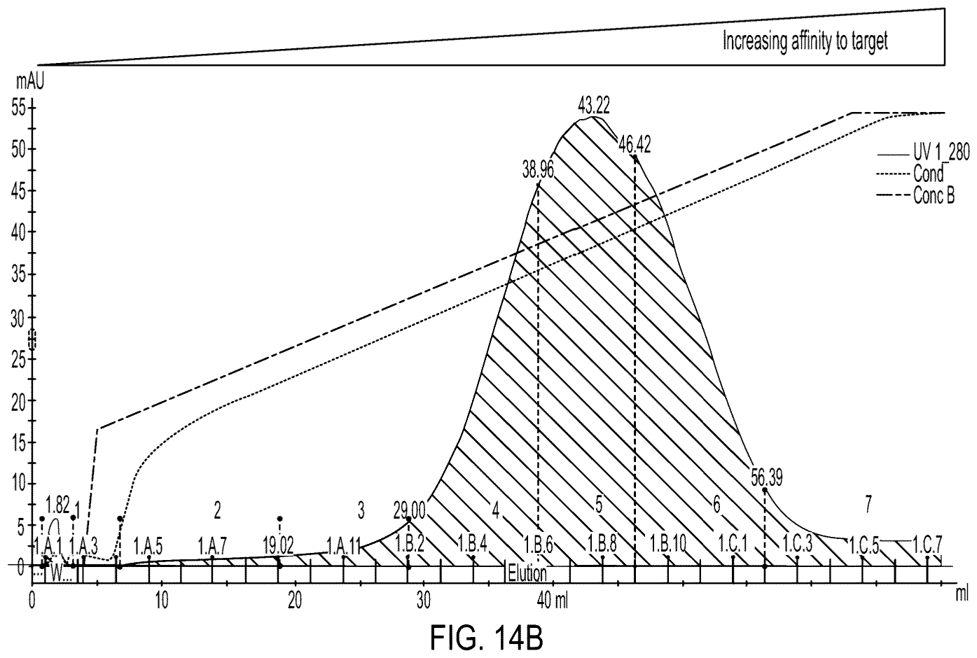

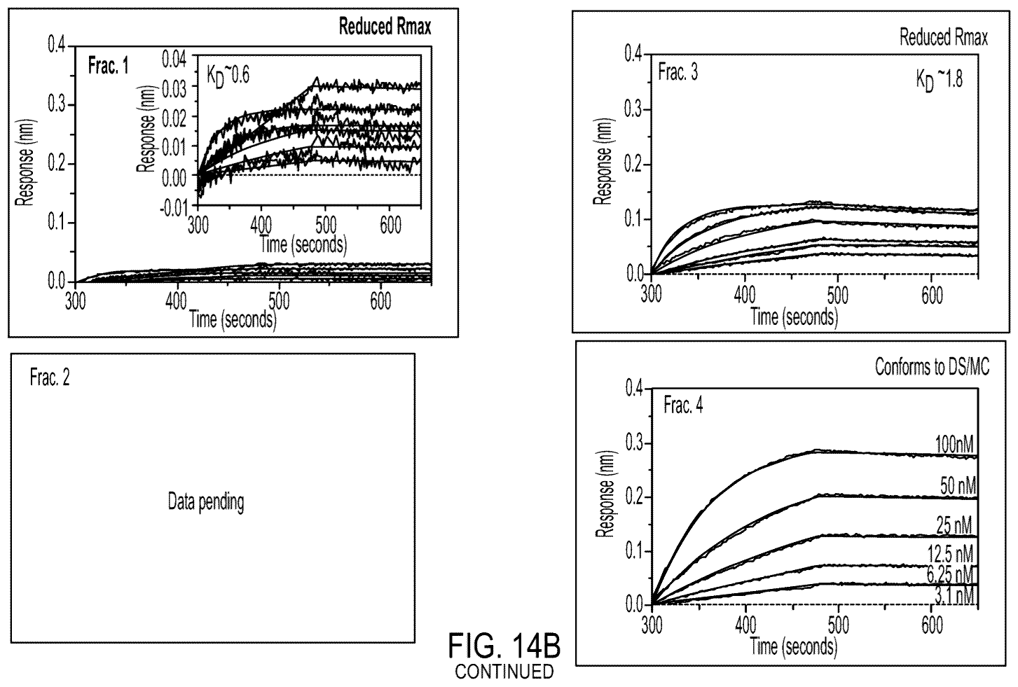

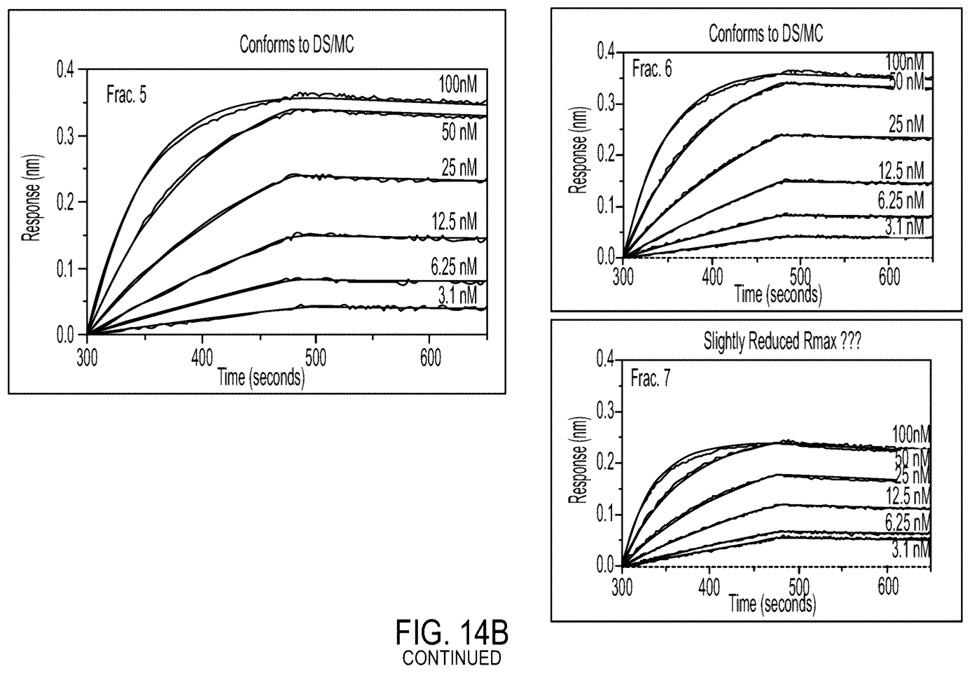

An in vitro target-binding assay may be used to verify the enrichment of fractions of the protein of interest that have modified affinity to a target ligand. Affinity enrichment was confirmed using biolayer interferometry (BLI). An Octet® BLI platform was employed. An illustration of the BLI format is shown in FIG. 14A. A monospecific antibody, msAb-3, was immobilized on a surface conjugated to anti-Fc IgG. The msAb-3 target antigen was introduced, and binding of msAb-3 to the antigen was measured using BLI. Binding efficacy of each of the collected affinity fractions was compared, as shown in FIG. 14B. Binding for each of the main peak fractions was found to correspond to the results from unfractionated drug substance (DS). Each of the other fractions was found to have reduced or slightly reduced Rmax, the theoretical maximum response for analyte binding. The BLI results validated the affinity enrichment fractionation.

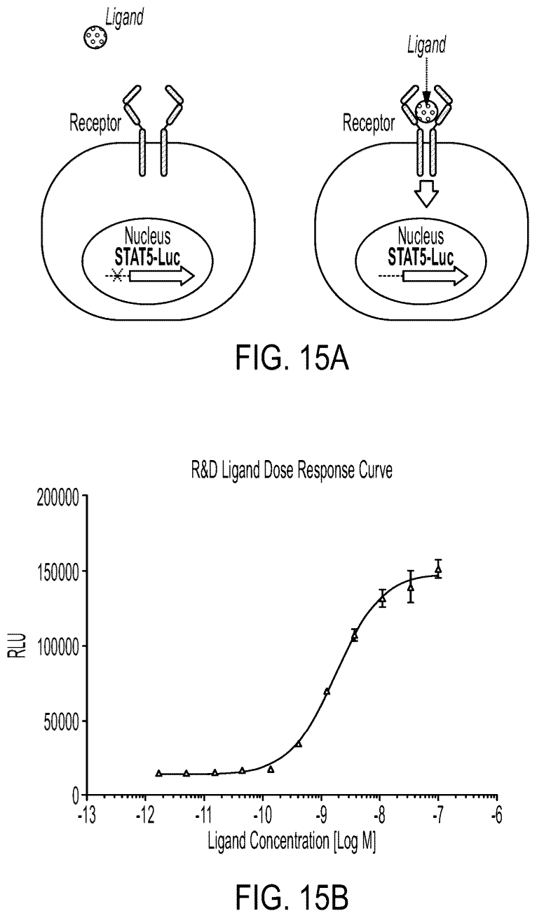

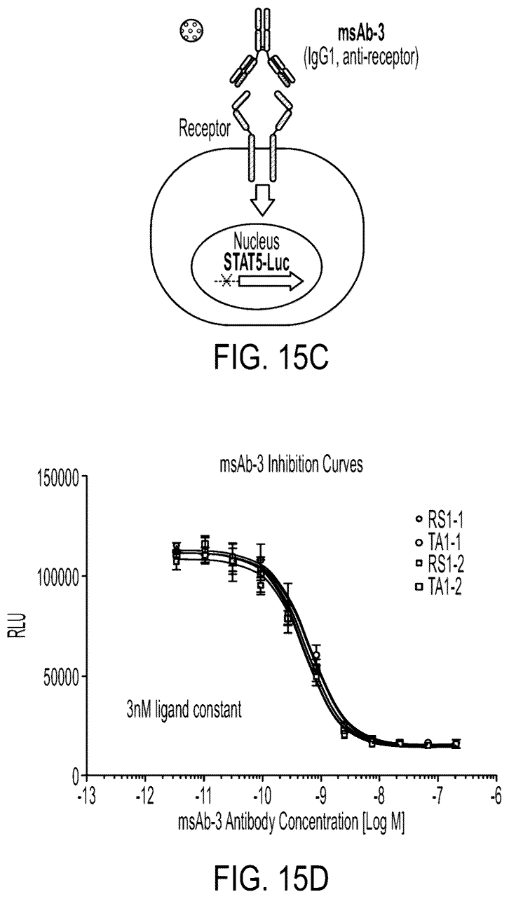

Next, a cell-based bioassay was employed to determine the connection between target binding efficacy and therapeutic protein potency. An exemplary bioassay is illustrated in FIG. 15A. The antigen for msAb-3, which is a cell surface receptor, was expressed on the surface of a HEK293 cell. Binding of the corresponding ligand to the receptor causes expression of luciferase, generating a measurable luminescent signal. A dose response curve for the ligand is shown in FIG. 15B. Addition of msAb-3 blocks binding of the ligand to the receptor, thereby reducing luciferase signal, as illustrated in FIG. 15C. An exemplary inhibition curve for msAb-3 is shown in FIG. 15D.

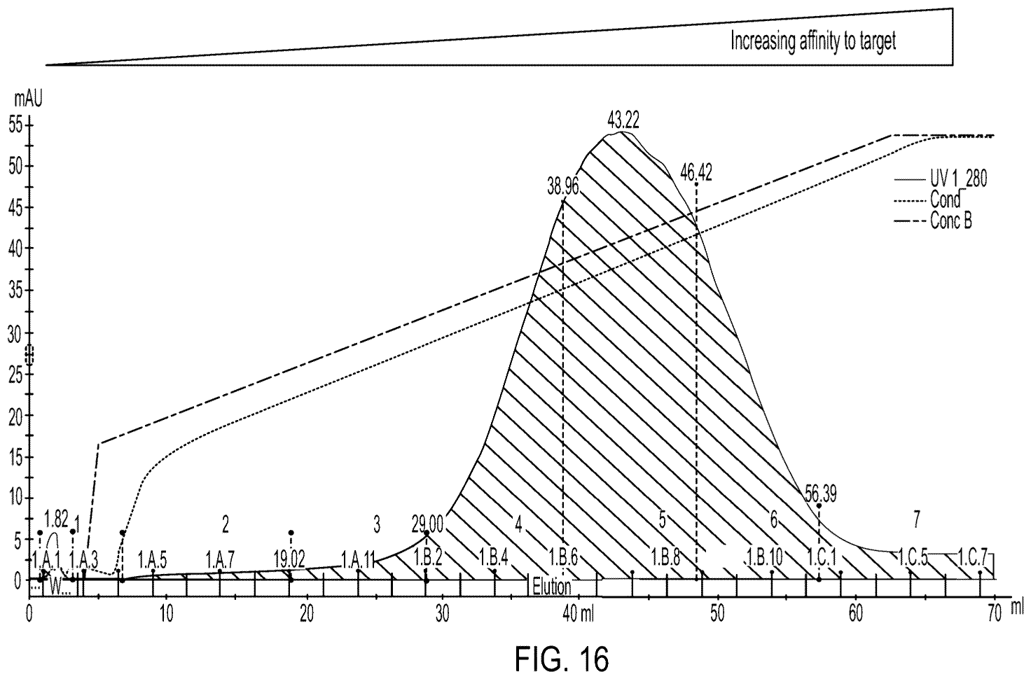

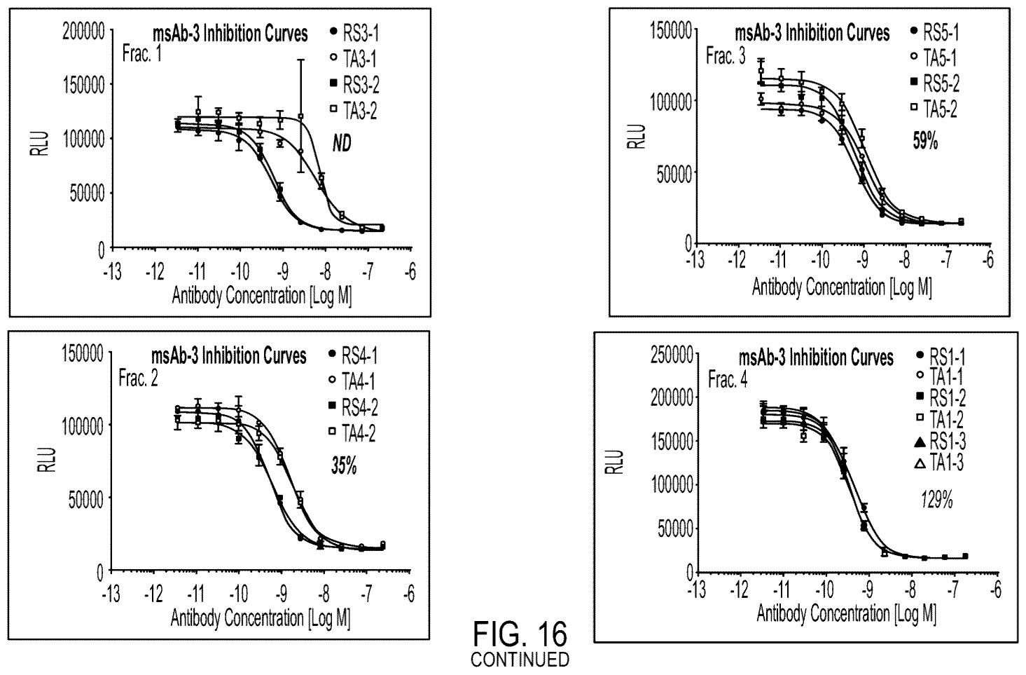

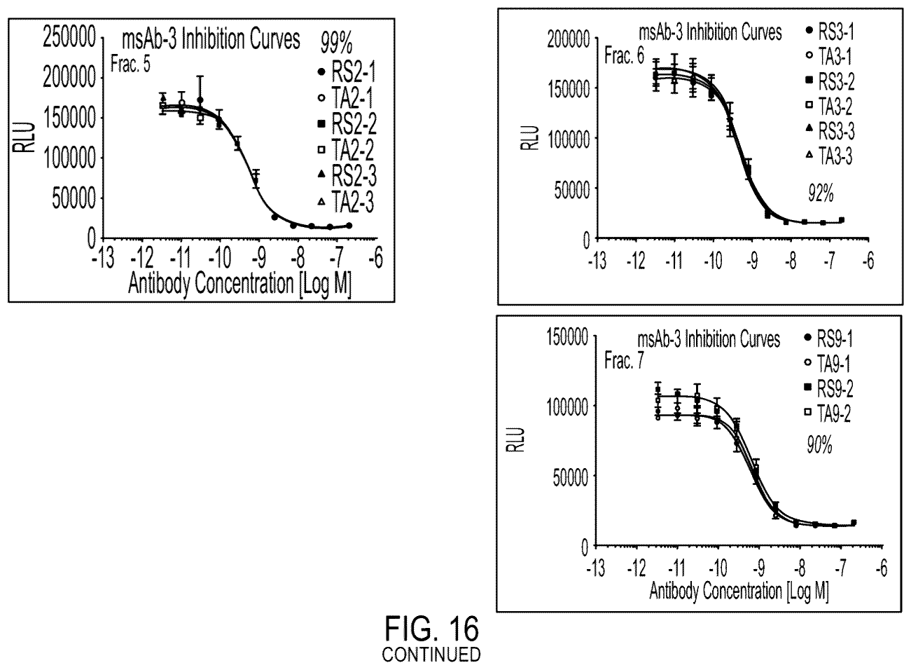

A comparison of the potency of msAb-3 from each of the affinity enrichment fractions as measured by the cell-based bioassay is shown in FIG. 16. msAb-3 from the main peak was shown to have the greatest potency, validating that variants separated from the main peak by affinity enrichment are variants with reduced potency.

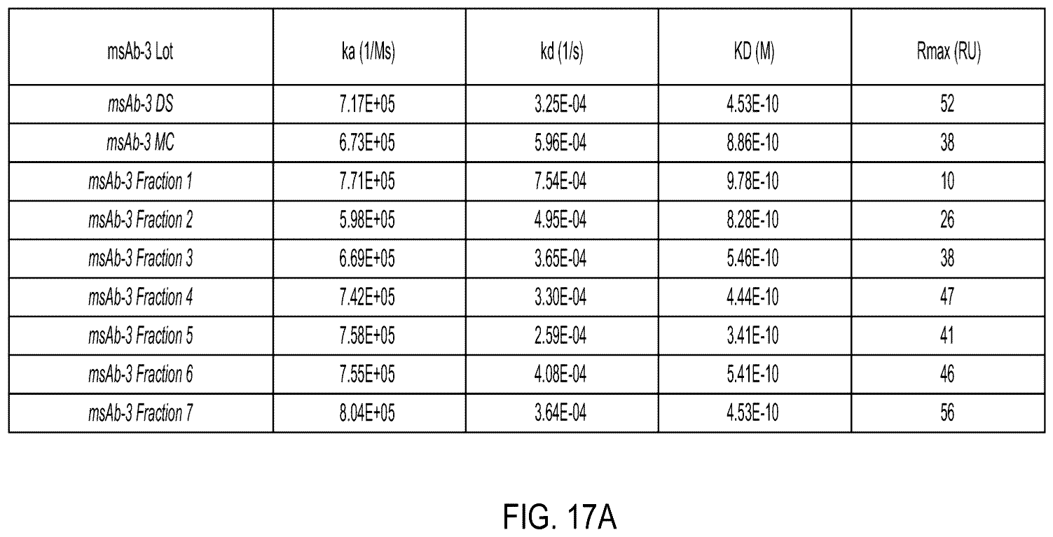

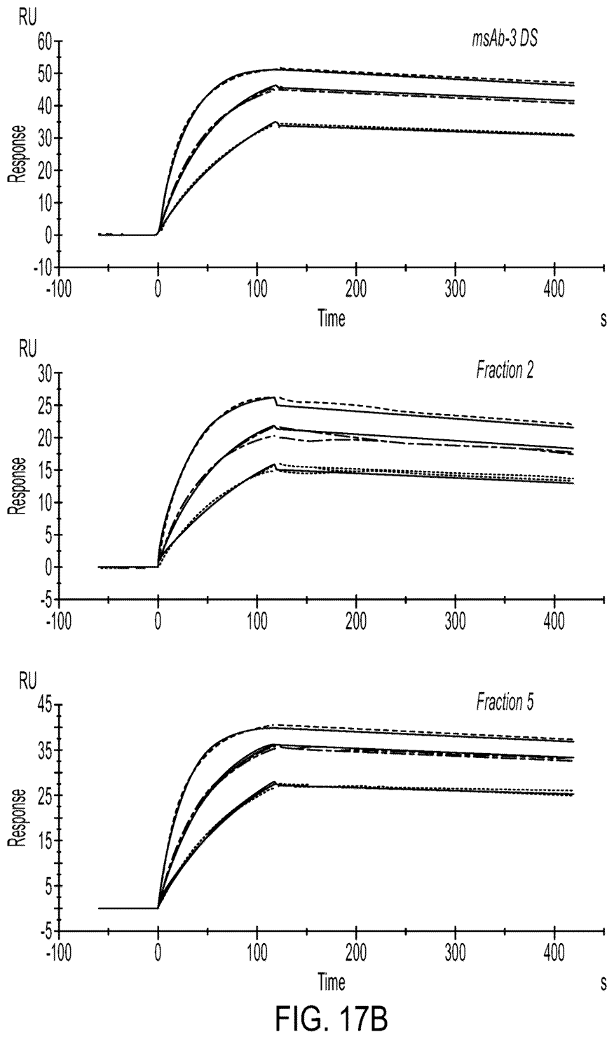

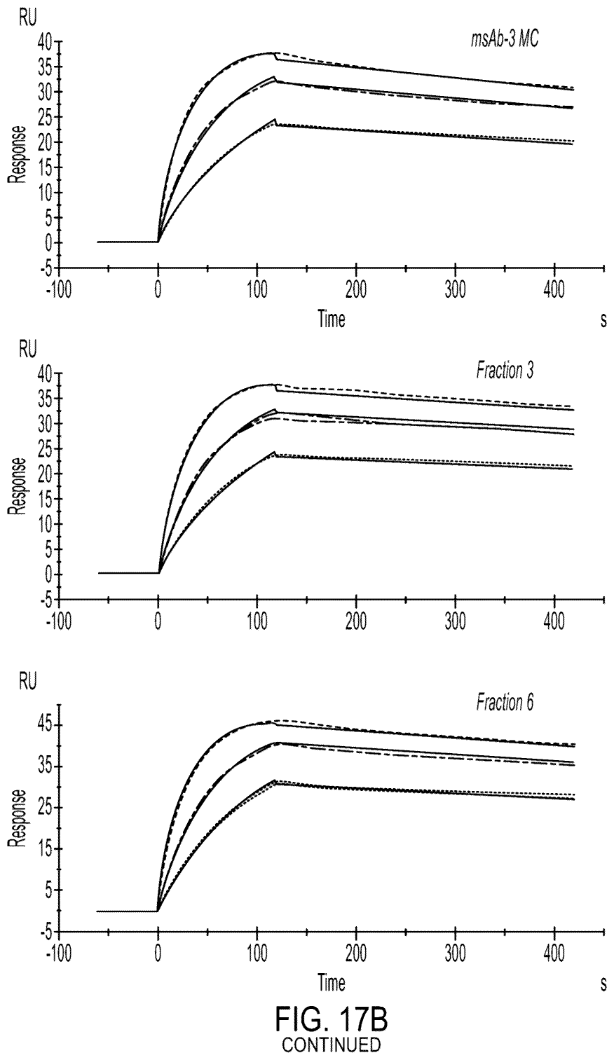

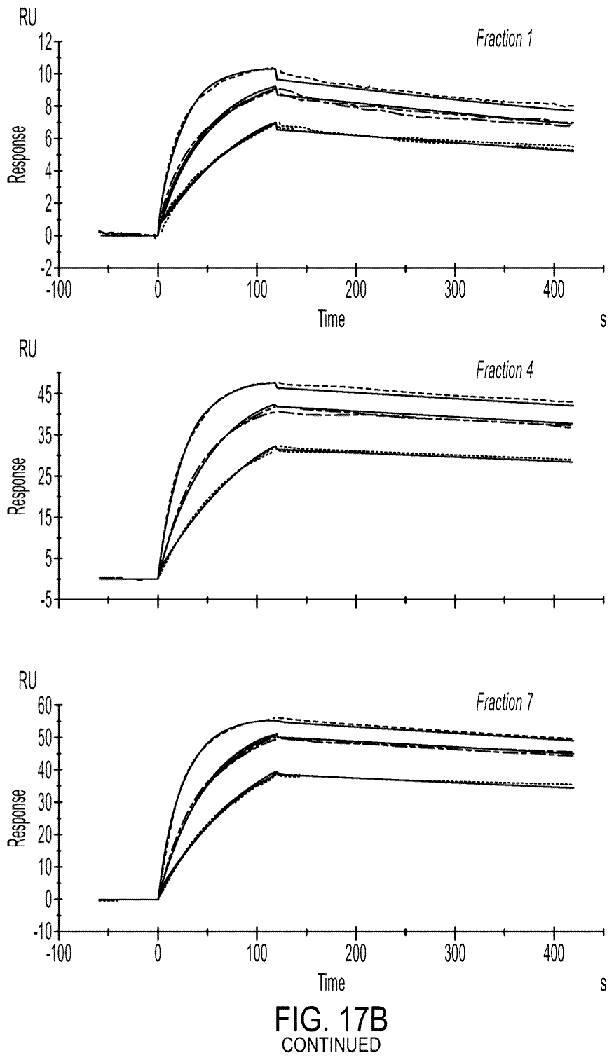

The affinity enrichment fractions were further characterized using surface plasmon resonance (SPR) analysis to measure the binding characteristics of each msAb-3 fraction to its target. A summary of Kₐ (1/ms), K_{d} (1/s), K_{D} (M), and Rmax (RU) for each fraction is shown in FIG. 17A, validating that fractions that eluted later from the affinity column had greater binding capacity (Rmax) than fractions that eluted earlier. FIG. 17B shows exemplary SPR sensorgrams for each fraction, with the lower line in each graph showing a response to 12.5 nM target, the middle line showing a response to 25 nM target, and the higher line showing a response to 50 nM target.

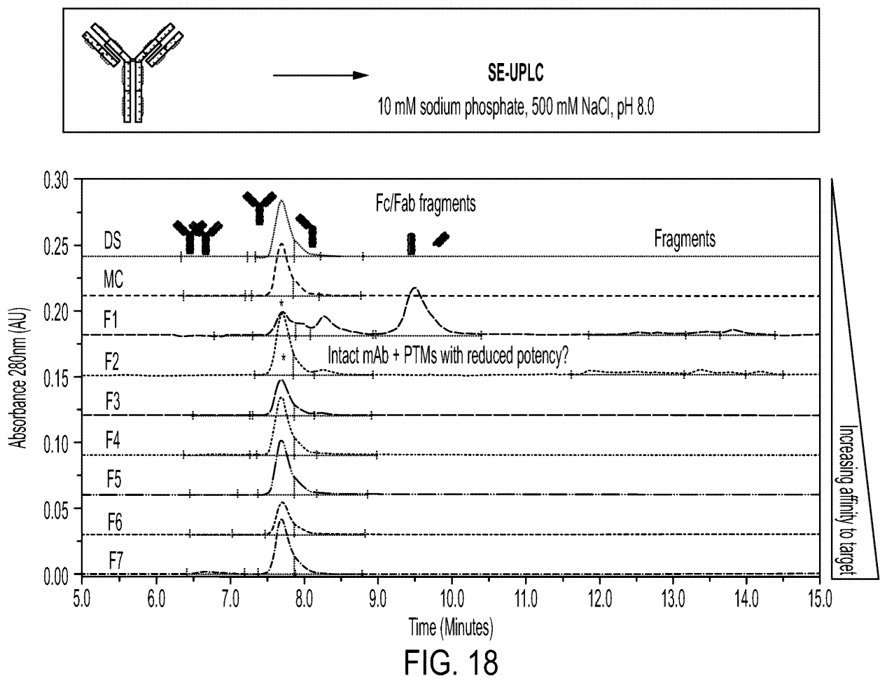

In order to characterize size variants separated by the affinity enrichment method of the present invention, the affinity enrichment fractions were subjected to size exclusion-ultra performance liquid chromatography (SE-UPLC). Size variants caused by PTMs or fragmentation were separated for each fraction, as shown in FIG. 18. Each fraction demonstrated a distinct profile indicating distinct fragments and PTMs.

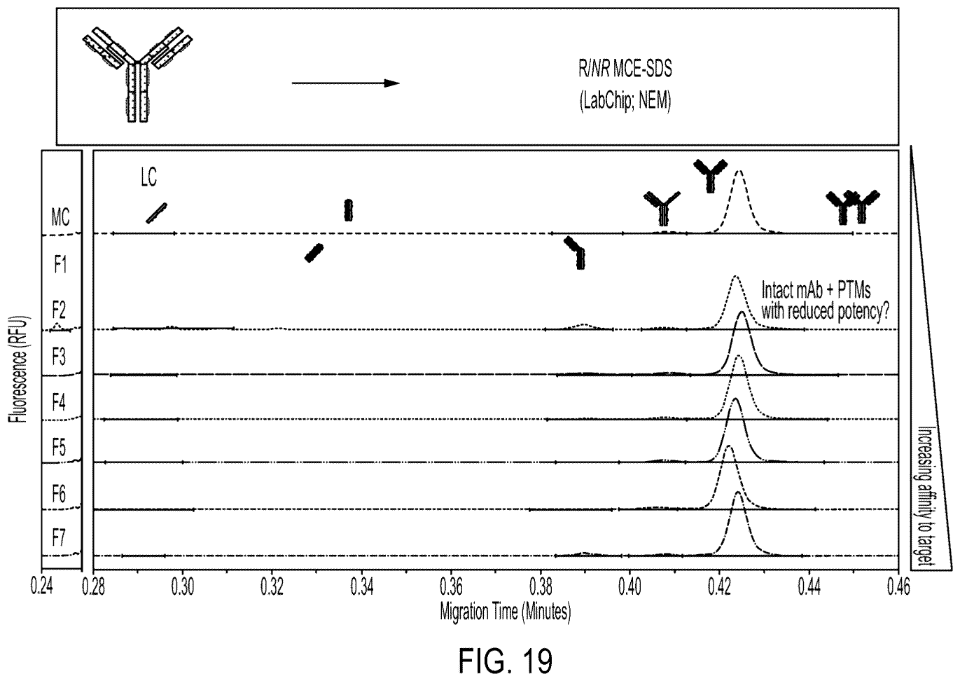

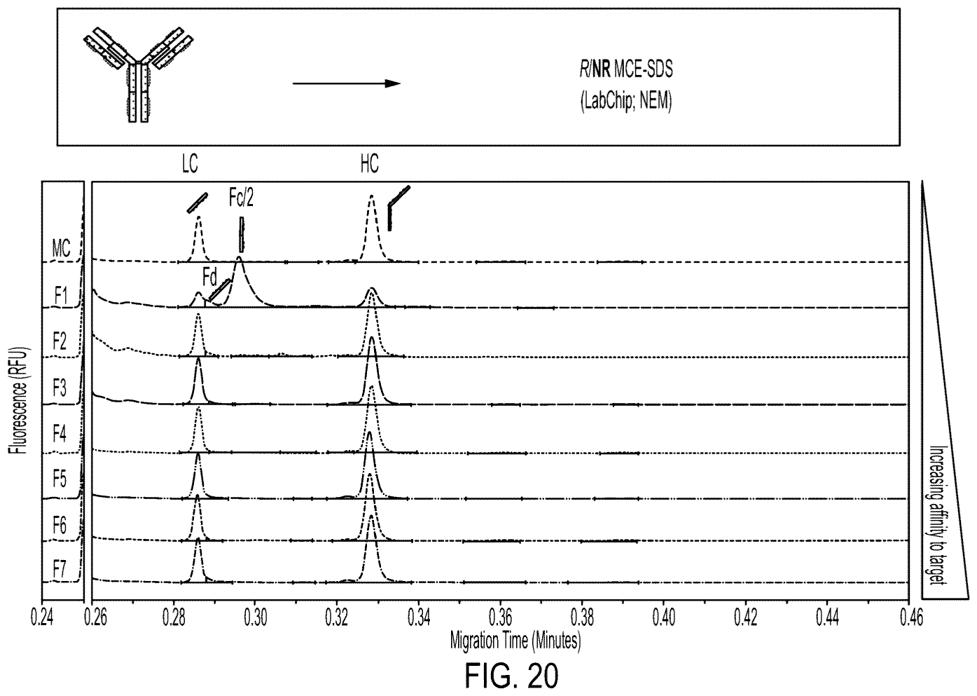

Variants were further characterized using non-reducing microchip electrophoresis (MCE-SDS), as shown in FIG. 19, and reducing MCE-SDS, as shown in FIG. 20, each of which yielded a set of profiles for affinity enrichment fractions showing a presence of distinct protein variants in each fraction.

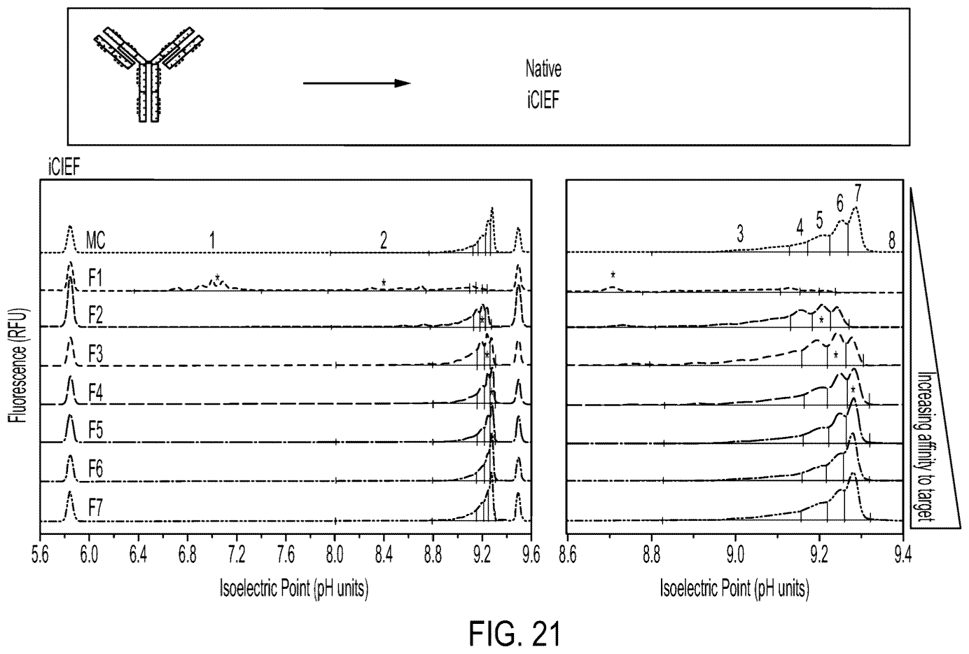

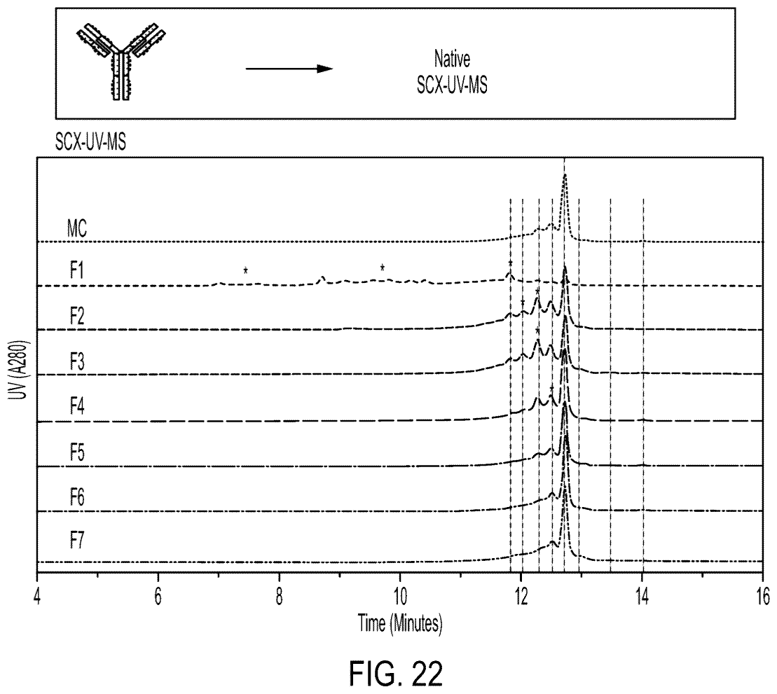

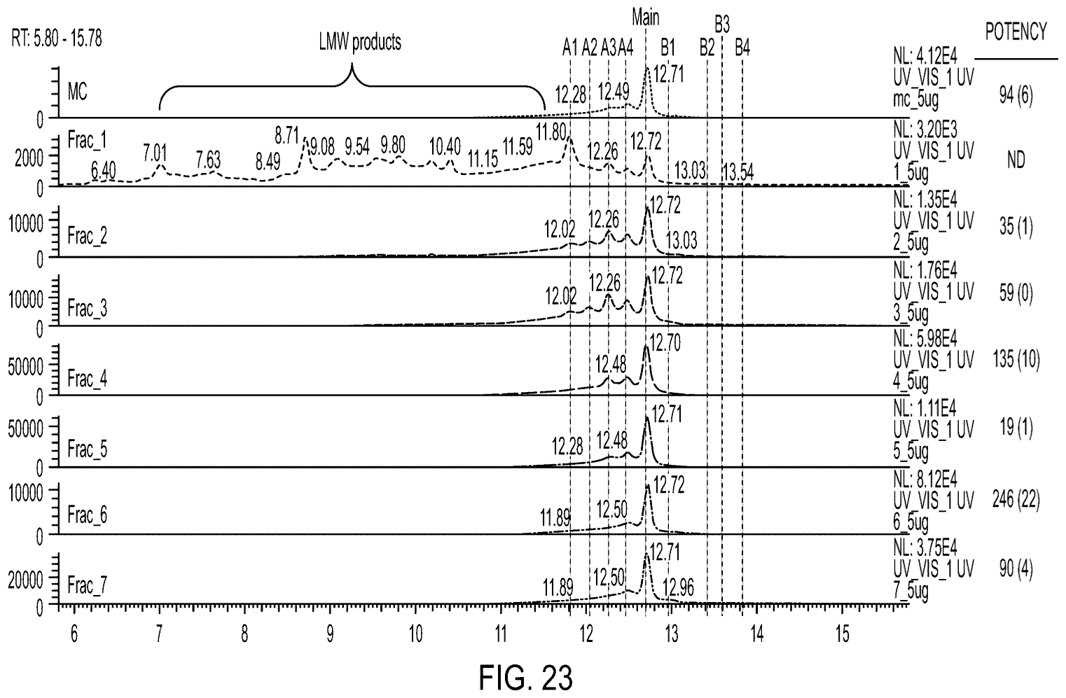

Charge variants separated by the affinity enrichment method of the present invention were characterized by native imaged capillary isoelectric focusing (iCIEF), as shown in FIG. 21, and by native strong cation exchange chromatography coupled with ultraviolet detection and mass spectrometry (SCX-UV-MS), as shown in FIG. 22. Native SCX-UV analysis revealed enrichment of acidic charge variants in fractions 1-3 and low molecular weight (LMW) species in fraction 1, as shown in FIG. 23.

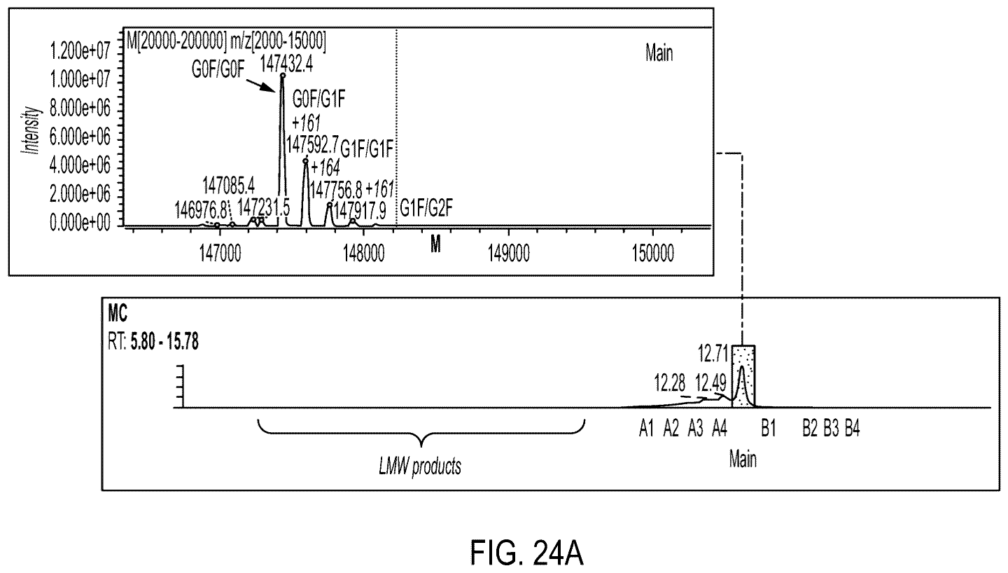

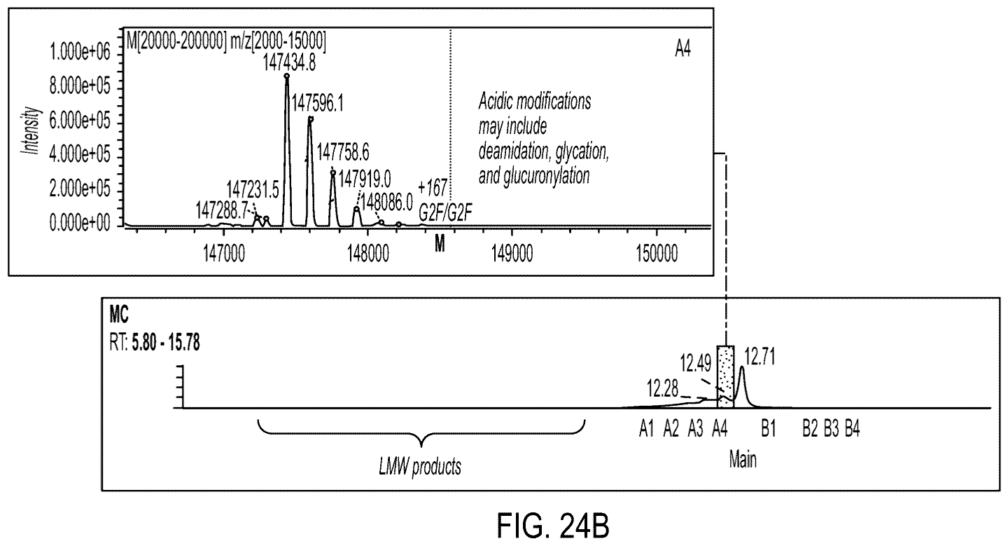

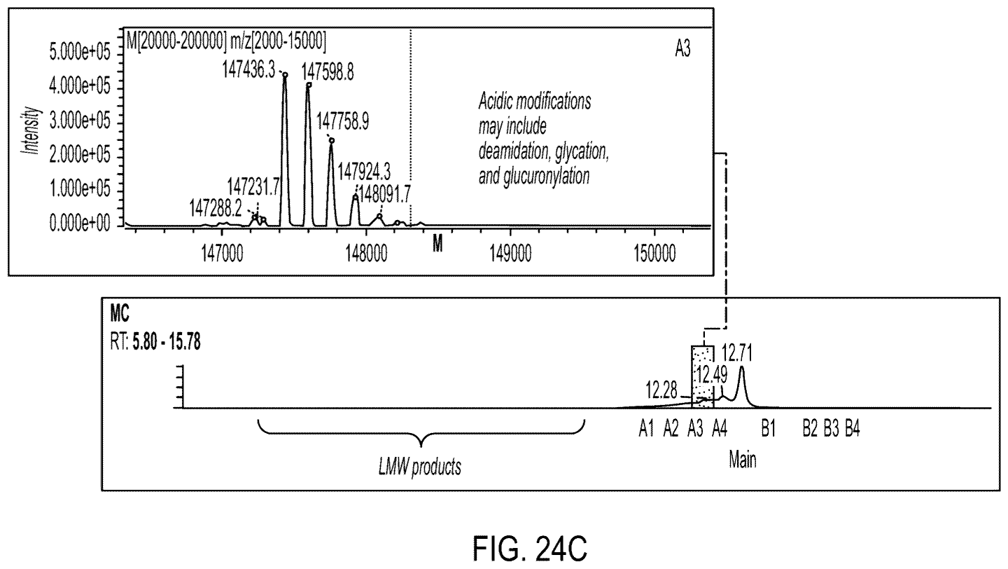

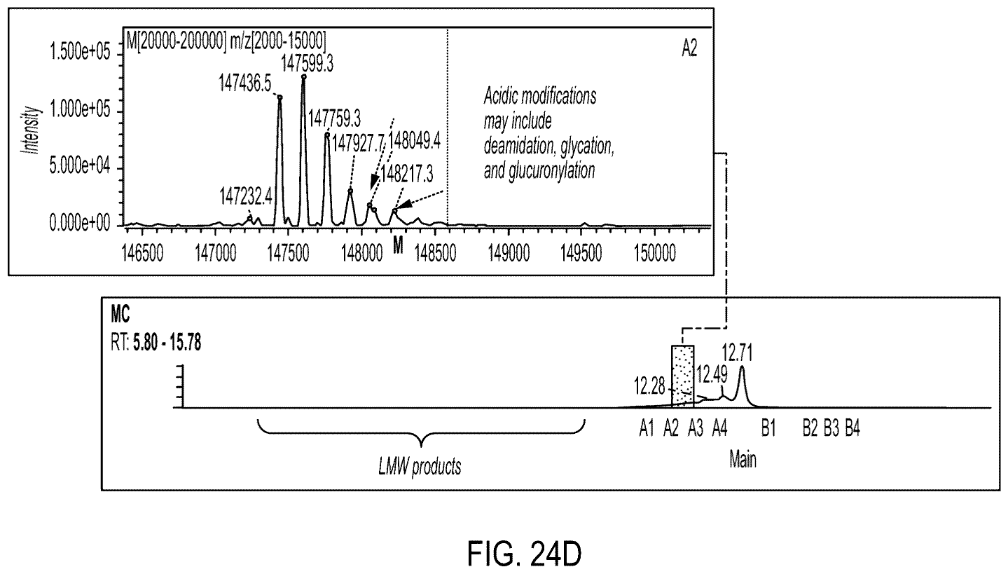

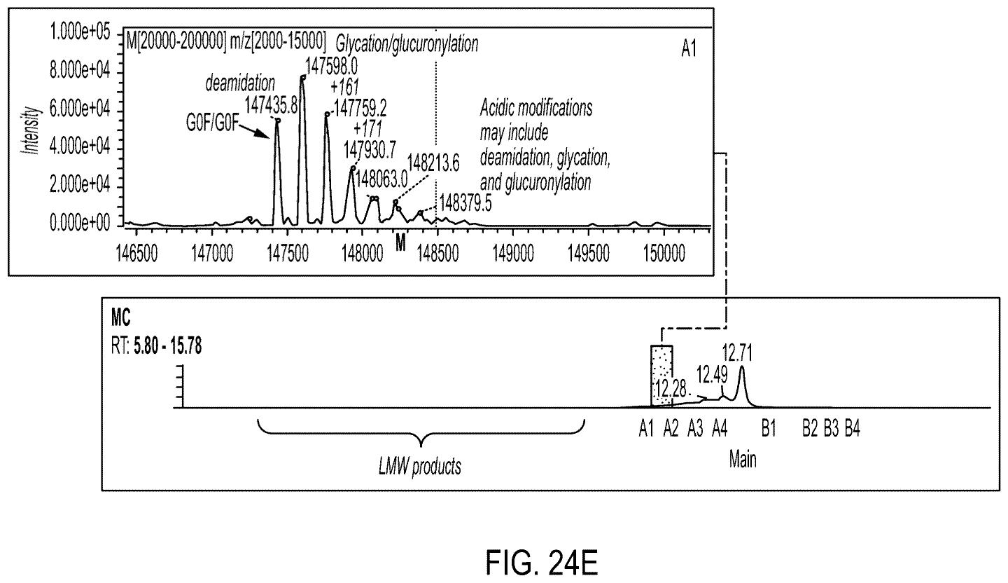

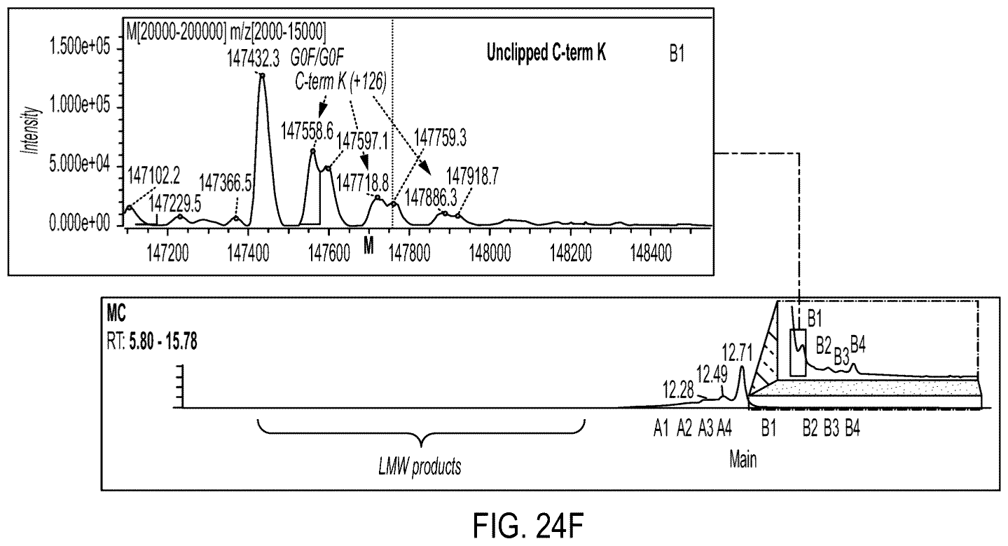

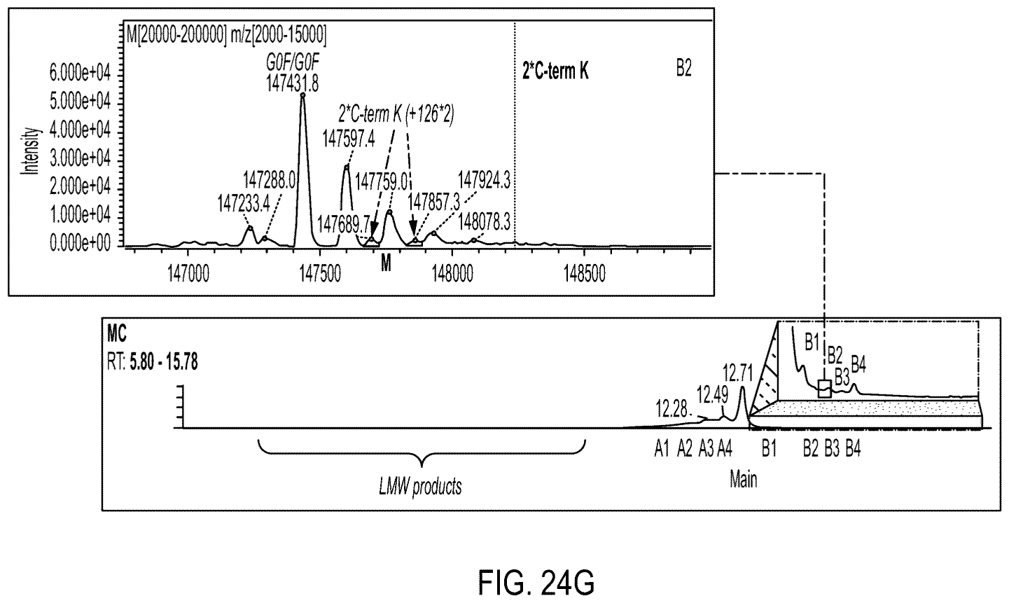

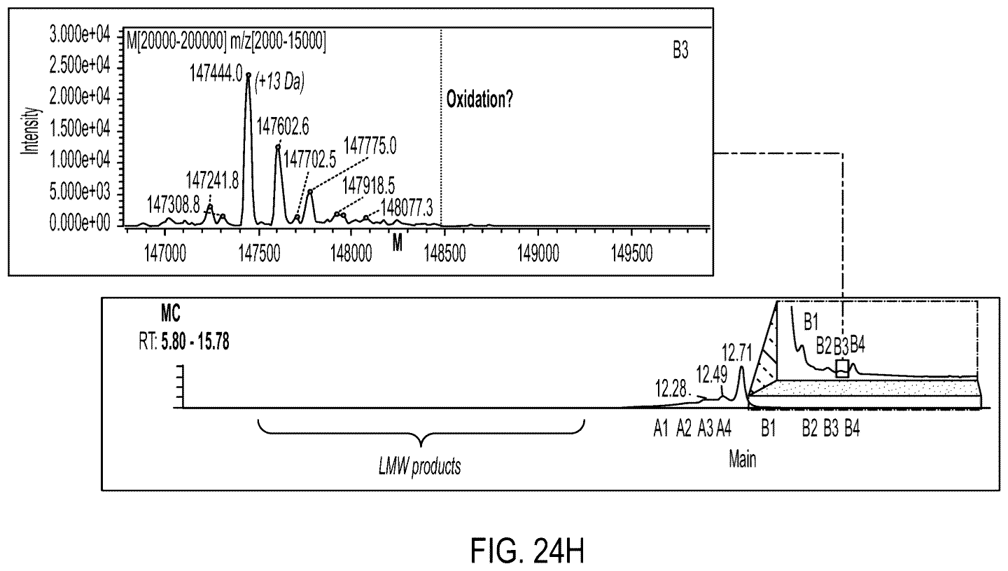

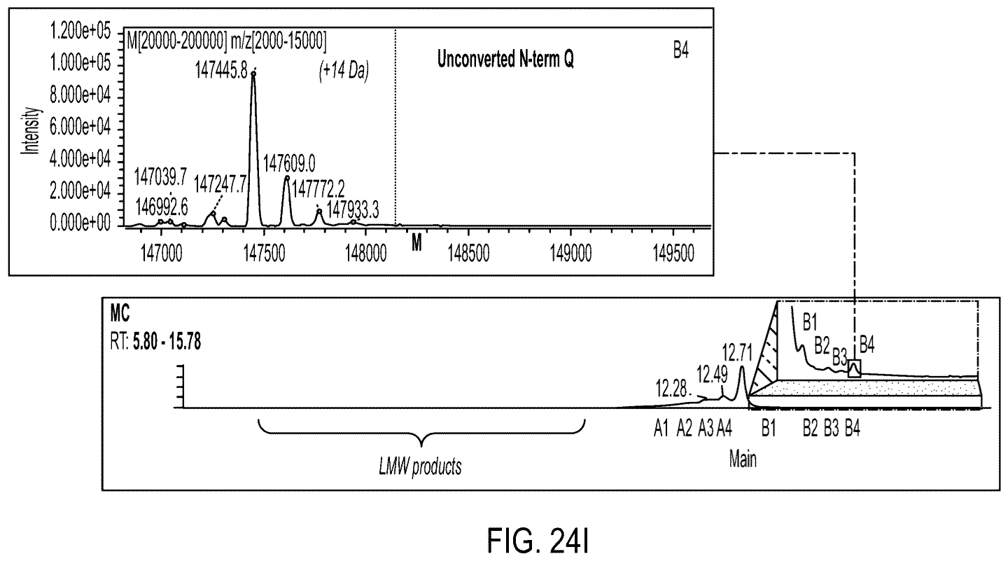

Using MS, post-translational modifications (PTMs) associated with each charge variant of each affinity enrichment fraction were characterized. The main charge species in msAb-3 drug substance is the most homogeneous for the G0F/G0F variant, as shown in FIG. 24A. Acidic PTMs in DS samples mostly consisted of deamidation, glycation, and glucuronylation. Peaks A4 and A3 had a change in distribution of glycosylation variants, but no new peaks corresponding to unique PTMs, as shown in FIG. 24B and FIG. 24C respectively. Peak A2 had slightly higher heterogeneity, and peak A1 showed the highest heterogeneity, as shown in FIG. 24D and FIG. 24E respectively.

Glycosylation variants comprised the majority of peaks detected. Basic PTMs in the DS samples mostly consisted of unclipped C-terminal lysines plus N-terminal pyroglutamate formation. Peak B1 had new peaks corresponding to 1× C-terminal lysine, as shown in FIG. 24F. Peak B2 had very minor new peaks corresponding to 2× C-terminal lysines, as shown in FIG. 24G. Peak B3 has a major peak corresponding to G0F/G0F plus an unknown modification, and some heterogeneity, as shown in FIG. 24H. Peak B4 is shown in FIG. 24I.

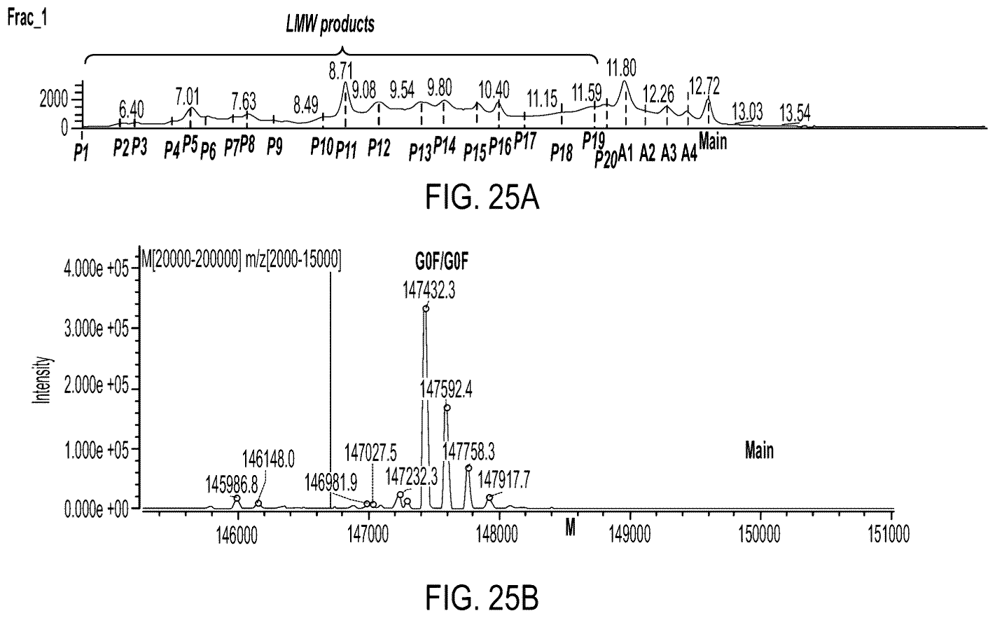

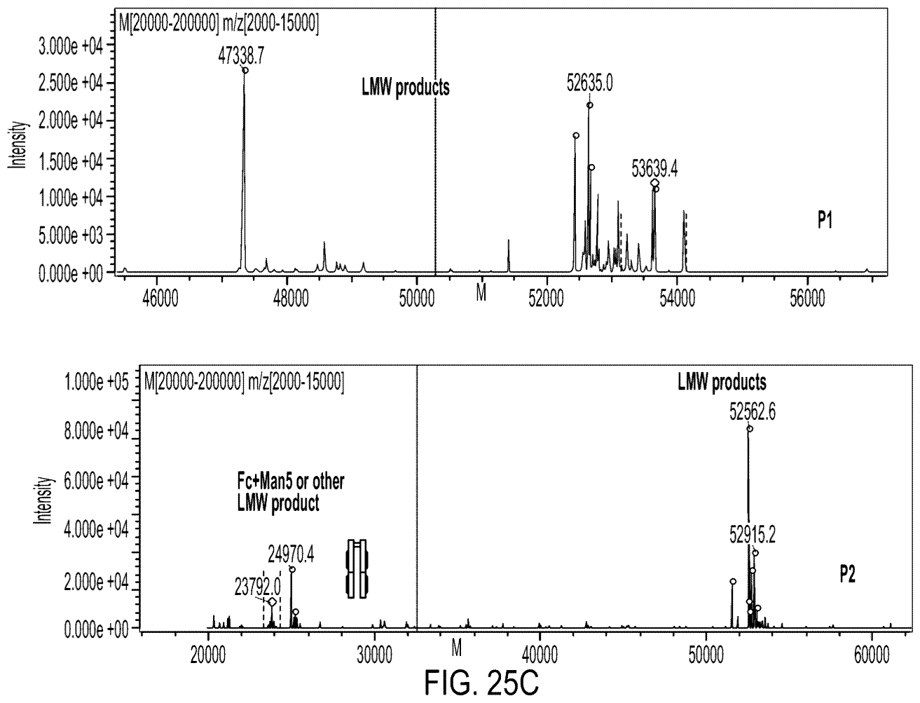

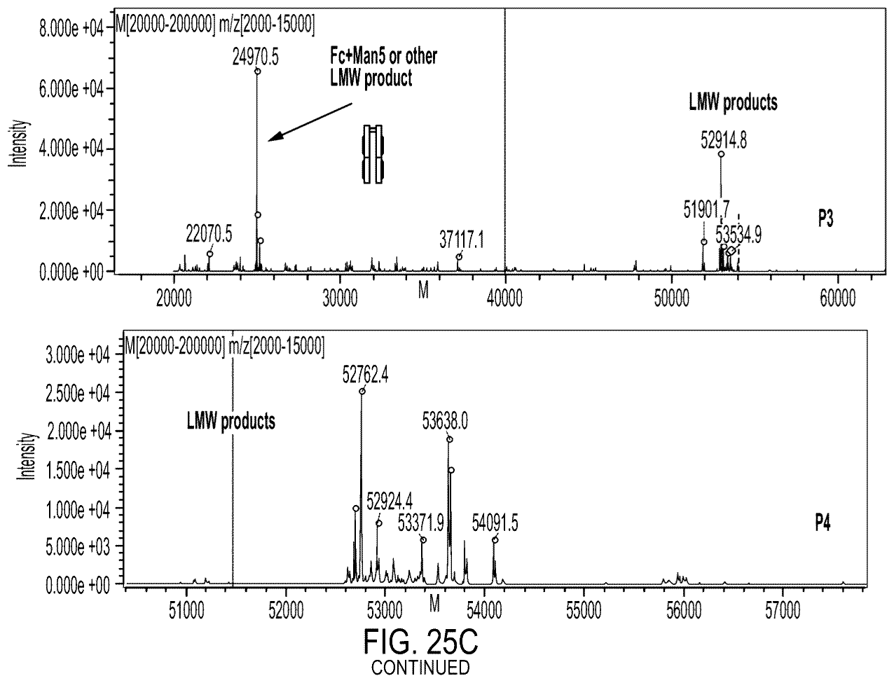

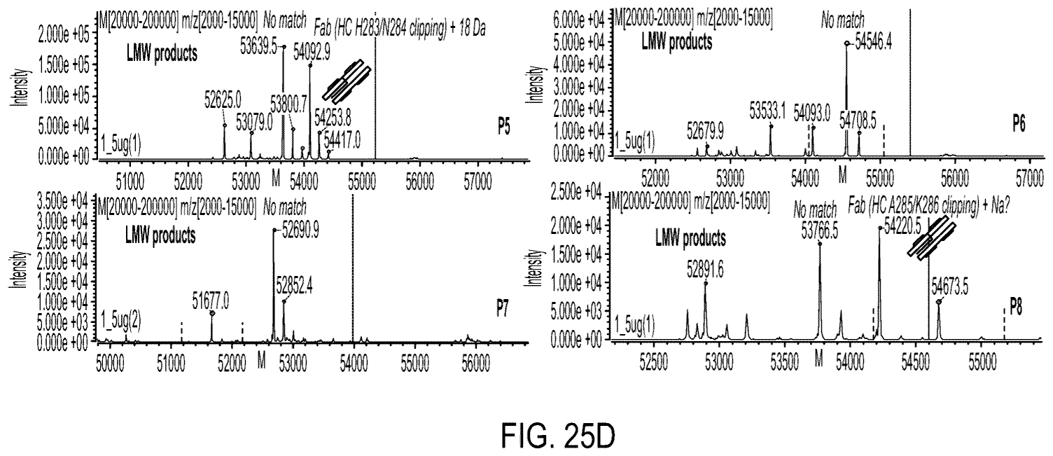

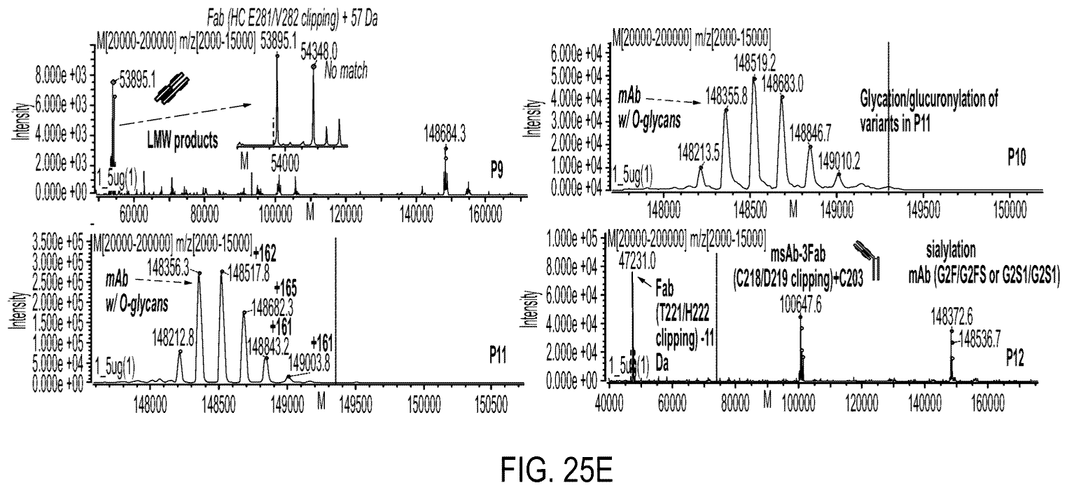

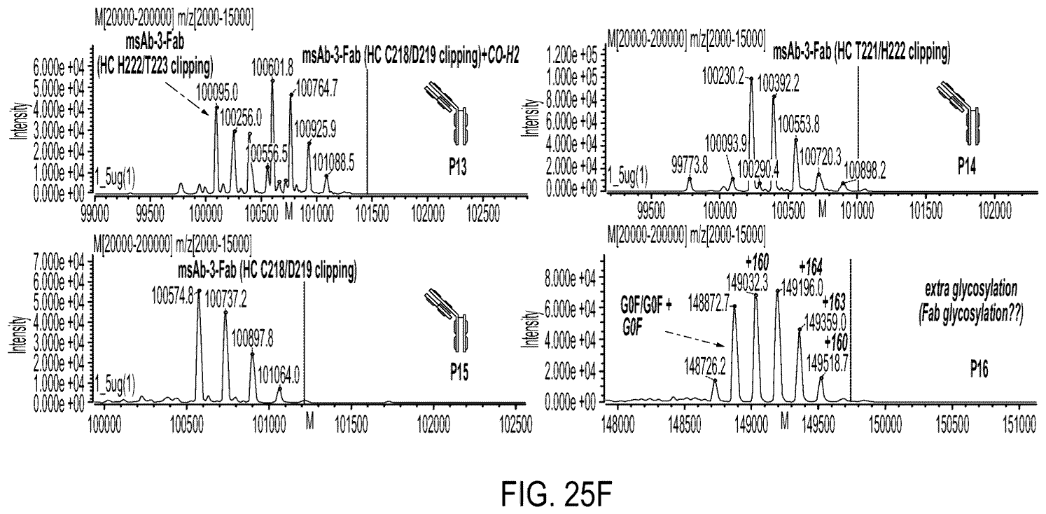

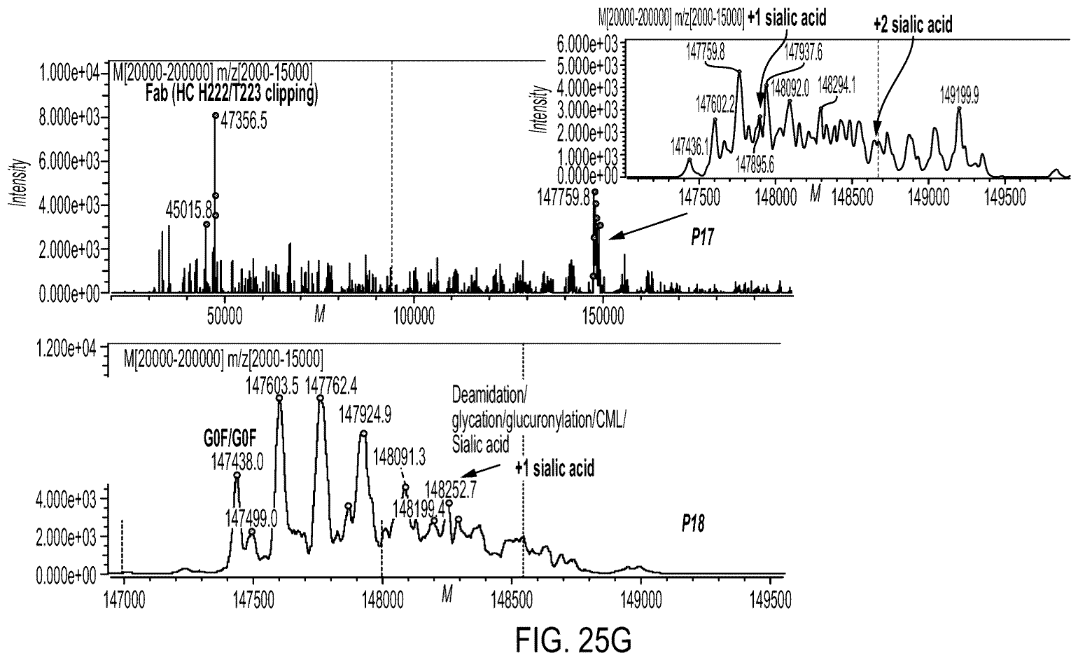

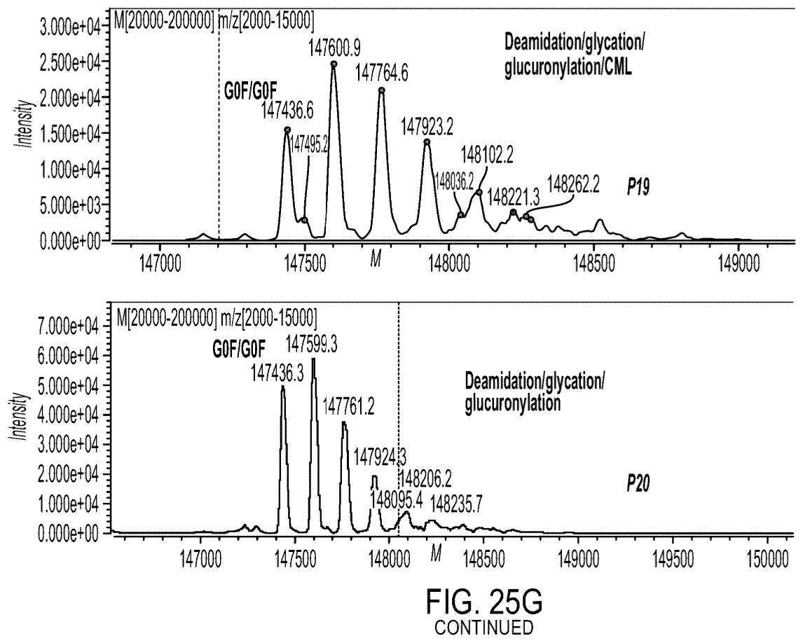

As described earlier and illustrated again in FIG. 25A, SCX-UV analysis of fraction 1 showed enrichment for low molecular weight products and acidic charge variants. Therefore, MS analysis was used to characterize fraction 1 variants. In addition to common acidic variants such as deamidation, glycation, and glucuronylation, fraction 1 also contains sialylation, Fab glycosylation, and multiple LMW products likely generated by degradation. Mass spectra for fraction 1 main peak and peaks 1-20 are shown in FIGS. 25B-25G, showing the identification of diverse antibody variant species for each peak separated by SCX from affinity enrichment fraction 1.

As described above, using intact native SCX analysis, different profiles were observed across msAb-3 affinity enrichment fractions 1-7, suggesting that they contained different charge variants. According to the characterization of fraction 1, degradation products were observed which might impact antibody binding. Acidic modifications, including deamidation, glycation, glucuronylation, Fab glycosylation, and sialylated Fc glycosylation, and basic modifications, including C-terminal lysines and N-terminal pyroglutamate, were observed in different samples, which can also affect binding.

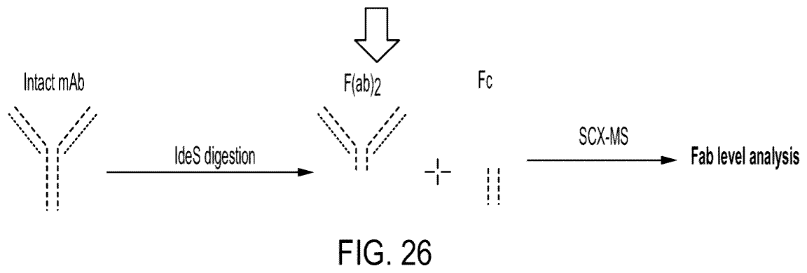

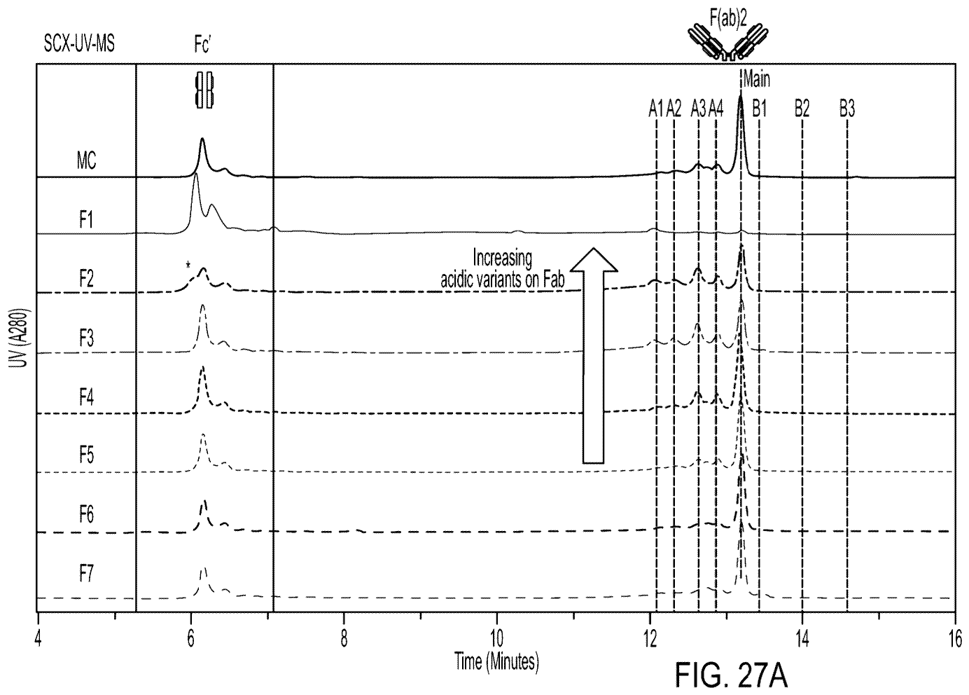

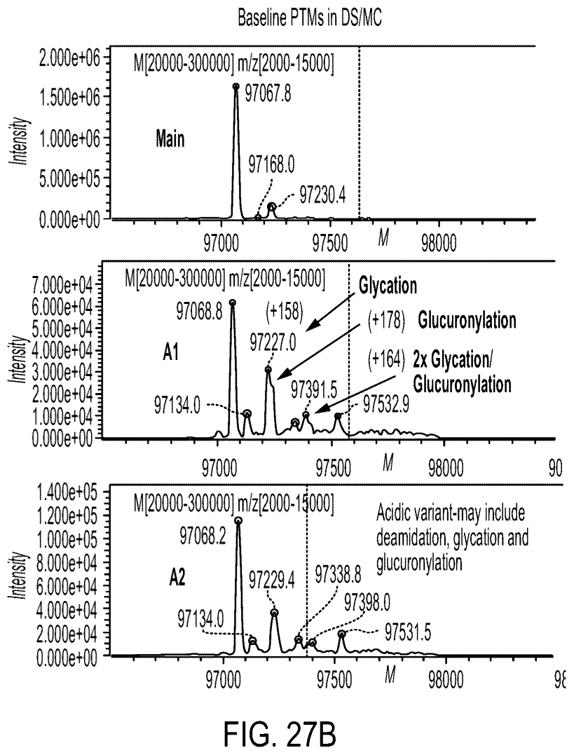

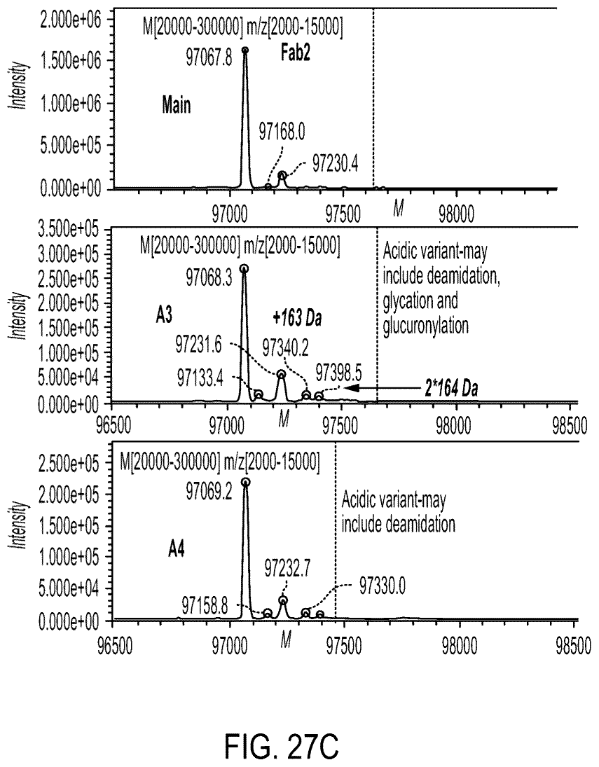

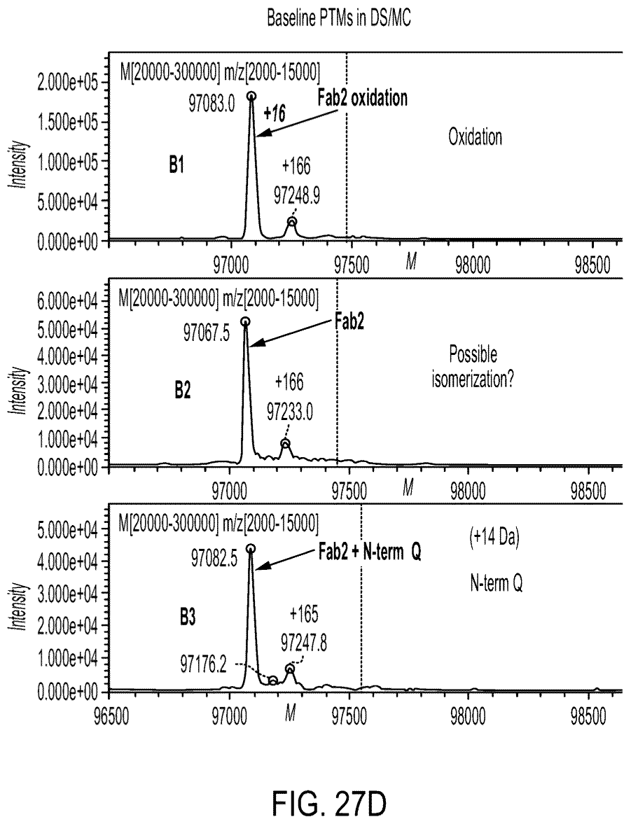

The location of degradations and modifications can be further investigated by subunit analysis. For example, prior to or following affinity fractionation, an antibody of interest can be digested into subunits using IdeS, papain, or pepsin, as illustrated using the example of IdeS in FIG. 26. Accordingly, SCX-UV-MS analysis of IdeS-digested subunits from msAb-3 affinity enrichment fractions was performed, as shown in FIG. 27A. Four acidic peaks and three basic peaks were identified. MS analysis was used to characterize PTMs in each of the charge variant peaks using msAb-3 drug substance, as shown in FIG. 27B, FIG. 27C, and FIG. 27D.

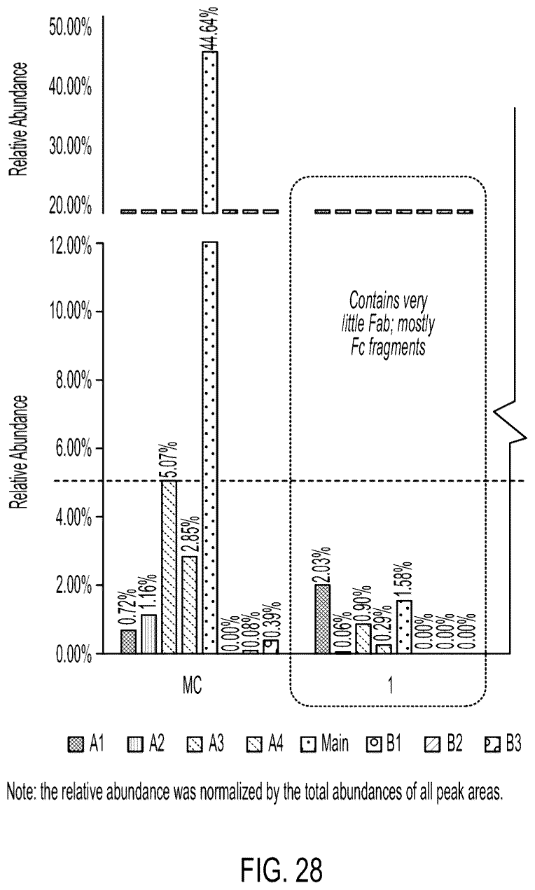

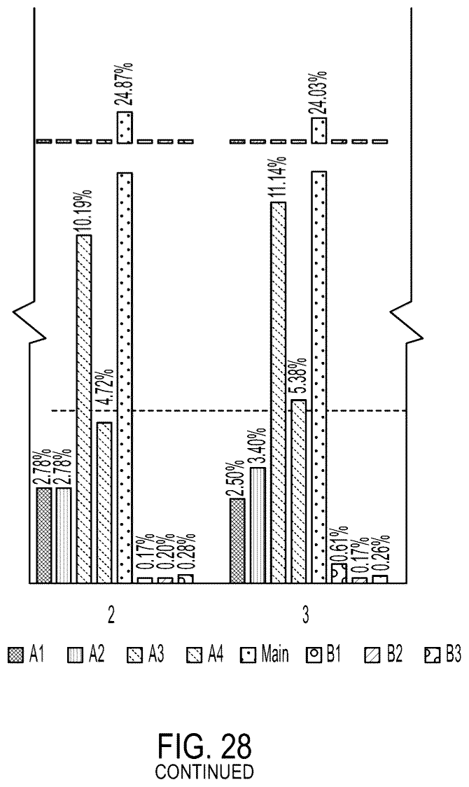

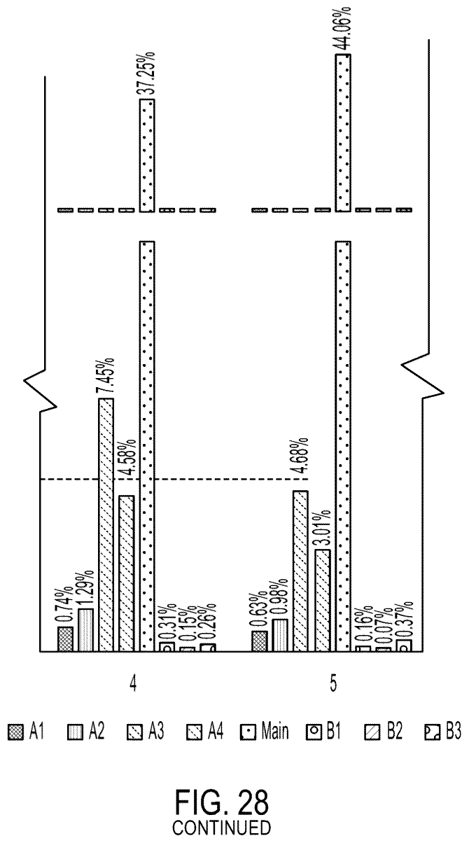

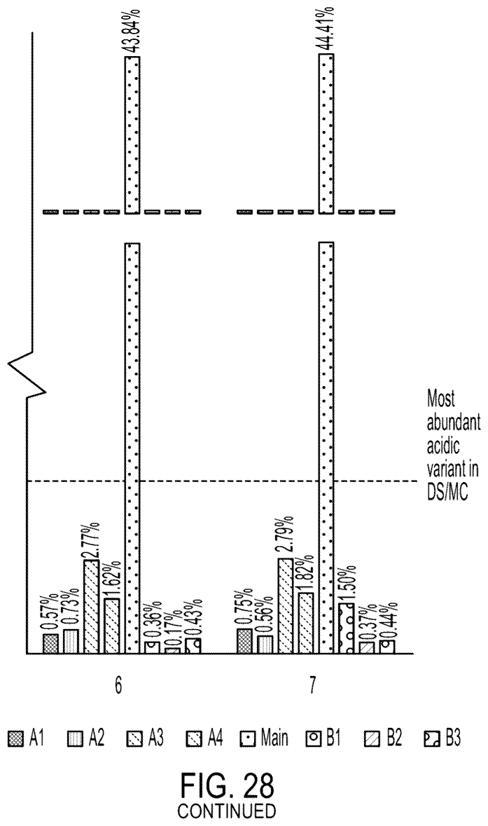

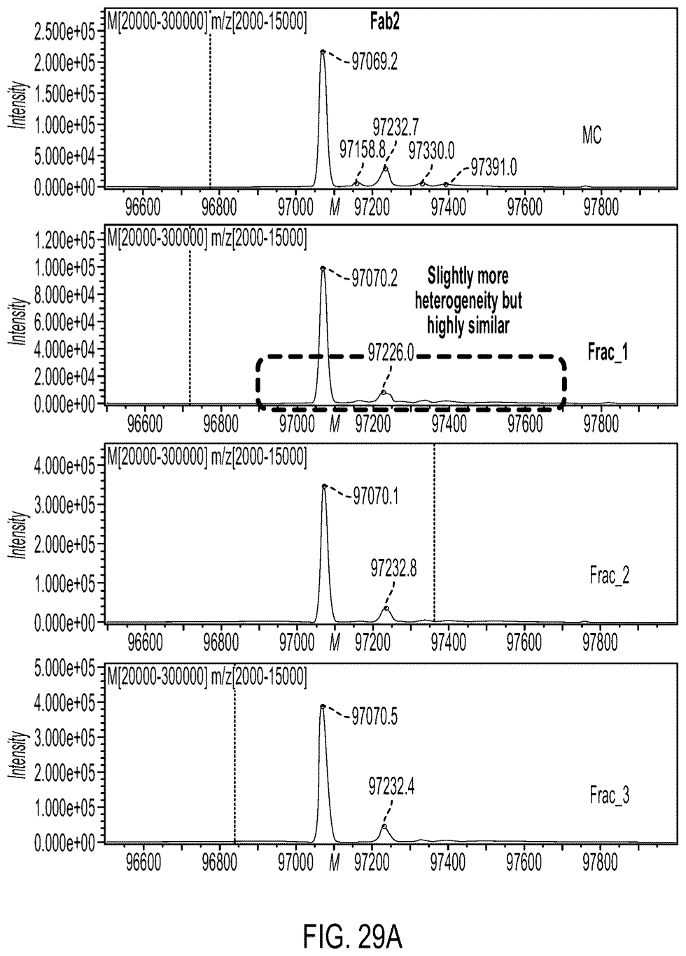

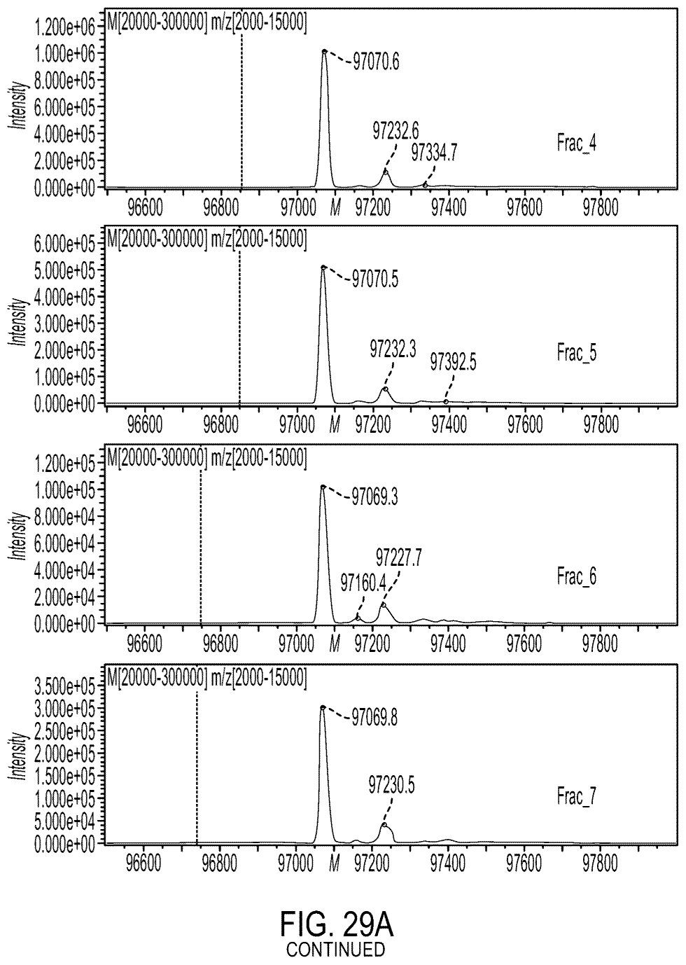

The relative abundance of each charge variant peak in Fab fragments for each affinity enrichment fraction are shown in FIG. 28. The relative abundance was normalized by the total abundances of all peak areas. Fraction 1 was found to contain mostly LMW species due to degradation of the antibody. Fractions 2 and 3 contained the highest level of acidic variants. Fraction 7 contained a slightly higher level of B1 and B2 peaks.

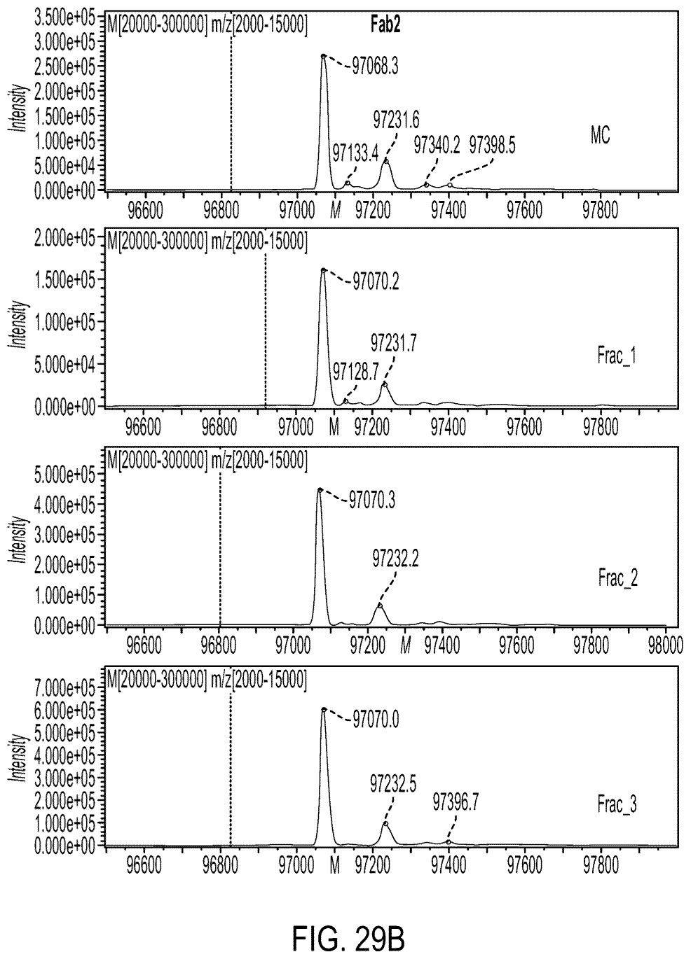

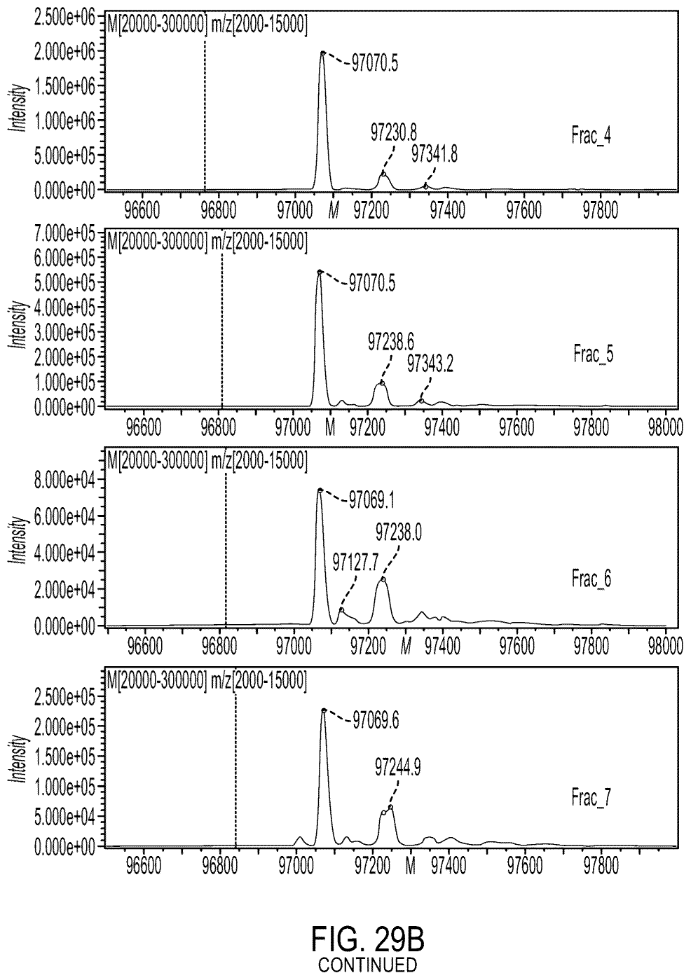

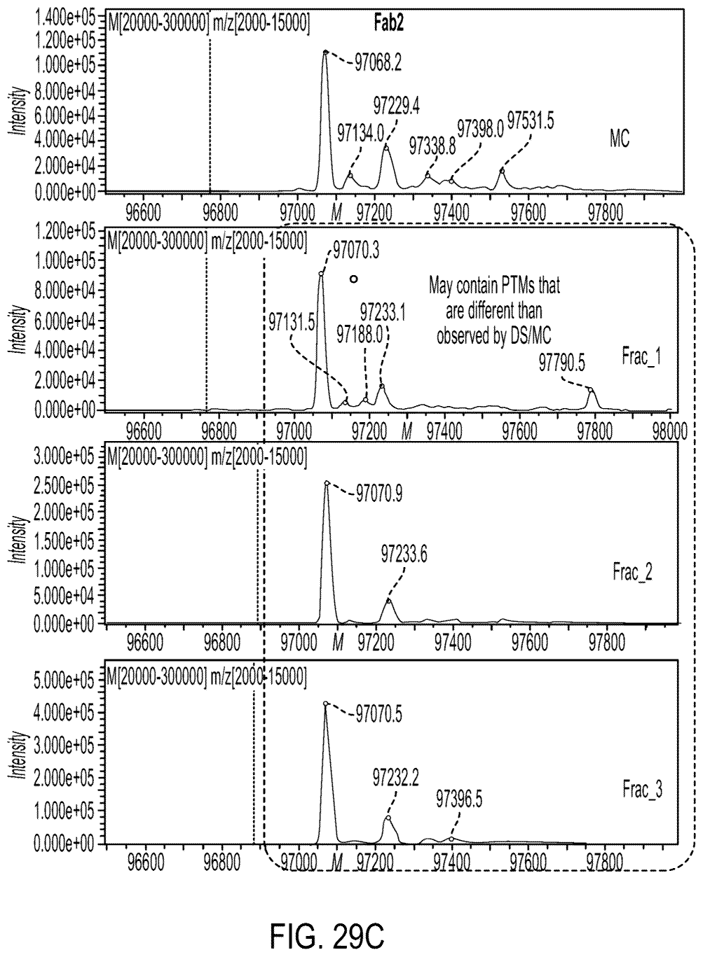

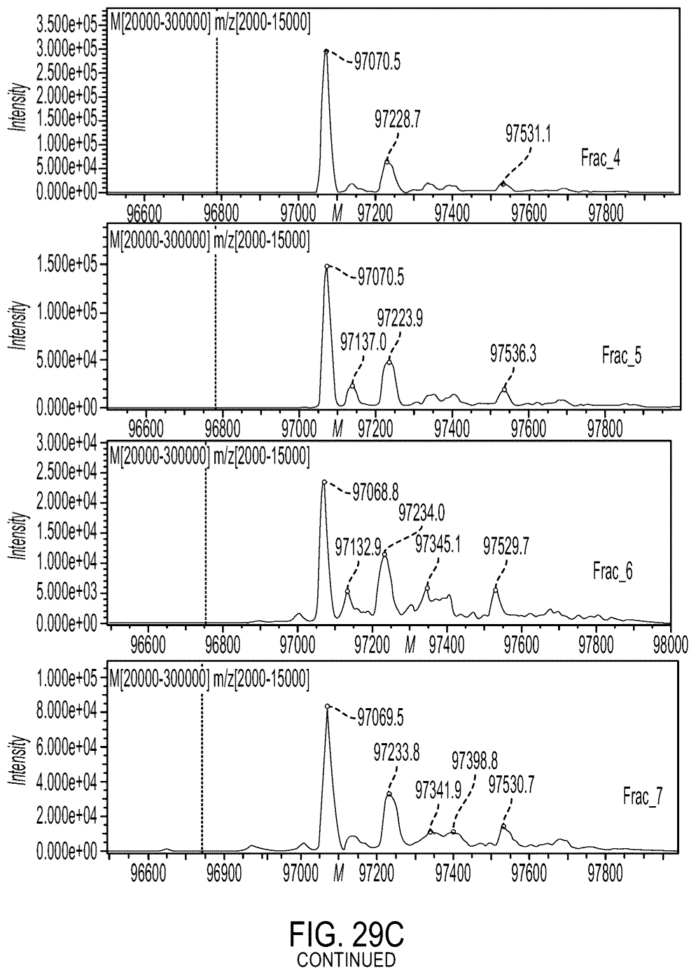

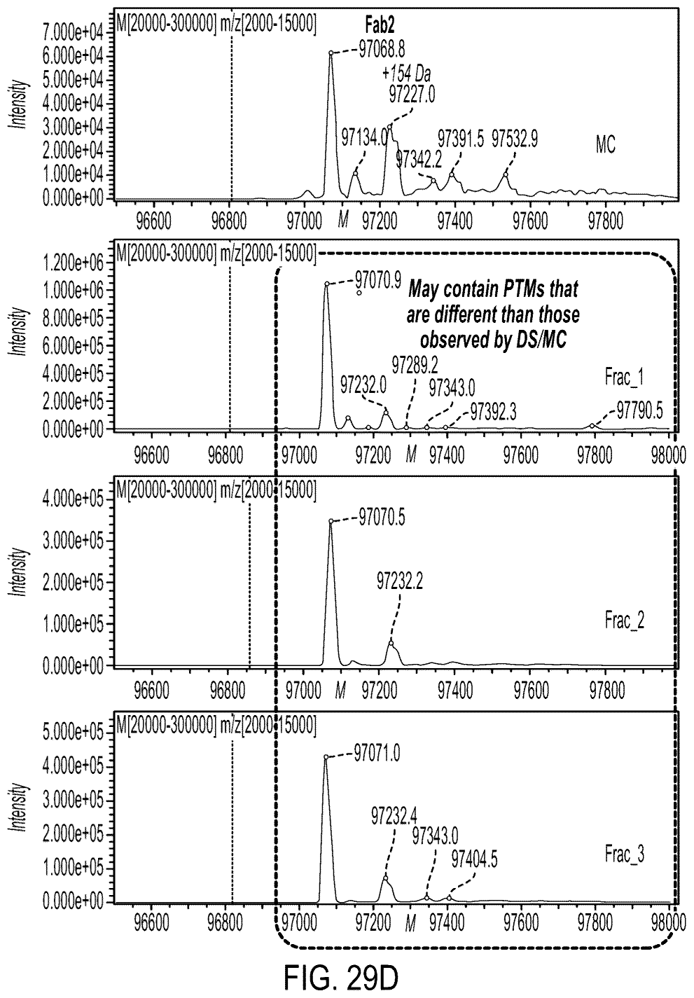

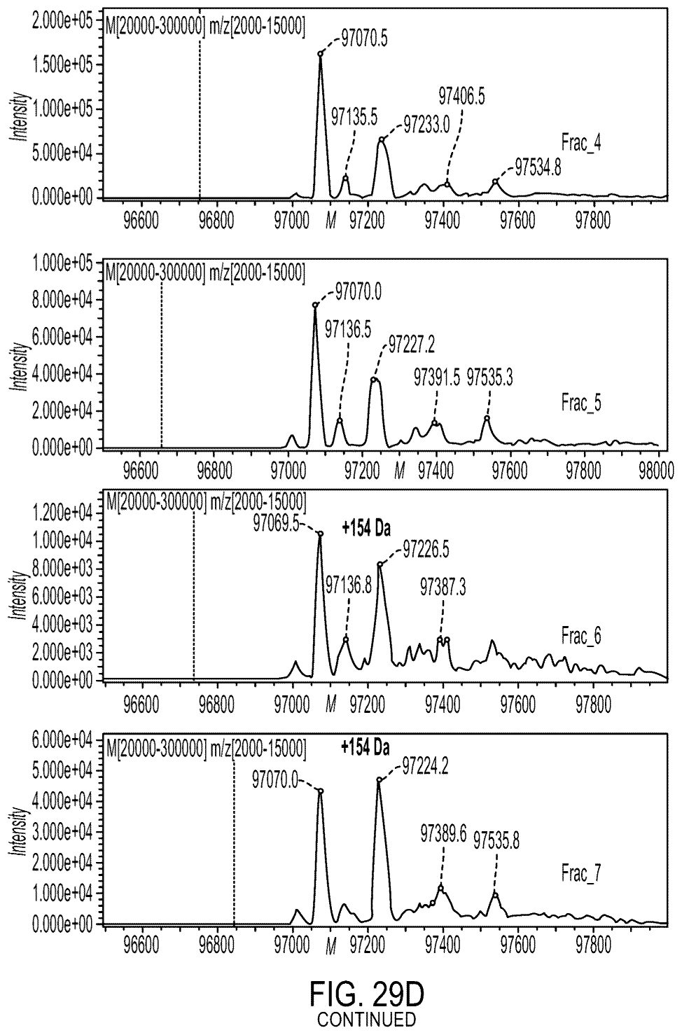

Mass spectra for each of the acidic variants from Fab fragments from each of the affinity enrichment fractions were compared, as shown in FIG. 29A, FIG. 29B, FIG. 29C, and FIG. 29D for peaks A4, A3, A2, and A1 respectively. In particular, peaks A2 and A1 from affinity enrichment fractions 1 to 3 showed potential variants that may not be observed in drug substance.

In conclusion, the methods of the present disclosure were useful for in-depth identification and characterization of variants of a protein of interest. Fractionation of a protein of interest based on binding affinity using affinity enrichment produced corresponded to binding affinity measured using a ligand binding assay, as shown in FIGS. 14A-14B, and measured using surface plasmon resonance, as shown in FIGS. 17A-17B. Fractions with reduced binding affinity (fractions 1-3) also corresponded to protein variants with reduced drug potency, as shown in FIG. 16. SE-UPLC analysis was useful for determining that reduced potency in msAb-3 is most correlated with LMW species lacking one or both Fab arms, as shown in FIG. 18. Native iCIEF analysis was useful for determining that reduced potency in msAb-3 is also correlated with an increase in acidic variants, as shown in FIG. 21. The specific identities of variant species present in each acidic and basic variant for each affinity fraction could be interrogated using native SCX-UV-MS, as shown in FIGS. 25A-25G. Adding subunit analysis provided additional information on the variant species in a sample of a protein of interest, as shown in FIGS. 27A-27D.

What is claimed is:

1. A method for identifying at least one critical quality attribute (CQA) of a protein of interest, comprising:
(a) contacting a sample including a protein of interest to a first target molecule, wherein said protein of interest binds to said first target molecule and said first target molecule is immobilized to a solid surface;
(b) eluting said protein of interest from said solid surface to collect at least two fractions;
(c) subjecting a first portion of said at least two fractions to surface plasmon resonance (SPR) analysis to characterize the binding of said protein of interest to a second target molecule;
(d) subjecting a second portion of said at least two fractions to mass spectrometry (MS) analysis to identify variants of said protein of interest; and
(e) comparing said variants to identify at least one CQA of said protein of interest.

2. The method of claim 1, wherein said protein of interest is selected from a group consisting of an antibody, a monospecific antibody, a bispecific antibody, a trispecific antibody, an antibody-derived protein, an antigen-binding protein, a fusion protein, an Fc-fusion protein, a receptor, a receptor ligand, a therapeutic protein, a fragment thereof, and a combination thereof.

3. The method of claim 1, wherein said first target molecule and/or said second target molecule is selected from a group consisting of an antibody, an antigen, a receptor, a receptor ligand, a therapeutic target, a fragment thereof, and a combination thereof.

4. The method of claim 1, wherein said solid surface is selected from a group consisting of a microplate, resin, beads, agarose beads, and magnetic beads.

5. The method of claim 1, further comprising immobilizing said first target molecule to said solid surface prior to step (a).

6. The method of claim 5, wherein said immobilizing comprises contacting a biotinylated first target molecule to a solid surface that is coated with avidin, streptavidin, or a variant thereof.

7. The method of claim 1, wherein said eluting comprises contacting said solid surface to an elution buffer.

8. The method of claim 7, wherein a pH of said elution buffer is increased or decreased over time.

9. The method of claim 1, wherein a number of said fractions is from 2 to 20, about 4, about 5, about 6, about 7, about 8, about 9, or about 10.

10. The method of claim 1, wherein said fractions comprise variants of said protein of interest with modified binding affinity to said first target molecule.

11. The method of claim 1, wherein said first target molecule is the same as said second target molecule.

12. The method of claim 1, wherein the determination of a CQA is based on the binding of said protein of interest to said first target molecule and/or said second target molecule.

13. The method of claim 1, further comprising subjecting said at least two fractions to a digestion step prior to step (c).

14. The method of claim 13, wherein said digestion step comprises contacting each of said at least two fractions to at least one digestive enzyme.

15. The method of claim 14, wherein said at least one digestive enzyme is selected from a group consisting of pepsin, trypsin, Tryp-N, chymotrypsin, Lys-N, Lys-C, Asp-N, Arg-C, Glu-C, papain, IdeS, variants thereof, and combinations thereof.

16. The method of claim 1, further comprising subjecting said at least two fractions to a separation step prior to MS analysis.

17. The method of claim 16, wherein said separation step comprises chromatography or electrophoresis.

18. The method of claim 17, wherein said chromatography comprises reverse phase chromatography, normal phase chromatography, hydrophobic interaction chromatography, hydrophilic interaction chromatography, ion exchange chromatography, anion exchange chromatography, cation exchange chromatography, strong cation exchange chromatography, weak cation exchange chromatography, size exclusion chromatography, mixed-mode chromatography, or a combination thereof.

19. The method of claim 17, wherein said electrophoresis comprises capillary electrophoresis, isoelectric focusing, or imaged capillary isoelectric focusing.

20. The method of claim 1, wherein said variants comprise acidic variants, basic variants, aggregates, crosslinking products, degradation products, truncation products, acylation, amidation, glycosylation, deglycosylation, oxidation, C-terminal lysine variation, N-terminal pyroglutamate variation, succinimide formation, iodination, acetylation, alkylation, methylation, adenylation, ADP-ribosylation, sulfonation, prenylation, hydroxylation, amidation, glutamylation, glycylation, isoprenylation, lipoylation, phosphopantetheinylation, phosphorylation, sulfation, citrullination, deamidation, SUMOylation, ubiquitination, glycation, glucuronylation, sialylation, or combinations thereof.

21. The method of claim 1, wherein said at least one CQA comprises acidic variants, basic variants, aggregates, crosslinking products, degradation products, truncation products, acylation, amidation, glycosylation, deglycosylation, oxidation, C-terminal lysine variation, N-terminal pyroglutamate variation, succinimide formation, iodination, acetylation, alkylation, methylation, adenylation, ADP-ribosylation, sulfonation, prenylation, hydroxylation, amidation, glutamylation, glycylation, isoprenylation, lipoylation, phosphopantetheinylation, phosphorylation, sulfation, citrullination, deamidation, SUMOylation, ubiquitination, glycation, glucuronylation, sialylation, or combinations thereof.

22. The method of claim 1, wherein a critical quality attribute is identified based on being an attribute of said protein of interest that is in higher abundance in a fraction that elutes earlier than another fraction.

23. The method of claim 1, wherein said at least one CQA is a post-translational modification.

24. The method of claim 1, wherein said protein of interest is an antigen-binding protein and said at least one CQA is in a complementarity-determining region of said antigen-binding protein.

25. The method of claim 1, wherein an amount of said first target molecule is insufficient to bind all of said protein of interest.

26. A method for characterizing binding variants of a protein of interest, comprising:
(a) contacting a sample including a protein of interest to a first target molecule, wherein said protein of interest binds to said first target molecule and said first target molecule is immobilized to a solid surface;
(b) eluting said protein of interest from said solid surface to collect at least two fractions;
(c) subjecting a first portion of said at least two fractions to surface plasmon resonance (SPR) analysis to characterize the binding of said protein of interest to a second target molecule;
(d) subjecting a second portion of each of said at least two fractions to separation by size or charge to produce a separation profile; and
(e) comparing said binding and said separation profiles to characterize binding variants of said protein of interest.

27. The method of claim 26, wherein said protein of interest is selected from a group consisting of an antibody, a monospecific antibody, a bispecific antibody, a trispecific antibody, an antibody-derived protein, an antigen-binding protein, a fusion protein, an Fe-fusion protein, a receptor, a receptor ligand, a therapeutic protein, a fragment thereof, and a combination thereof.

28. The method of claim 26, wherein said first target molecule and/or said second target molecule is selected from a group consisting of an antibody, an antigen, a receptor, a receptor ligand, a therapeutic target, a fragment thereof, and a combination thereof.

29. The method of claim 26, wherein said solid surface is selected from a group consisting of a microplate, resin, beads, agarose beads, and magnetic beads.

30. The method of claim 26, further comprising immobilizing said first target molecule to said solid surface prior to step (a).

31. The method of claim 30, wherein said immobilizing comprises contacting a biotinylated first target molecule to a solid surface that is coated with avidin, streptavidin, or a variant thereof.

32. The method of claim 26, wherein said eluting comprises contacting said solid surface to an elution buffer.

33. The method of claim 32, wherein a pH of said elution buffer is increased or decreased over time.

34. The method of claim 26, wherein a number of said fractions is from 2 to 20, about 4, about 5, about 6, about 7, about 8, about 9, or about 10.

35. The method of claim 26, wherein said fractions comprise variants of said protein of interest with modified binding affinity to said first target molecule.

36. The method of claim 26, wherein said first target molecule is the same as said second target molecule.

37. The method of claim 26, further comprising subjecting said at least two fractions to a digestion step prior to step (c).

38. The method of claim 37, wherein said digestion step comprises contacting each of said at least two fractions to at least one digestive enzyme.

39. The method of claim 38, wherein said at least one digestive enzyme is selected from a group consisting of pepsin, trypsin, Tryp-N, chymotrypsin, Lys-N, Lys-C, Asp-N, Arg-C, Glu-C, papain, IdeS, variants thereof, and combinations thereof.

40. The method of claim 26, wherein said separation comprises chromatography or electrophoresis.

41. The method of claim 40, wherein said chromatography comprises reverse phase chromatography, normal phase chromatography, hydrophobic interaction chromatography, hydrophilic interaction chromatography, ion exchange chromatography, anion exchange chromatography, cation exchange chromatography, strong cation exchange chromatography, weak cation exchange chromatography, size exclusion chromatography, mixed-mode chromatography, or a combination thereof.

42. The method of claim 40, wherein said electrophoresis comprises capillary electrophoresis, isoelectric focusing, or imaged capillary isoelectric focusing.

43. The method of claim 26, wherein producing said separation profile comprises measuring said protein of interest using ultraviolet detection or fluorescence detection.

44. The method of claim 26, wherein said binding variants comprise acidic variants, basic variants, aggregates, crosslinking products, degradation products, truncation products, acylation, amidation, glycosylation, deglycosylation, oxidation, C-terminal lysine variation, N-terminal pyroglutamate variation, succinimide formation, iodination, acetylation, alkylation, methylation, adenylation, ADP-ribosylation, sulfonation, prenylation, hydroxylation, amidation, glutamylation, glycylation, isoprenylation, lipoylation, phosphopantetheinylation, phosphorylation, sulfation, citrullination, deamidation, SUMOylation, ubiquitination, glycation, glucuronylation, sialylation, or combinations thereof.

45. The method of claim 26, further comprising subjecting said separated fractions to MS analysis to quantify, characterize, and/or identify said binding variants.

46. The method of claim 26, wherein said binding variant has reduced binding compared to a main species of said protein of interest.

47. The method of claim 26, wherein an amount of said first target molecule is insufficient to bind all of said protein of interest.
